(19) United States
(12) Patent Application Publication
(10) Pub. No.: US 2023/0390708 A1
(43) Pub. Date: Dec. 7, 2023

(54) CHARGED MEMBRANES INCORPORATED WITH POROUS POLYMER FRAMEWORKS

(71) Applicant: The Regents of the University of California, Oakland, CA (US)

(72) Inventors: Jeffrey R. Long, Lafayette, CA (US); Adam Andrew Uliana, Bethlehem, PA (US); Jeffrey J. Urban, Emeryville, CA (US); Ngoc Bui, Berkely, CA (US)

(21) Appl. No.: 18/026,480

(22) PCT Filed: Sep. 16, 2021
(86) PCT No.: PCT/US2021/050738

§ 371 Date: Mar. 15, 2023

Related U.S. Application Data
(60) Provisional application No. 63/079,457, filed on Sep. 16, 2020.
(60) Provisional application No. 63/118,322, filed on Nov. 25, 2020.

(51) Int. Cl. B01D 69/12; B01D 69/02; B01D 71/68; B01D 71/82; C02F 1/42

(52) U.S. Cl. CPC B01D 69/12; B01D 69/02; B01D 71/68; B01D 71/82; C02F 1/42; C02F 2001/425; B01D 2257/60; B01D 2257/504; B01D 2257/304; B01D 2257/102; B01D 2257/80; B01D 2257/302

(57) ABSTRACT

The disclosure relates to composite membranes for selective separation and processing of fluids and industrial applications.

### CROSS REFERENCE TO RELATED APPLICATIONS

This application claims priority under 35 U.S.C. § 119 from U.S. Provisional Application Ser. No. 63/079,457, filed Sep. 16, 2020, and U.S. Provisional Application Ser. No. 63/118,322, filed Nov. 25, 2020, the disclosures of each of which are incorporated herein by reference in their entirety.

### STATEMENT REGARDING FEDERALLY SPONOSORED RESEARCH

This invention was made with Government support under DE-SC0001015 awarded by the U.S. Department of Energy, and under LB18010 awarded by the U.S. Department of Energy. The government has certain rights in the invention.

### TECHNICAL FIELD

The disclosure relates to membranes and membranes systems for the separation of trace components in a fluid mixture. The disclosure provides for composite membranes that are comprised of a polymer/membrane matrix which contains or is embedded with porous aromatic frameworks, and uses thereof.

### BACKGROUND

Up to 10-15% of the global energy consumption is used on chemical separations, and traditional heat-driven separations such as distillation account for roughly 80% of this separation related energy. While membrane-based separations are up to 10 times more energy-efficient than heat-driven processes, membrane technologies are still underdeveloped or expensive. In particular, advanced membranes that can selectively isolate trace components of interest from various mixtures must be developed, as these difficult separations make up several prime “holy grail” targets in the separations industry within the coming century. For example, micropollutants such as heavy metal ions are often found in various water sources at trace yet toxic concentrations alongside relatively nontoxic components (e.g., sodium ions) that are several orders of magnitude more concentrated. Similarly, 1,000 times more uranium exists naturally in seawater than in geological reserves, but commercial materials cannot effectively isolate uranium from this complex aqueous solution. The capture of other minor components from complex gas mixtures, such as carbon dioxide from air or exhaust streams, is also urgently needed for environmental preservation.

Over the past several decades, ion exchange, adsorption, and membrane processes have each been widely studied and applied for the separation of various liquid and gas mixtures. However, commercial materials and methods seldom possess the exceptional selectivity and throughput required to isolate minor components of interest from these mixtures, necessitating additional energy-intensive stages and processes to achieve desired targets. Ion exchange resins, for example, rely on electrostatic attractive forces to remove trace toxic ions. Nonetheless, these commercial materials do not possess the precisely controlled pore sizes and chemical functionalities needed to selectively capture trace target ions from solutions containing abundant competing ions with similar charge. Likewise, the low and uncontrolled porosities of commercial adsorbents lead to low functional group loadings and slow mass transfer kinetics. For the case of water purification, electrodialysis and reverse osmosis are currently among the most commonly used membrane-based desalination technologies. However, similar to other membrane technologies, these approaches aim to separate water from all ions and thus return toxic ions to the environment with the concentrated brine solution (˜50% of the feed volume for reverse osmosis); thus, these ions of interest cannot be captured for proper disposal or for re-use as a commodity material.8 Hence, the development of novel, highly selective materials and methods is urgently needed to recover minor components of interest from various liquid and gas mixtures.

### SUMMARY

The selective separation of trace components of interest from various mixtures (e.g., micropollutants from groundwater, lithium or uranium from seawater, carbon dioxide from air) presents an especially pressing technological challenge. Established materials and separation processes seldom meet the performance standards needed to efficiently isolate these trace species for proper disposal or re-use. To address this issue, the disclosure provides for the development of a novel separation strategy in which highly selective and tunable MOFs, COFs, ZIFs and/or PAFs are embedded into membranes. In this approach, the minor target species are selectively captured by the embedded MOFs, COFs, ZIFs and/or PAFs while the species transport through the membrane. Simultaneously, the mixture can be purified through traditional membrane separation mechanisms. As shown in the studies presented herein, composite membranes which have incorporated Hg²⁺-selective PAFs into electrodialysis membranes that can simultaneously capture He via an adsorption mechanism while desalinating water through an electrodialysis mechanism. Adsorption studies demonstrate that the embedded PAFs maintained rapid, selective, regenerable, and high-capacity Hg²⁺ binding capabilities within the membrane matrix. Furthermore, when inserted into an electrodialysis setup, the composite membranes successfully capture all Hg²⁺ from various Hg²⁺-spiked water sources while permeating all other competing cations to simultaneously enable desalination. Finally, using an array of other ion-selective adsorbents, the composite membranes, in principle, may be applied generally to any target ion present in any water source. The composite membranes can be applied to existing membrane processes to efficiently capture targeted species of interest, without the need for additional expensive equipment or processes such as fixed-bed adsorption columns.

Ion-exchange membranes have been established for a variety of industrial applications, including energy and environmental technologies related to water treatment, fuel cells, and flow batteries. The limited tunability and adverse ion permeability-selectivity tradeoff exhibited by traditional ion-exchange membranes, however, limit their development. The disclosure provides for a new class of composite ion-exchange membrane materials incorporated with highly tunable porous aromatic frameworks (PAFs). In the studies presented herein, an assortment of PAF variants can be easily embedded into charged membranes, where the choice of PAF filler can be used to optimize the physical, ion transport, and adsorptive properties of the membrane according to their targeted application. Material characterizations indicate that numerous charged membranes embedded with PAFs exhibit excellent dispersibility, interfacial compatibility, structural flexibility, and pH stability. Proton conductivity and water uptake measurements also indicate that the exceptionally high porosity of PAFs enhances ion diffusion in membranes, while abundant, favorable PAF-polymer interactions decrease non-selective swelling pathways typically observed in highly charged ion-exchange membranes. Furthermore, adsorption experiments demonstrate that ion-selective PAFs can be embedded into charged membranes to tune the ion selectivity of the membrane and also enable their use as membrane adsorbents. Accordingly, the disclosure provides for notable improvement in the general performance and tunability of ion-exchange membrane technologies.

In a particular embodiment, the disclosure provides for a composite membrane comprising a polymer/membrane matrix that contains or is embedded with one or more metal organic frameworks (MOFs), covalent organic frameworks (COFs), zeolitic imidazolate frameworks (ZIFs), and/or porous aromatic frameworks (PAFs) that selectively binds to one or more targeted ions or organic molecules. In another embodiment, the polymer/membrane matrix comprises ion exchange polymer/membrane matrix materials. In yet another embodiment, the ion exchange polymer/membrane matrix materials is made from dimethyl-2-hydroxy benzyl amine, phenol and formaldehyde; C₆H₄ (OH)₂ or 1,2,3-C₆H₃ (OH)₃, NH₂C₆H₄COOH, and formaldehyde; benzidine-formaldehyde and acrylonitrile-vinyl chloride copolymer; phenolsulfonic acid and formaldehyde; m-phenylene diamine or aliphatic diamine compounds and formaldehyde; tetrafluoroethylene and vinyl-ether; sulfonation and amination of styrene and divinylbenzene polymers; and sulfonated polysulfone. In a further embodiment, the ion exchange polymer/membrane matrix material is sulfonated polysulfone. In a certain embodiment, the composite membrane contains the one or more MOFs, COFS, ZIFs, and/or PAFs at a weight percent of 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, or a range that includes or is between any two of the foregoing values (e.g., 10 wt % to 25 wt %). In a further embodiment, the composite membrane comprises one or more PAFs. In yet a further embodiment, the one or more PAFs are selected from PAF-1, PAF-1-CH₃, PAF-1-CH₂OH, PAF-1-CH₂-phthalimide, PAF-1-CH₂N═CMe₂, PAF-1-CH₂Cl, PAF-1-SH, PAF-1-ET, PAF-1-NMDG, PAF-1-SMe, PAF-1-CH₂NH₂, and PAF-1-CH₂AO (wherein AO is an amidoxime group). In a certain embodiment, the one or more PAFs are selected from PAF-1-SH, PAF-1-ET, PAF-1-NMDG, PAF-1-SMe, PAF-1-CH₂NH₂, and PAF-1-CH₂AO. In another embodiment, the targeted ions are selected from Hg²⁺, Nd³⁺, Cu²⁺, Pb²⁺, UO₂²⁺, B(OH)₃, Fe³⁺, and AuCl₄⁻.

In a particular embodiment, the disclosure also provides a method for ion-capture electrodialysis (IC-ED) comprising: contacting a fluid feedstock with a composite membrane of the disclosure, wherein the feedstock comprises targeted ions; applying an external voltage to the feedstock to generate an electric potential gradient; capturing targeted ions or organic molecules in the composite membrane; recovering the fluid that passes through the composite membrane; wherein the recovered fluid comprises significantly less or nondetectable levels of the targeted ions in comparison to the feedstock. In another embodiment, the targeted ions are selected from Hg²⁺, Nd³⁺, Cu²⁺, Pb²⁺, UO₂²⁺, B(OH)₃, Fe³⁺, and AuCl₄⁻. In yet another embodiment, the feedstock is selected from seawater, industrial wastewater, groundwater, and brackish water. In a further embodiment, the recovered fluid is desalinated water. In yet a further embodiment, >99% of the targeted ions are captured by the composite membrane. In another embodiment, the method provides for selective capture of B(OH)₃ from seawater or brackish water. In yet another embodiment, the method provides for selective capture of Hg²⁺, Nd³⁺, Pb²⁺, Fe³⁺ and/or UO₂²⁺ from industrial wastewater.

The disclosure provides a composite membrane comprising a polymer/membrane matrix that contains or is embedded with particles comprising one or more types of porous aromatic frameworks (PAFs), wherein the one or more types of PAFs comprise functional groups that bind with a high specificity to a targeted ion, organic molecule, or contaminant. In one embodiment, the polymer/membrane matrix comprises an ion exchange polymer/membrane matrix material. In a further embodiment, the ion exchange polymer/membrane matrix material is a cation exchange polymer/membrane matrix material. In yet another or further embodiment, the ion exchange polymer/membrane matrix material is a anion exchange polymer/membrane matrix material. In still another or further embodiment, the ion exchange polymer/membrane matrix materials is made from dimethyl-2-hydroxy benzyl amine, phenol and formaldehyde; C₆H₄ (OH)₂ or 1,2,3-C₆H₃(OH)₃, NH₂C₆H₄COOH, and formaldehyde; benzidine-formaldehyde and acrylonitrile-vinyl chloride copolymer; phenolsulfonic acid and formaldehyde; m-phenylene diamine or aliphatic diamine compounds and formaldehyde; tetrafluoroethylene and vinyl-ether; sulfonation and amination of styrene and divinylbenzene polymers; and sulfonated polysulfone. In a further embodiment, the ion exchange polymer/membrane matrix material is made from sulfonated polysulfone. In another embodiment, the particles are from 50 nm to 300 nm in diameter. In another embodiment, the particles are evenly dispersed in the polymer/membrane matrix material. In yet another embodiment, the composite membrane contains from 5 wt % to 40 wt % of the one or more types of PAFs. In a further embodiment, the composite membrane contains from 10 wt % to 25 wt % of the one or more types of PAFs. In another embodiment, the one or more types of PAFs comprise a series of nodes linked together by linking ligands, wherein the series of nodes have a formula selected from Formula I or Formula II:

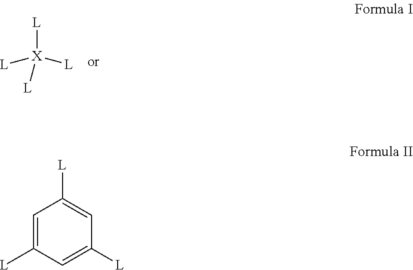

wherein, X is selected from C, B⁻ and P⁺; and L is a linking ligand;
and wherein the linking ligand has a structure of Formula III:

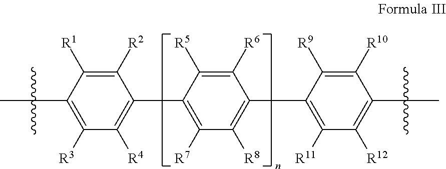

wherein, R¹-R¹² are independently selected from H, an optionally substituted (C₁-C₆)alkyl, an optionally substituted (C₁-C₆)alkenyl, an optionally substituted (C1-C5)-O-(C₁-C₆)alkyl, halo, —OH, —CH₂R¹³, —CO₂H, —CO₂R¹⁴, —SH, —SMe, —SO₂H, —SO₃H, —NR¹⁵R¹⁶, —N⁺(H)₃, —N⁺(CH₃)₃, cyano, amide, azide, —PO₃H, —B(OR¹⁴)₂, 2-(methylthio)ethan-1-ol, N-methyl-D-glucamine, and heterocycle; R¹³ is selected from H, —OH, halo, —NH₂, —NR¹⁵R¹⁶, —N═C(CH₃)₂, -phthalimide, —C(NH₂)═N—OH, —SH, —SMe, —SO₂H, —SO₃H, —N⁺(H)₃, —N⁺(CH₃)₃, —PO₃H, —O-(C₁-C₆) alkyl, cyano, amide, azide, —B(OR¹⁴)₂, - and heterocycle; R¹⁴ to R¹⁶ are independently selected from H or an optionally substituted (C₁-C₆)alkyl; and n is an integer selected from 0, 1, 2, 3, 4, or 5. In still another embodiment, the one or more types of PAFs are selected from PAF-1, PAF-1-CH₃, PAF-1-CH₂OH, PAF-1-CH₂-phthalimide, PAF-1-CH₂N═CMe₂, PAF-1-CH₂Cl, PAF-1-SH, PAF-1-ET (wherein ET is 2-(methylthio)ethan-1-ol), PAF-1-NMDG (wherein NMDG is N-methyl-D-glucamine), PAF-1-SMe, PAF-1-CH₂NH₂, and PAF-1-CH₂AO (wherein AO is an amidoxime group). In a further embodiment, the one or more types of PAFs are selected from PAF-1-SH, PAF-1-ET, PAF-1-NMDG, PAF-1-SMe, PAF-1-CH₂NH₂, and PAF-1-CH₂AO. In another embodiment, the one or more types of PAFs bind with a high specificity to a targeted ion selected from Hg²⁺, Nd³⁺, Cu²⁺, Pb²⁺, UO₂²⁺, B(OH)₃, Fe³⁺, and AuCl₄⁻.

The disclosure also provide a method for removing a targeted ion, organic molecule, or contaminant from a fluid feedstock comprising contacting a fluid feedstock with a composite membrane comprising a polymer/membrane matrix that contains or is embedded with particles comprising one or more types of porous aromatic frameworks (PAFs), wherein the one or more types of PAFs comprise functional groups that bind with a high specificity to a targeted ion, organic molecule, or contaminant, wherein the fluid feedstock comprises the targeted ion, organic molecule, or contaminant, wherein the targeted ion, organic molecule, or contaminant is absorbed or captured by the PAFs present in the composite membrane. In one embodiment, the fluid feedstock is used in an application or process selected from: electrodialysis, membrane capacitive deionization, electrofiltration, fuel cells, fuel gas streams, gas purification, and CO₂ capture. In another embodiment, the non-targeted ions, organic molecules, or contaminants flow through the membrane and are not absorbed or captured by the PAFs present in the composite membrane. In still another embodiment, the fluid feedstock comprises a targeted ion selected from Hg²⁺, Nd³⁺, Cu²⁺, Pb²⁺, UO₂²⁺, B(OH)₃, Fe³⁺, and AuCl₄⁻. In yet another embodiment, the fluid feedstock comprises a targeted contaminant selected from carbon dioxide, hydrogen sulfide, nitrogen, water, and sulfur oxide. In yet another embodiment of any of the foregoing embodiments, the composite membrane comp: s a porous aromatic framework comprising a series of nodes linked together by linking ligands, wherein the series of nodes have a formula selected from Formula I or Formula II:

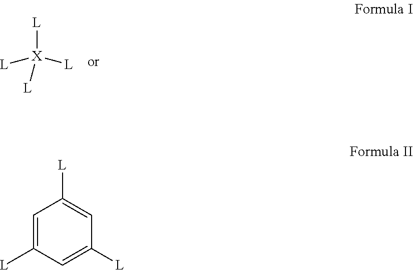

wherein, X is selected from C, B⁻ and P⁺; and L is a linking ligand;
and wherein the linking ligand has a structure of Formula III:

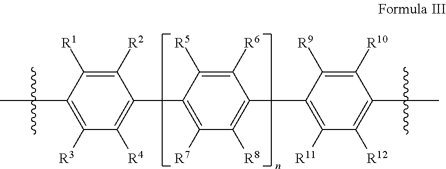

wherein, R¹-R¹² are independently selected from H, an optionally substituted (C₁-C₆)alkyl, an optionally substituted (C₁-C₆)alkenyl, an optionally substituted (C1-C5)-O-(C₁-C₆)alkyl, halo, —OH, —CH₂R¹³, —CO₂H, —COR¹⁴, —CO₂R¹⁴, —SH, —SMe, —SO₂H, —SO₃H, —NR¹⁵R¹⁶, —N⁺(H)₃, —N⁺(CH₃)₃, cyano, amide, azide, —PO₃H, —B(OR¹⁴)₂, 2-(methylthio)ethan-1-ol, N-methyl-D-glucamine, and heterocycle; R¹³ is selected from H, —OH, halo, —NH₂, —NR¹⁵R¹⁶, —N═C(CH₃)₂, -phthalimide, —C(NH₂)═N—OH, —SH, —SMe, —SO₂H, —SO₃H, —N⁺(H)₃, —N⁺(CH₃)₃, —PO₃H, cyano, amide, azide, —B(OR¹⁴)₂, - and heterocycle; R¹⁴ to R¹⁶ are independently selected from H or an optionally substituted (C₁-C₆)alkyl; and n is an integer selected from 0, 1, 2, 3, 4, or 5.

### DESCRIPTION OF DRAWINGS

FIG. 1A-D shows a design of composite membranes and application in ion-capture electrodialysis (IC-ED). (A and B) Tunable composite membranes were prepared by embedding PAFs with selective ion binding sites into cation exchange polymer matrices. (C) Demonstrates the use of these adsorptive membranes in an electrodialysis-based process for the selective capture of target cations (right-hand side) from water and simultaneous desalination. Water splitting occurs at both electrodes to maintain electroneutrality. (D) Cross-sectional scanning electron micrographs (expanded view in inset) revealed high PAF dispersibility and strong, favorable interactions between the PAF and polymer matrix.

FIG. 2A-E shows Properties of PAF-embedded ion exchange membranes. (A,B) Composite membranes exhibit increasing water uptake, swelling resistance, and glass transition temperature (T_{g}) with increasing PAF-1-SH loading. (C) Comparison of equilibrium Hg²⁺ uptake in neat sPSF and sPSF with 20 wt % PAF-1-SH. Solid lines represent fits with a Langmuir model. Mercury ion uptake in the composite membrane closely approaches the predicted saturation uptake (329 mg/g) assuming all binding sites in the PAF particles are accessible. (D) Equilibrium uptake of Hg²⁺ in neat sPSF and sPSF with 20 wt % PAF-1-SH exposed to deionized (DI) water and various synthetic water samples with 100 ppm added Hg²⁺. (E) Mercury ion uptake in 20 wt % PAF-1-SH membranes as a function of cycle number. Minimal decrease in Hg²⁺ uptake occurs over 10 cycles. The initial He concentration was 100 ppm for each cycle, and all Hg²⁺ captured in each cycle was recovered using HCl and NaNO₃. Error bars denote ±1 standard deviation around the mean from at least three separate measurements.

FIG. 3A-D shows IC-ED of diverse water sources. Results from IC-ED of synthetic (A) groundwater, (B) brackish water, and (C) industrial wastewater containing 5 ppm Hg²⁺ using 20 wt % PAF-1-SH in sPSF (applied voltage: −4 V versus Ag/AgCl). All Hg²⁺ was selectively captured from the feeds (open circles) without detectable permeation into the receiving solutions (closed circles). (Insets) All other cations were transported across the membranes to desalinate the feeds. The long duration of the IC-ED tests is an artifact of the experimental setup rather than the materials or IC-ED method. (D) Breakthrough data for IC-ED using sPSF embedded with 10 or 20 wt % PAF-1-SH. Receiving Hg²⁺ concentrations are plotted against the amount of Hg²⁺ captured at different time intervals (in mg per gram of PAF-1-SH in each composite membrane). The predicted capacity (gray dotted line) corresponds to the Hg²⁺ uptake achieved using PAF-1-SH powder under analogous testing conditions. (Inset) Concentration of Hg²⁺ in the receiving solutions for IC-ED processes using neat sPSF (diamonds) and sPSF with 10 wt % PAF-1-SH (squares) and 20 wt % PAF-1-SH (circles), plotted versus time t normalized by the breakthrough time for the 20 wt % PAF-1-SH composite membrane, t₀. Mean values determined from two replicate experiments are shown. Initial feed: 100 ppm Hg²⁺ in 0.1 M NaNO₃; applied voltage: −2 V vs. Ag/AgCl.

FIG. 4A-C shows Tuning membranes to selectively recover various target solutes. (A) Cu⁺— and (B) Fe³⁺-capture electrodialysis (applied voltages: −2 and −1.5 V vs. Ag/AgCl, respectively) using composite membranes with 20 wt % PAF-1-SMe and PAF-1-ET in sPSF, respectively. HEPES buffer (0.1 M) was used as the source water in each solution to supply competing ions and maintain constant pH. The insets show the successful transport of all competing cations across the membrane to desalinate the feed. (C) B(OH)₃-capture diffusion dialysis of groundwater containing 4.5 ppm boron using composite membranes with 20 wt % PAF-1-NMDG in sPSF (no applied voltage). The inset shows results using neat sPSF membranes for comparison. Open and closed symbols denote feed and receiving concentrations, respectively. Each plot point represents the mean value determined from two replicate experiments. Gray dotted lines indicate recommended maximum contaminant limits imposed by the U.S. Environmental Protection Agency (EPA) for Cu²⁺, the EPA and World Health Organization for Fe³⁺, and agricultural restrictions for sensitive crops for B(OH)₃.

FIG. 5 shows a general scheme for the syntheses of sulfonated polysulfone (sPSF), the parent porous aromatic framework (PAF-1), and the post-synthetically functionalized PAF-1 variants. Reaction conditions: (i) polysulfone resin, chlorosulfonic acid, chloroform; (ii) Ni(cod)₂, cod, 2,2′-bipyridine, N,N-dimethylformamide, 80° C.; (iii) paraformaldehyde, acetic acid, H₃PO₄, HCl, 90° C.; (iv) sodium hydrosulfide, ethanol, reflux; (v)₂-(methylthio)ethanol, NaH, toluene, 90° C.; (vi) N-methyl-D-glucamine, N,N-dimethylformamide, 90° C.; (vii) sodium thiomethoxide, ethanol, 70° C.

FIG. 6 shows synthetic control of degree of sulfonation (sulfonate groups per PSF repeat unit) based on the molar ratio of chlorosulfonic acid to polysulfone (PSF) used. Degrees of sulfonation were calculated using ¹H NMR. Synthesized sPSF with degrees of sulfonation higher than 146% fall off of the linear trend, possibly as a result of sulfonation side reactions. Since functionalized sulfonate groups are electron withdrawing, further sulfonation is expected to be less favorable after high degrees of sulfonation have already been achieved, potentially enabling side reactions instead. Red diamonds represent sulfonated PSF materials that can form water-stable freestanding membranes upon casting, while light red squares represent sulfonated PSF materials that dissolve in water after membrane casting.

FIG. 7 shows 77 K nitrogen adsorption isotherms for PAF-1, PAF-1-SH, PAF-1-SMe, PAF-1-ET, and PAF-1-NMDG used to calculate BET surface areas. The expected drop in surface area upon the functionalization of PAF-1 likely results from the partial pore filling and added mass of the functional groups. Filled symbols denote adsorption, while open symbols denote desorption.

FIG. 8 shows a check of the first BET consistency criterion to identify the maximum P/P₀ value (indicated by dashed lines) that should be used for calculating the BET surface areas. The pressure range selected for BET surface area determination should possess values of n·(1−P/P₀) increasing with P/P₀ (69), where n denotes millimoles of N₂ adsorbed per gram of dry material.

FIG. 9 provides points used to determine the BET surface areas of PAF-1 and the functionalized PAF-1 variants. The y-intercept calculated from each trendline of best fit is a positive value, which fulfills the second BET consistency criterion (69). nₜₒₜₐₗ denotes moles of N₂ adsorbed in each sample at each point.

FIG. 10 shows 87 K argon adsorption isotherms for PAF-1, PAF-1-SH, PAF-1-SMe, PAF-1-ET, and PAF-1-NMDG used to calculate pore size distributions. Filled symbols denote adsorption, while open symbols denote desorption.

FIG. 11 shows pore size distributions of PAF-1 and its functionalized variants determined from Ar adsorption isotherms at 87 K.

FIG. 12 shows FTIR-ATR spectra of the synthesized PAFs.

FIG. 13 shows thermogravimetric analysis (TGA) decomposition profiles (5° C. min⁻¹ ramp rate with flowing N₂) of PAF-1, PAF-1-CH₂Cl, PAF-1-SH, PAF-1-SMe, PAF-1-ET, and PAF-1-NMDG powders.

FIG. 14A-B shows characterization of PAF-1-SH particle sizes. (A) Number-averaged particle size distributions of PAF-1-SH dispersed in the DMF casting solvent, as measured by dynamic light scattering. The median diameter (d₅₀) was 206 nm. Particle sizes measured around ˜600-1,000 nm are likely attributed to agglomerations of a few particles. (B) Field emission SEM image of a single PAF-1-SH particle, which features a diameter of ˜200 nm. The size and morphology of the particle closely resemble that of membrane-embedded PAFs observed in cross-sectional membrane SEM images (FIG. 1D). Scale bar: 50 nm.

FIG. 15A-B shows (A) Thermogravimetric analysis (TGA) decomposition profiles (5° C. min⁻¹ ramp rate with flowing N₂) of PAF-1-SH powder and fabricated membranes with different PAF-1-SH wt % loadings in sulfonated polysulfone (sPSF). (B) TGA profiles of composite membranes compared to expected profiles. Each expected profile was calculated as the corresponding weighted average of the obtained PAF-1-SH and neat sPSF TGA profiles.

FIG. 16 shows Membrane dissolution studies to investigate the abundance and strength of favorable interfacial interactions between PAFs and the polymer matrix. While neat sulfonated polysulfone (sPSF) membranes are partially or completely soluble in various casting solvents as expected, composite films containing PAFs exhibit increased stability and become completely or partially insoluble in these solvents as a result of strong PAF/polymer interfacial interactions. Leaching of PAF particles from composite membranes is also not observed upon immersion in water, concentrated acid, or concentrated base.

FIG. 17 shows static DI water contact angles of membranes consisting of neat polysulfone (PSF), neat sulfonated polysulfone (sPSF), or different loadings (5, 10, 15, or 20 wt %) of PAF-1-SH in sPSF. No significant differences in contact angle were observed in sPSF membranes with different PAF loadings. This uniformity suggests that the PAFs do not significantly contribute to surface hydrophilicity or roughness and are likely embedded inside of the membrane matrix rather than on the surface. Reported values and error bars represent the mean and standard deviation, respectively, obtained from measurements on five randomly selected locations on each sample.

FIG. 18 provides a plot of Hg²⁺ equilibrium adsorption isotherm for PAF-1-SH. Approximately 100% of the thiol binding groups in PAF-1-SH (thiol loading calculated from sulfur elemental analysis) are utilized for Hg²⁺ capture at saturation with a 1:1 binding ratio of thiol to Hg²⁺. A single-site Langmuir model was used to fit the data.

FIG. 19 shows batch equilibrium adsorption of Hg(NO₃)₂ and HgCl₂ by PAF-1-SH powder. Small differences in Hg²⁺ uptake (˜30 mg g⁻¹) are obtained when different counterions are present in solution. The initial Hg²⁺ concentration in the testing solutions was ˜100 ppm. Reported values and error bars represent the mean and standard deviation, respectively, obtained from measurements on at least three different samples.

FIG. 20 provides plots of Hg²⁺ equilibrium adsorption data for PAF-1-SH powder and neat sulfonated polysulfone (sPSF) membranes, fitted with the linearized single-site Langmuir model. Trendlines were fit using linear regression.

FIG. 21 is a plot showing Hg²⁺ adsorption kinetics for PAF-1-SH powder. The initial Hg²⁺ concentration in the testing solution was 100 ppm. The first data point was taken 10 s after the He solution was added. By 10 s, 81% of the Hg²⁺ equilibrium capacity was already reached. Rapid binding kinetics by PAF-1-SH are likely attributed to the high porosities and small particle sizes of PAF-1-SH, which minimize mass transfer resistances.

FIG. 22 is a plot showing Hg²⁺ adsorption kinetics for a neat sulfonated polysulfone (sPSF) membrane (red diamonds) and a 20 wt % PAF-1-SH in sPSF membrane (blue circles), including an expanded view (inset) of the first ˜2 h of adsorption. The initial Hg²⁺ concentration in each testing solution was 150 ppm. After 1 h, both membranes achieved ˜80% of their Hg²⁺ equilibrium capacities. These drastically slower Hg²⁺ adsorption kinetics, compared to that of bulk PAF-1-SH (FIG. 21), suggest that Hg²⁺ adsorption in membrane-embedded PAF-1-SH is limited by diffusion through the sPSF matrix.

FIG. 23 shows (Top) Single-component equilibrium uptake of Hg²⁺ and various common waterborne ions by PAF-1-SH powder (initial concentrations: 0.5 mM). (Bottom) Equilibrium adsorption of Hg²⁺ by PAF-1-SH powder in different realistic water solutions with 100 ppm added Hg²⁺. Uptake of Hg²⁺ by PAF-1-SH from a solution of only Hg²⁺ only (100 ppm) in DI water is also shown for comparison. No loss in Hg²⁺ capacity occurs in the presence of various abundant competing ions in each solution, indicating exceptional multicomponent selectivity of PAF-1-SH for Hg²⁺. Reported values and error bars in each figure represent the mean and standard deviation, respectively, obtained from measurements on at least three different samples.

FIG. 24 shows a plot obtained from electrodialysis of synthetic groundwater containing ˜5 ppm Hg²⁺ using a neat sPSF membrane; 7.5-mL half-cells were used, and −4 V vs. Ag/AgCl were applied across the cell. As expected, all Hg²⁺ transporting from the feed half-cell across the membrane was measured in the receiving half-cell rather than captured in the membrane. Open diamonds correspond to feed half-cell concentrations, while closed diamonds correspond to receiving half-cell concentrations.

FIG. 25 shows a plot obtained from electrodialysis of synthetic brackish water containing ˜5 ppm Hg²⁺ using a neat sPSF membrane; 7.5-mL half-cells were used, and −4 V vs. Ag/AgCl were applied across the cell. As expected, all Hg²⁺ transporting from the feed half-cell across the membrane was measured in the receiving half-cell rather than captured in the membrane. Open diamonds correspond to feed half-cell concentrations, while closed diamonds correspond to receiving half-cell concentrations.

FIG. 26 shows a plot obtained from electrodialysis of synthetic industrial wastewater containing ˜5 ppm Hg²⁺ using a neat sPSF membrane; 7.5-mL half-cells were used, and −4 V vs. Ag/AgCl were applied across the cell. As expected, all Hg²⁺ transporting from the feed half-cell across the membrane was measured in the receiving half-cell rather than captured in the membrane. Open diamonds correspond to feed half-cell concentrations, while closed diamonds correspond to receiving half-cell concentrations.

FIG. 27 shows Hg²⁺-capture electrodialysis of synthetic groundwater containing ˜5 ppm Hg²⁺ using 20 wt % PAF-1-SH membranes, with the x-axis representing mg of Hg²⁺ captured per dry g of PAF-1-SH in the membrane. Adsorption capacities (x-axis) were calculated using Eq. S5, based on the concentration of Hg²⁺ decreased in the feed half-cell. Volume changes in both half-cells due to removed sample aliquots and added HNO₃ and LiOH for OH⁻ and H⁺ neutralization, respectively, were included in the calculations; 7.5-mL half-cells were used, and −4 V vs. Ag/AgCl were applied across the cell.

FIG. 28 provides cconcentration profiles of competing cations in the Hg²⁺-capture electrodialysis of 5 ppm Hg²⁺ spiked in synthetic groundwater, using a 20 wt % PAF-1-SH in sPSF membrane. The concentration profiles for Hg²⁺ are included for comparison. No Hg²⁺ was detected in the feed solution after 2 h or longer of electrodialysis. Open and closed circles denote concentrations in the feed and receiving half-cells, respectively.

FIG. 29 shows a plot of Hg²⁺-capture electrodialysis of synthetic brackish water containing ˜5 ppm Hg²⁺ using 20 wt % PAF-1-SH membranes, with the x-axis representing mg of Hg²⁺ captured per dry g of PAF-1-SH in the membrane. Adsorption capacities (x-axis) were calculated using Eq. S5, based on the concentration of Hg²⁺ decreased in the feed half-cell. Volume changes in both half-cells due to removed sample aliquots and added HNO₃ and LiOH for OH⁻ and H⁺ neutralization, respectively, were included in the calculations; 7.5-mL half-cells were used, and −4 V vs. Ag/AgCl were applied across the cell.

FIG. 30 shows concentration profiles of competing cations in the Hg²⁺-capture electrodialysis of 5 ppm Hg²⁺ spiked in synthetic brackish water, using a 20 wt % PAF-1-SH in sPSF membrane. The concentration profiles for Hg²⁺ are included for comparison. No He was detected in the feed solution after 16 h or longer of electrodialysis. Open and closed circles denote concentrations in the feed and receiving half-cells, respectively.

FIG. 31 shows Hg²⁺-capture electrodialysis of synthetic industrial wastewater containing ˜5 ppm Hg²⁺ using 20 wt % PAF-1-SH membranes, with the x-axis representing mg of Hg²⁺ captured per dry g of PAF-1-SH in the membrane. Adsorption capacities (x-axis) were calculated using Eq. S5, based on the concentration of Hg²⁺ decreased in the feed half-cell. Volume changes in both half-cells due to removed sample aliquots and added HNO₃ and LiOH for OH⁻ and H⁺ neutralization, respectively, were included in the calculations; 7.5-mL half-cells were used, and −4 V vs. Ag/AgCl were applied across the cell.

FIG. 32 shows concentration profiles of major competing cations in the Hg²⁺-capture electrodialysis of 5 ppm Hg²⁺ spiked in synthetic industrial wastewater, using a 20 wt % PAF-1-SH in sPSF membrane. The concentration profiles for Hg²⁺ are included for comparison. No Hg²⁺ was detected in the feed solution after 6 h or longer of electrodialysis. Open and closed circles denote concentrations in the feed and receiving half-cells, respectively.

FIG. 33 shows concentration profiles of heavy metal competing cations in the Hg²⁺-capture electrodialysis of 5 ppm Hg²⁺ spiked in synthetic industrial wastewater, using a 20 wt % PAF-1-SH in sPSF membrane. The concentration profiles for Hg²⁺ are included for comparison. No Hg²⁺ was detected in the feed solution after 6 h or longer of electrodialysis. Open and closed circles denote concentrations in the feed and receiving half-cells, respectively.

FIG. 34 shows raw electrodialysis breakthrough data of 100 ppm Hg²⁺ in 0.1 M NaNO₃ by a neat sulfonated polysulfone (sPSF) membrane. Hg²⁺ immediately permeated through the membrane (i.e., was measured in the receiving half-cell in the first collected sample at min). 45-mL half-cells were used to ensure breakthrough during the experiment, as these large half-cells hold larger amounts of ions and possess a higher ratio of the feed solution volume to membrane area compared to smaller cells (e.g., 7.5-mL half-cells or industrial setups). Open diamonds represent feed half-cell Hg²⁺ concentrations, while closed diamonds represent receiving half-cell Hg²⁺ concentrations. Error bars denote the range of concentrations obtained from measurements on two separate samples.

FIG. 35 shows raw electrodialysis breakthrough data of 100 ppm Hg²⁺ in 0.1 M NaNO₃ by a 10 wt % PAF-1-SH in sPSF membrane. Hg²⁺ permeated through the membrane rather than being captured (i.e., was measured in the receiving half-cell) after ˜2.7 h. 45-mL half-cells were used to ensure breakthrough during the experiment, as these large half-cells hold larger amounts of ions and possess a higher ratio of the feed solution volume to membrane area compared to smaller cells (e.g., 7.5-mL half-cells or industrial setups). Open circles represent feed half-cell Hg²⁺ concentrations, while closed circles represent receiving half-cell Hg²⁺ concentrations. Error bars denote the range of concentrations obtained from measurements on two separate samples.

FIG. 36 shows raw electrodialysis breakthrough data of 100 ppm Hg²⁺ in 0.1 M NaNO₃ by a 20 wt % PAF-1-SH in sPSF membrane. Hg²⁺ permeated through the membrane rather than being captured (i.e., was measured in the receiving half-cell) after ˜6 h. 45-mL half-cells were used to ensure breakthrough during the experiment, as these large half-cells hold larger amounts of ions and possess a higher ratio of the feed solution volume to membrane area compared to smaller cells (e.g., 7.5-mL half-cells or industrial setups). Open circles represent feed half-cell Hg²⁺ concentrations, while closed circles represent receiving half-cell Hg²⁺ concentrations. Error bars denote the range of concentrations obtained from measurements on two separate samples.

FIG. 37 shows data resulting from electrodialysis of M HEPES (pH=6.5) containing ˜5 ppm Cu²⁺ by a neat sulfonated polysulfone membrane; 7.5-mL half-cells were used, and −2 V vs. Ag/AgCl were applied across the cell. As expected, Cu²⁺ transporting from the feed half-cell across the membrane was measured in the receiving half-cell rather than captured in the membrane. The final receiving Cu²⁺ concentration was slightly lower than the initial feed Cu²⁺ concentration likely due to ion exchange with the membrane, as ion exchangers typically exhibit slight selectivity of larger, multivalent ions (e.g., Cu²⁺) over competing ions in the solution (Na⁺). Open diamonds correspond to feed half-cell concentrations, while closed diamonds correspond to receiving half-cell concentrations.

FIG. 38 shows data resulting from electrodialysis of 0.1 M HEPES (pH=3) containing ˜2.3 ppm Fe³⁺ by a neat sulfonated polysulfone membrane; 7.5-mL half-cells were used, and −1.5 V vs. Ag/AgCl were applied across the cell. As expected, Fe³⁺ transporting from the feed half-cell across the membrane was measured in the receiving half-cell rather than captured in the membrane. The final Fe³⁺ concentrations were slightly lower than the initial feed Fe³⁺ concentration likely due to ion exchange with the membrane, as ion exchangers typically exhibit slight selectivity of larger, multivalent ions (e.g., Fe³⁺) over competing ions in the solution (Na⁺). Open diamonds correspond to feed half-cell concentrations, while closed diamonds correspond to receiving half-cell concentrations.

FIG. 39 shows data from Cu²⁺-capture electrodialysis using 20 wt % PAF-1-SMe membranes, with the x-axis representing mg of target ion captured per dry g of PAF in the membrane. Adsorption capacities (x-axis) were calculated using Eq. S5, based on the concentration of Cu²⁺ decreased in the feed half-cell. Volume changes in both half-cells due to removed sample aliquots were included in the calculations. Error bars denote the range of concentrations and adsorption capacities obtained from measurements on two separate samples. Applied voltage: −2 V vs. Ag/AgCl. Aqueous media: 0.1 M HEPES (pH=6.5). Half-cell volumes: 7.5 mL.

FIG. 40 shows data from Fe³⁺-capture electrodialysis using 20 wt % PAF-1-ET membranes, with the x-axis representing mg of target ion captured per dry g of PAF in the membrane. Adsorption capacities (x-axis) were calculated using Eq. S5, based on the concentration of Fe³⁺ decreased in the feed half-cell. Volume changes in both half-cells due to removed sample aliquots were included in the calculations. Error bars denote the range of concentrations and adsorption capacities obtained from measurements on two separate samples. Applied voltage: −1.5 V vs. Ag/AgCl. Aqueous media: 0.1 M HEPES (pH=3). Half-cell volumes: 7.5 mL.

FIG. 41 shows data from B(OH)₃-capture diffusion dialysis using 20 wt % PAF-1-NMDG membranes, with the x-axis representing mg of B(OH)₃ captured per dry g of PAF-1-NMDG in the membrane. Adsorption capacities (x-axis) were calculated using Eq. S5, based on the concentration of B(OH)₃ decreased in the feed half-cell. Volume changes in both half-cells due to removed sample aliquots were included in the calculations. No appreciable boric acid capture was observed when using neat sPSF membranes (FIG. 4C inset). Error bars denote the range of concentrations and adsorption capacities obtained from measurements on two separate samples. No external electric field was applied. Aqueous media in the feed half-cell: synthetic groundwater. Half-cell volumes: 1.7 mL.

FIG. 42 shows data from Hg²⁺-capture diffusion dialysis of a 0.1 M NaNO₃ solution containing 100 ppm Hg²⁺. All Hg²⁺ transporting from the feed half-cell into the Hg²⁺-selective PAF-1-SH membrane was captured, as no Hg²⁺ was detected in the receiving half-cell. This result suggests that selective capture of target species can be achieved in processes without an applied electric field, using adsorbent-based membranes. Open and closed points represent feed and receiving half-cell concentration, respectively. Red diamonds correspond to data from a neat sPSF membrane, and blue circles correspond to data from a 20 wt % PAF-1-SH in sPSF membrane. Half-cell volumes: 45 mL.

FIG. 43 shows that larger half-cell volumes (top: 45 mL; bottom: 7.5 mL) for a fixed membrane sample lead to drastically longer electrodialysis experimental times required. We note that the relatively long durations of all electrodialysis experiments in this work are mainly a result of the electrodialysis cell design rather than the membrane materials used, as the half-cell volume to membrane area ratios used in these experiments are drastically larger than those used in the membrane stack-spacer design in real industrial processes (71). A Nafion-115 (Chemours, 127 μm thickness, Na⁺ counterion form) membrane was used as the control membrane material. A synthetic groundwater solution spiked with Hg(NO₃)₂ (˜4.5 ppm Hg²⁺) was used as the initial feed solution, while 1 mM HNO₃ in DI water was used as the initial receiving solution. Applied voltage: −2 V vs. Ag/AgCl.

FIG. 44 shows results from ion-capture electrodialysis of synthetic groundwater containing ˜5 ppm Hg²⁺ using an electrodialysis stack. A membrane consisting of 20 wt % PAF-1-SH in sPSF was used as the cation exchange membrane, while a commercial Fumasep FAS-50 membrane was used as the anion exchange membrane. All Hg²⁺ was selectively captured from the feed (open circles) without detectable permeation into the cation receiving solution (closed circles). (Inset) All other cations were transported across the 20 wt % PAF-1-SH membrane to desalinate the feed. The feed desalination rate (>99%) was calculated using Eq. S11 and was determined based on the initial and final feed solution conductivities to account for both cation and anion removal. As expected, no Hg²⁺ or competing cations were detected in the anion receiving compartment at every collected aliquot throughout the duration of the experiment. Compartment volumes: 7.5 mL; applied voltage: 10 V.

FIG. 45 shows data from Hg²⁺-capture electrodialysis using an electrodialysis stack. A membrane consisting of 20 wt % PAF-1-SH in sPSF was used as the cation exchange membrane, while a commercial Fumasep FAS-50 membrane was used as the anion exchange membrane. Synthetic groundwater containing ˜5 ppm Hg²⁺ was used as the feed solution. The x-axis represents mg of Hg²⁺ captured per dry g of PAF-1-SH in the membrane. Adsorption capacities (x-axis) were calculated using Eq. S5, based on the change in concentration of Hg²⁺ in the feed compartment. No detectable Hg²⁺ was measured in the cation receiving or anion receiving compartments throughout the duration of the experiment. Volume changes in both half-cells due to removed sample aliquots were included in the calculations; 7.5-mL compartments were employed, and 10 V were applied across the cell.

FIG. 46 shows concentration profiles for competing cations in the ion-capture electrodialysis of 5 ppm Hg²⁺ spiked in synthetic groundwater, using a stack electrodialysis setup with a 20 wt % PAF-1-SH in sPSF membrane as the cation exchange membrane. The concentration profiles for Hg²⁺ are included for comparison. Open and closed circles denote concentrations in the feed and cation receiving compartments, respectively. No Hg²⁺ was detected in the feed solution after 2 h or longer of electrodialysis, and no Hg²⁺ or competing cations were detected in the anion receiving solution throughout the duration of the experiment.

FIG. 47 shows concentration profiles for Hg²⁺ and competing cations in the electrodialysis of 5 ppm Hg²⁺ spiked in synthetic groundwater. A stack electrodialysis setup was used with a neat sPSF cation exchange membrane and a Fumasep FAS-50 anion exchange membrane. As expected, nearly all Hg²⁺ transporting from the feed compartment (open diamonds) across the sPSF membrane was measured in the cation receiving solution (closed diamonds) rather than captured in the membrane. No measured cations were detected in the anion receiving solution throughout the duration of the experiment. Compartment volumes: 7.5 mL; applied voltage: 10 V.

FIG. 48 shows preliminary optimization results of membrane regeneration conditions. Five membrane samples consisting of 20 wt % PAF-1-SH in sPSF (˜10 mg) were first equilibrated in a 20 mL solution of 100 ppm Hg²⁺ in DI water to achieve Hg²⁺ adsorption. Desorption was then carried out using five different concentrated (12.1 M) HCl solutions with the indicated volumes. The percent Hg²⁺ desorbed in each case (blue bars) is compared with the result from the first regeneration cycle discussed in the main text (gray bar, see FIG. 2E). In the latter case, the membrane was washed with 20 mL of 12.1 M HCl followed by 20 mL of 2 M NaNO₃, and this process was repeated three times for a total regeneration solution volume of 160 mL. In each case, 100% of the captured Hg²⁺ was recovered. These results suggest that use of HCl alone and desorption volumes of 50 mL or less per g of membrane are needed to achieve complete desorption.

FIG. 49 indicates heightened proton conductivities are achieved with increased PAF loadings. These increased conductivities are enabled by the incorporation of high-diffusivity free volume pathways from the high-porosity PAFs. Conductivities were measured using a four-probe in-plane conductivity cell in a solution of DI water at ambient temperature and pressure, according to a previously reported protocol. Nyquist plots were generated for each sample using potentiostatic electrochemical impedance spectroscopy (see FIG. 18).

FIG. 50 provides a representative Nyquist plot used to calculate the ionic conductivity of each membrane type in the H′ counterion form. The AC voltage was varied about the open circuit potential at an amplitude of 80 mV using a Biologic SP-300 potentiostat and EC-Lab software. All data was collected using a frequency range of 0.5 MHz to 0.1 Hz and sampling 60 points per decade.

FIG. 51A-B provides a schematic illustration of ion-capture electrodialysis (IC-ED). (A) Upon applying an external electric field to trigger ion migration across ion-exchange membranes, (B) target ions (e.g., Hg²⁺) are selectively captured by adsorbents dispersed in the membranes. Simultaneously, common waterborne ions (e.g., Na⁺) permeate across the membranes to desalinate the feed and generate non-toxic brine solutions. The target ion is recovered for commodity re-use or proper disposal upon controlled release from the adsorbents. Though not shown, water splitting occurs at both electrodes to maintain electroneutrality, and the receiving solutions are often recycled before returned to the environment. Example adsorbents are shown with ion adsorption sites aligned along the interior of the adsorbent pores. Adsorption sites can also be appended directly to the membrane matrix. Analogous strategies can be applied to other existing membrane separations to capture target components from feed mixtures.

### DETAILED DESCRIPTION

As used herein and in the appended claims, the singular forms “a,” “an,” and “the” include plural referents unless the context clearly dictates otherwise. Thus, for example, reference to “a cell” includes a plurality of such cells and reference to “the fragment” includes reference to one or more fragments and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments or aspects only and is not intended to limit the scope of the present disclosure.

Other than in the operating examples, or where otherwise indicated, all numbers expressing quantities of ingredients or reaction conditions used herein should be understood as modified in all instances by the term “about.” The term “about” when used to described the present invention, in connection with percentages means ±1%. The term “about,” as used herein can mean within an acceptable error range for the particular value as determined by one of ordinary skill in the art, which can depend in part on how the value is measured or determined, e.g., the limitations of the measurement system. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. Also, where ranges and/or subranges of values are provided, the ranges and/or subranges can include the endpoints of the ranges and/or subranges. In some cases, variations can include an amount or concentration of 20%, 10%, 5%, 1%, 0.5%, or even 0.1% of the specified amount.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein an “absorbent” refers to a molecular entity that can effectively bind and separate from a mixture of molecular agents a desire agent. In certain embodiments, an absorbent is a porous particle. In another embodiment an absorbent is porous metal particles, porous metal oxide particles, metal organic framework (MOF) particles, a zeolitic organic framework (ZIF) particle, a covalent organic framework (COF) particle, and porous aromatic framework (PAF) particles. In certain embodiments, an absorbent is a porous aromatic framework (PAF) particle. In certain embodiments, an absorbent is functionalized to be selective for a particular molecular entity. In certain embodiments, the absorbent is functionalized with one or more functional groups selected from —NHR, —N(R)₂, NH(aryl), halides, aryl, aralkyl, alkenyl, alkynyl, pyridyl, bipyridyl, terpyridyl, anilino, —O(alkyl), cycloalkyl, cycloalkenyl, cycloalkynyl, sulfonamido, hydroxyl, cyano, —(CO)R, —(SO₂)R, —(CO₂)R, —SH, —S(alkyl), —SO₃H, —SO₃⁻M⁺, —COOH, COO⁻M⁺, —PO₃H₂, —PO₃H⁻M⁺, —PO₃H₂₋M²⁺, —CO₂H, silyl derivatives, borane derivatives, ferrocenes and other metallocenes, where M is a metal atom, and R is C₁₋₁₀ alkyl. In certain embodiments, the pore of a MOF, ZIF, COF, PAF is functionalized to contain the functional group.

As used herein a “fluid” refers to a liquid or gas. The fluid can be a multicomponent fluid containing a plurality of molecular entities.

As used herein a “membrane” refers to a permeable, selectively permeable or non-permeable film that can be used to divide or separate a first fluid from a second fluid.

The term “porous aromatic framework” or “PAF”, refers to a framework characterized by a rigid aromatic open-framework structure constructed by covalent bonds (Ben et al., 2009, Angew. Chem., Intl Ed. 48:9457; Ren et al., 2010, Chem. Commun. 46:291; Peng et al., 2011, Dalton Trans. 40:2720; Ben et al., 2011, Energy Environ. Sci. 4:3991; Ben et al., J. Mater. Chem. 21:18208; Ren et al., J. Mater. Chem. 21:10348; Yuan et al., 2011, J. Mater. Chem. 21:13498; Zhao et al., 2011, Chem. Commun. 47:6389; Ben & Qiu, 2012, Cryst Eng Comm, D01:10.1039/c2ce25409c). PAFs show high surface areas and excellent physicochemical stability, generally with long range orders and, to a certain extent, an amorphous nature. Porous aromatic frameworks lack the extended conjugation found in conjugated microporous polymers. A porous aromatic framework can have a surface area from about 50 m²/g to about 7,000 m²/g, about 80 m²/g to about 1,000 m²/g, 1,000 m²/g to about 6,000 m²/g, or about 1,500 m²/g to about 5,000 m²/g. A PAF can have a pore width of about 7 angstroms to about 30 angstroms (e.g., 10, 15, 20, 25 angstroms of any value between any of the foregoing). PAFs can have a differential pore volume of 0.02 to 0.30 cm³g⁻¹ Å⁻¹ (e.g., 0.02, 0.05, 0.15, 0.20, 0.25 cm³g⁻¹ Å⁻¹ of any value between any of the foregoing values).

The disclosure provides membrane composites comprising one or more selective absorbents for water purification, fuel cells, storage, ion-capture electrodialysis (IC-ED) and filtration.

An advantage of IC-ED over conventional ion-capture technologies is its multifunctional separation capabilities. These multifunctional capabilities are unique compared to other ion-capture technologies, such as adsorption units. As such, IC-ED can be uniquely used to reduce the number of steps or units needed in conventional water treatment trains for decontamination and/or desalination. A second major advantage of IC-ED is that it exhibits exceptional and tunable ion-ion selectivities needed to isolate individual target species from water mixtures. These capabilities are seldom exhibited by other conventional technologies, including ion exchange resins, absorbers, membranes, precipitation or coagulation methods, charge-based separations, filtration units, and electroplating.

Conventional electrodialysis membranes are highly selective for counterions over co-ions, but do not exhibit high counterion-counterion selectivities needed for target ion isolation. Neat sulfonated polysulfone membranes are capable of water desalination but not selective transport or capture of specific ions. Commercial Nafion-115 cation exchange membranes tested in electrodialysis setups also exhibit non-selective transport behavior. Reverse osmosis membranes are designed to separate all ions from water in a pressure-driven process that leads to highly efficient desalination but not selective ion isolation. Membrane capacitive deionization is an adsorption-based water desalination process wherein ions are collected capacitively in the electrical double layers of polarized electrodes. However, this electrostatic adsorption mechanism leads to low adsorption selectivities between different ion types with similar charge. Hence, these three leading membrane processes cannot achieve the multifunctional separations or excellent ion-ion selectivity offered by ion-capture electrodialysis as described herein.

While the multifunctional, tunable, and selective behavior of IC-ED is promising for process intensification routes and target ion recovery, this process is also expected to offer significant advantages in contaminant sequestration and waste handling compared to other ion removal technologies. Because ion-capture electrodialysis can isolate individual ion types (e.g., Hg²⁺) from other similar ion types (e.g., other cations and heavy metals), isolated ions may potentially be recovered at high enough purity for reuse. Isolated ions can alternatively be disposed as concentrated single-component waste, an economically advantageous option because waste management costs can vary widely depending on the contaminant types present in the waste. For example, waste that contains mercury is especially expensive, and waste mixtures that contain mercury even at relatively low concentrations but are otherwise benign must be treated as mercury hazardous waste. In contrast to IC-ED, other ion removal technologies with lower ion-ion selectivities (e.g., ion exchangers or capacitive deionization) frequently contain a variety of contaminant types in their waste streams, preventing versatility in sequestration options. Other conventional ion removal methods like precipitation and coagulation also typically lead to relatively large amounts of toxic waste.

In conventional electrodialysis, reverse osmosis, and membrane capacitive deionization, most ionic contaminants present in the feed water source remain in the produced brine stream. These contaminants become environmental pollutants if the brine is returned to the environment, devalue the brine if the brine is used in other applications such as resource extraction, or must be removed with costly pretreatment or post-treatment units. These brine management issues in membrane-based desalination technologies are especially significant because huge volumes of brine are generated by these technologies (e.g., water recovery rates are typically only ˜50% in reverse osmosis). Ion-capture electrodialysis shows promise in completely circumventing these various issues related to reuse, waste handling, and sequestration that are encountered with conventional ion removal technologies.

Ion-exchange membranes are dense, semi-permeable membranes made up of polymers with fixed charges. As such, ion-exchange membranes selectively reject co-ions from transporting through the membrane while permitting the transport of counterions. As an example, cation-exchange membranes feature fixed anionic groups (e.g., sulfonates) that allow the transport of cations while electrostatically rejecting anions. This high selectivity between co-ions and counterions has motivated the use of ion-exchange membranes in numerous industrial applications, such as for water desalination, electrolysis, diffusion dialysis, fuel cell technologies, and membrane bioreactors. However, conventional charged membranes face an ion permeability-selectivity tradeoff, where higher swelling leads to a decrease in ion selectivity but enlarges free volume pathways to increase ion permeability and water uptake. Moreover, the relatively low chemical stability and pH stability of traditional charged membranes remain major challenges in their development.

The disclosure provides for composite membranes which overcome the limitations of charged membranes. The composite membranes of the disclosure are incorporated with tunable absorbents. In some embodiment, the composite membranes comprise porous aromatic frameworks (PAFs). PAFs possess a high-porosity, and have a diamondoid-like structure that comprise organic nodes covalently and irreversibly coupled to aromatic linkages. As a result, PAFs display exceptional hydrothermal and chemical stabilities, such as stability in boiling water, concentrated acids and bases, and organic solvents. Furthermore, PAFs comprise chemical compositions similar to those of polymer matrices. For example, the disclosure demonstrates that strong PAF-polymer interfacial interactions bestow improved stability and transport properties to charged membranes. In contrast, other highly tunable nanomaterial classes often lack stability in water and compatibility with polymer matrices due to inorganic parts, limiting their development for composite charged membranes.

A PAF can comprise an organic node linked together by linking ligands, wherein the series of nodes have a formula selected from Formula I or Formula II:

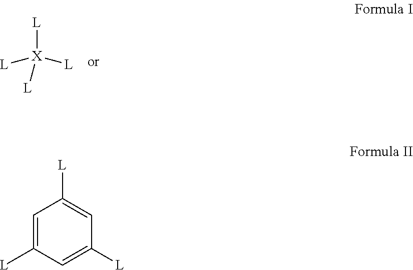

wherein, X is selected from C, B⁻ and P⁺; and L is a linking ligand;
and wherein the linking ligand has a structure of Formula III:

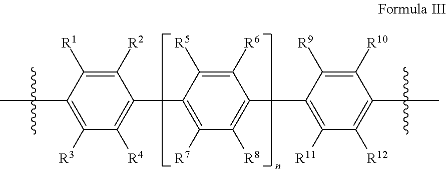

wherein, R¹-R¹² are independently selected from H, an optionally substituted (C₁-C₆)alkyl, an optionally substituted (C₁-C₆)alkenyl, an optionally substituted (C1-C5)-O-(C₁-C₆)alkyl, halo, —OH, —CH₂R¹³, —CO₂H, —COR¹⁴, —CO₂R¹⁴, —SH, —SMe, —SO₂H, —SO₃H, —NR¹⁵R¹⁶, —N⁺(H)₃, —N⁺(CH₃)₃, cyano, amide, azide, —PO₃H, —B(OR¹⁴)₂, 2-(methylthio)ethan-1-ol, N-methyl-D-glucamine, and heterocycle; R¹³ is selected from H, —OH, halo, —NH₂, —NR¹⁵R¹⁶, —N═C(CH₃)₂, -phthalimide, —C(NH₂)═N—OH, —SH, —SMe, —SO₂H, —SO₃H, —N⁺(H)₃, —N⁺(CH₃)₃, —PO₃H, cyano, amide, azide, —B(OR¹⁴)₂, - and heterocycle; R¹⁴ to R¹⁶ are independently selected from H or an optionally substituted (C₁-C₆)alkyl; and n is an integer selected from 0, 1, 2, 3, 4, or 5.

In certain embodiments, the composite comprises PAFs selected from the group consisting of PAF-1, PAF-1-CH₃, PAF-1-CH₂OH, PAF-1-CH₂-phthalimide, PAF-1-CH₂N═CMe₂, PAF-1-CH₂Cl, PAF-1-SH, PAF-1-ET (wherein ET is 2-(methylthio)ethan-1-ol), PAF-1-NMDG (wherein NMDG is N-methyl-D-glucamine), PAF-1-SMe, PAF-1-CH₂NH₂, and PAF-1-CH₂AO (wherein AO is an amidoxime group)

The disclosure provides a composite comprises a polymer/membrane matrix that contains or is embedded with one or more absorbents selected from metal organic frameworks (MOFs), covalent organic frameworks (COFs), zeolitic imidazolate frameworks (ZIFs), and/or porous aromatic frameworks (PAFs) that selectively binds to one or more targeted ions or organic molecules. In another or further embodiment, the polymer/membrane matrix comprises ion exchange polymer/membrane matrix materials. In one embodiment, the ion exchange polymer/membrane matrix materials is made from dimethyl-2-hydroxy benzyl amine, phenol and formaldehyde; C₆H₄ (OH)₂ or 1,2,3-C₆H₃(OH)₃, NH₂C₆H₄COOH, and formaldehyde; benzidine-formaldehyde and acrylonitrile-vinyl chloride copolymer; phenolsulfonic acid and formaldehyde; m-phenylene diamine or aliphatic diamine compounds and formaldehyde; tetrafluoroethylene and vinyl-ether; sulfonation and amination of styrene and divinylbenzene polymers; and sulfonated polysulfone. In one embodiment, the composite membrane contains from 5 wt % to 40 wt % of the one or more MOFs, COFS, ZIFs, and/or PAFs.

In one embodiment, the disclosure provides a composite anionic exchange membrane comprising a plurality of absorbents (e.g., a PAFs) that are selective for one or more anionic agents or anionic contaminants in a fluid stream. The absorbent may be uniformly distributed in the membrane or may be non-uniformly distributed. The plurality of absorbent may have a uniform pore size or a non-uniform pore size. By “uniform pore size” is meant that the pore size between two absorbents does not differ by more than 0.1%, 0.5% or 1%. In one embodiment, the anionic membrane contains from 5 wt % to 40 wt % of the one or more absorbents (e.g., MOFs, COFS, ZIFs, and/or PAFs).

In another embodiment, the disclosure provides a composite cationic exchange membrane comprising a plurality of absorbents (e.g., a PAFs) that are selective for one or more cationic agents or contaminants in a fluid stream. The absorbent may be uniformly distributed in the membrane or may be non-uniformly distributed. The plurality of absorbent may have a uniform pore size or a non-uniform pore size. In certain embodiments, the absorbent is a porous aromatic framework. In another embodiment, the composite cationic membrane is embedded with one or more metal organic frameworks (MOFs), covalent organic frameworks (COFs), zeolitic imidazolate frameworks (ZIFs), and/or porous aromatic frameworks (PAFs) that selectively binds to one or more targeted cationic molecules. In another or further embodiment, the polymer/membrane matrix comprises ion exchange polymer/membrane matrix materials. In one embodiment, the cationic exchange polymer/membrane matrix material is sulfonated polysulfone. In one embodiment, the cationic membrane contains from 5 wt % to 40 wt % (e.g., 10, 15, 20, 25, 30, 35 or 40 wt %) of the one or more MOFs, COFS, ZIFs, and/or PAFs. In one embodiment, the one or more PAFs are selected from PAF-1, PAF-1-CH₃, PAF-1-CH₂OH, PAF-1-CH₂-phthalimide, PAF-1-CH₂N═CMe₂, PAF-1-CH₂Cl, PAF-1-SH, PAF-1-ET, PAF-1-NMDG, PAF-1-SMe, PAF-1-CH₂NH₂, and PAF-1-CH₂AO (wherein AO is an amidoxime group). In a further embodiment, the one or more PAFs are selected from PAF-1-SH, PAF-1-ET, PAF-1-NMDG, PAF-1-SMe, PAF-1-CH₂NH₂, and PAF-1-CH₂AO. In still another or further embodiment, the composite cationic membrane selectively removes a targeted cationic agent selected from Hg²⁺, Nd³⁺, Cu²⁺, Pb²⁺, UO₂²⁺, B(OH)₃, Fe³⁺, and AuCl₄⁻.

The disclosure provides for composite membranes that have incorporated PAFs. The composite membranes of the disclosure have use in many possible applications, including for water treatment, ion-exchange, and electrochemical applications. Moreover, the composite membranes of the disclosure can be made to have specific selectivities for ions based upon the choice of incorporated PAFs. The disclosure demonstrates that PAFs, with altered pore morphologies and chemical affinities for specific ions, can be constructed and embedded into membranes through the rational choice of PAF node, linker, and linker-appended chemical functionality. Indeed, functionalized PAF variants have highest selectivities, kinetic rate constants, and capacities for capturing Hg²⁺, Nd³⁺, Cu²⁺, Pb²⁺, UO₂²⁺, B(OH)₃, Fe³⁺, or AuCl₄⁻ from water. The disclosure demonstrates that the exceptional adsorption performances of PAFs are retained upon incorporation into membrane matrices, thus, demonstrating the broad potential of PAF-incorporated charged membranes.

As described herein, any number of different adsorbents (e.g., PAFs) can be used in the compositions and methods of the disclosure. Dimensions of the gas passages, and hence the pressure drop through the membrane adsorbent bed, can be set by the characteristic dimension of the adsorbent (e.g., PAF), the density of adorbent packing, and the dispersity of the adsorbent sizes in addition to the membrane composition. The absorbent can be a relatively uniform density. In instances where the absorbent comprises a porous framework, the pore of the framework can be functionalized to be selective for a particular ionic charge or molecular size. In some embodiments, a plurality of differently functionalized PAFs or absorbents can be present in the membrane such that the membrane is selective for a plurality of different agents or contaminants in a fluid stream.

The adsorbent material can be selected according to the service needs, particularly the composition of the incoming fluid stream, the contaminants or agents which are to be removed and the desired service conditions, e.g., incoming gas pressure and temperature, desired product composition and pressure. Non-limiting examples of selective adsorbent materials can include, but are not limited to, microporous materials such as zeolites, metal organic frameworks (MOFs), AlPOs, SAPOs, ZIFs, (Zeolitic Imidazolate Framework based molecular sieves, such as ZIF-7, ZIF-8, ZIF-22, etc.), and carbons, as well as mesoporous materials such as amine-functionalized MCM materials, and combinations thereof.

Various membranes can be used in the methods and compositions of the disclosure and can be selected for their particular use and functionalized with an absorbent accordingly. Membranes suitable for use in the disclosed composites and fluid separation module include a metallic membrane such as palladium or vanadium. Alternative membrane embodiments are known to those skilled in the art, and generally comprise inorganic membranes, polymer membranes, carbon membranes, metallic membranes, composite membranes having more than one selective layer, and multi-layer systems employing non-selective supports with selective layer(s). Inorganic membranes may be comprised of zeolites, such as small pore zeolites, microporous zeolite-analogs such as AIPO's and SAPO's, clays, exfoliated clays, silicas and doped silicas. Inorganic membranes are typically employed at higher temperatures to minimize water adsorption. Polymeric membranes typically achieve hydrogen selective molecular sieving via control of polymer free volume, and thus are more typically effective at lower temperatures. Polymeric membranes may be comprised, for example, of rubbers, epoxies, polysulfones, polyimides, and other materials, and may include crosslinks and matrix fillers of non-permeable (e.g., dense clay) and permeable (e.g., zeolites) varieties to modify polymer properties. Carbon membranes are generally microporous and substantially graphitic layers of carbon prepared by pyrolysis of polymer membranes or hydrocarbon layers. Carbon membranes may include carbonaceous or inorganic fillers, and are generally applicable at both low and high temperature. Metallic membranes are most commonly comprised of palladium, but other metals, such as tantalum, vanadium, zirconium, and niobium are known to have high and selective hydrogen permeance. Metallic membranes typically have a temperature- and H₂-pressure-dependent phase transformation that limits operation to either high or low temperature, but alloying (e.g., with Copper) is employed to control the extent and temperature of the transition.

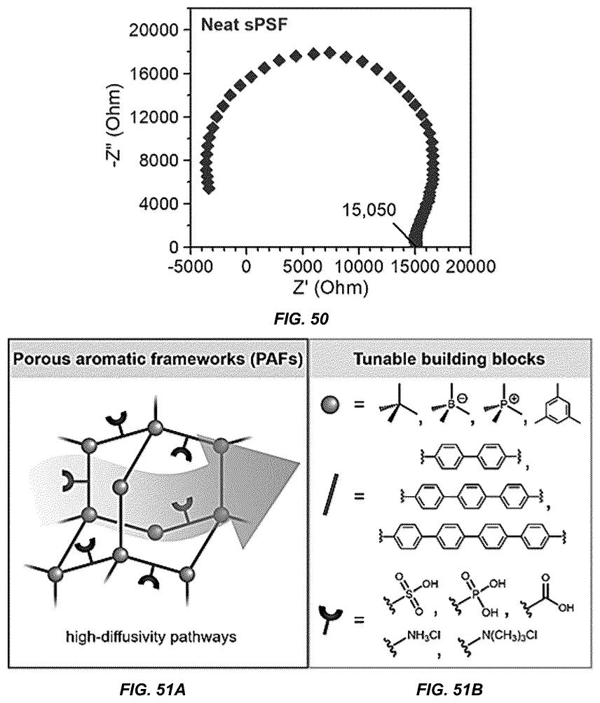

PAF-incorporated membranes advantageously exhibit an inverse effect to the typical permeability-selectivity tradeoff shown in conventional charged membranes. PAFs add porosity to the membranes to elevate their water uptake, and these high-diffusivity pathways in the PAF pores lead to heightened ion conductivities in PAF-embedded membranes compared to neat, conventional charged membranes (see FIGS. 49 and 50). However, while increased water uptake (and thus permeability) in charged membranes typically leads to increased swelling (and thus decreased selectivity), strong PAF-polymer crosslinking interactions diminish swelling in water. This reduced swelling prevents the formation of non-selective pathways in the polymer matrix.

This disclosure also provides a multifunctional, one-step separation method in which selective and tunable adsorbent particles or adsorption sites are incorporated into membranes (e.g., the composite membranes of the disclosure). In this approach, minor components of interest in a liquid- or gas-phase mixture are selectively captured by adsorption sites embedded in a membrane as the components transport through the membrane. Simultaneously, the feed stream is separated and purified via traditional membrane transport routes. The compositions and methods of the disclosure thus allow for the isolation of virtually any targeted component while simultaneously purifying the feed stream.

The selective separation of trace components of interest from various mixtures (e.g., micropollutants from groundwater, lithium or uranium from seawater, carbon dioxide from air) presents an especially pressing technological challenge. The composite membranes disclosed herein address existing drawbacks by providing highly selective and tunable adsorbents or adsorption sites which are embedded into membranes.

In a particular embodiment, the target species are selectively captured by the embedded adsorbents or adsorption sites of the composite membrane disclosed herein while the non-targeted species can either be transported or not-transported across the composite membrane. For example, in the exemplary experiments described herein, a composite membrane comprising incorporated Hg²⁺-selective adsorbents in an electrodialysis membrane provided for simultaneously capture of Hg²⁺ via an adsorption mechanism while desalinating water through an electrodialysis mechanism. Adsorption studies demonstrate that the embedded adsorbents maintain rapid, selective, regenerable, and high-capacity Hg²⁺ binding capabilities within the membrane matrix. Furthermore, when inserted into an electrodialysis setup, the composite membranes successfully capture all Hg²⁺ from various Hg²⁺-spiked water sources while permeating all other competing cations to simultaneously enable desalination. Finally, using an array of other ion-selective adsorbents, it was shown that other composite membranes could be produced which targeted a variety of ions that can be found in water sources. The composite membranes of the disclosure can be applied to existing membrane processes to efficiently capture targeted species of interest, without the need for additional expensive equipment or processes such as fixed-bed adsorption columns.

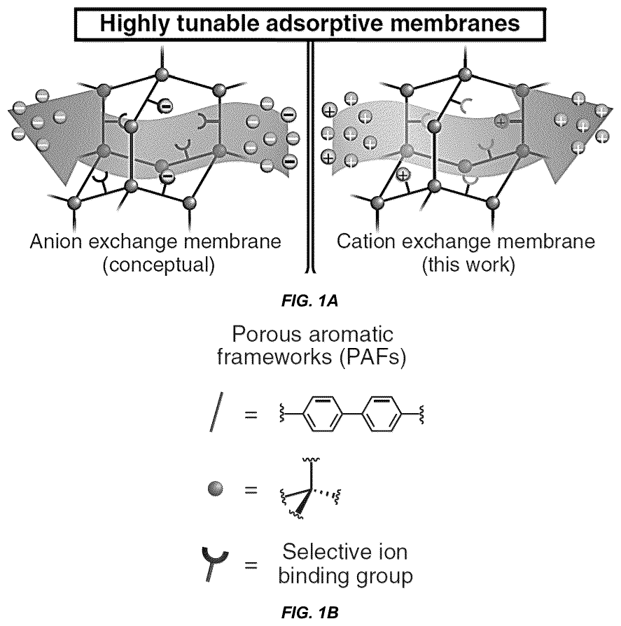

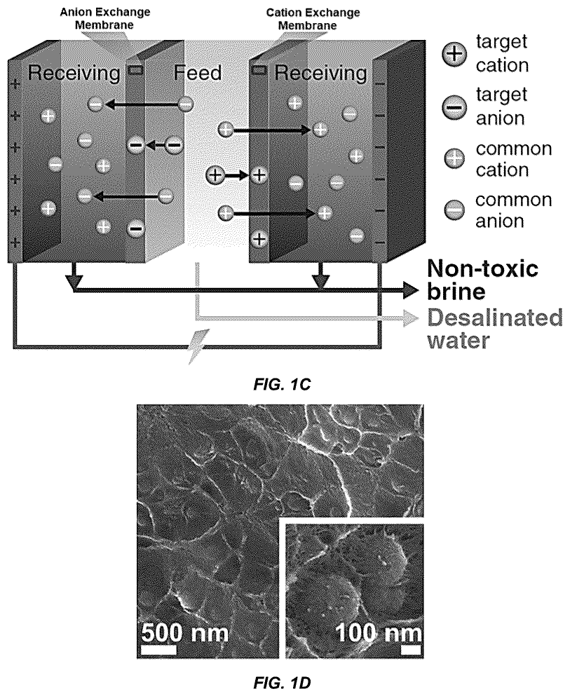

A schematic illustration of an ion-capture electrodialysis (IC-ED) design is depicted in FIG. 1C. As with conventional electrodialysis processes, an external voltage is applied to generate an electric potential gradient to drive cations and anions in the toxic, saline feed toward opposite directions. With selective cation-capture and anion-capture membranes placed in between the two electrodes in our system, competing ions permeate through the membranes freely to desalinate the feed, while target ions are captured by adsorbents dispersed in the membranes. Selective adsorption sites can also be grafted directly to the membrane matrix.

A system of the disclosure as set for in FIG. 1C can comprise (i) a composite anionic membrane comprising selective absorbents for anionic agents in a feed fluid stream, (ii) a composite cationic membrane comprising selective absorbents for cationic agents in a feed fluid stream, or (iii) both (i) and (ii).

The composite membranes of the disclosure can be used to (1) capture target ions as they permeate through a membrane, (2) desalinate and decontaminate feed water streams for reuse, and/or (3) obtain receiving solutions (e.g., brine) that are non-toxic. Moreover, the composite membranes of the disclosure can provide for all the foregoing in a simultaneous manner. Additionally, the disclosure provides for composite membranes in an adsorbent-based fluid separation membrane, the target molecule (e.g., mercury, sulfur compounds, carbon dioxide) is captured by selective binding sites, while the feed is simultaneously separated into retentate and permeate streams with permeate/retentate separation factors determined by the choice of membrane matrix material used. These goals are in conjunction with other variations of multifunctional separations described later in this disclosure that likewise utilize adsorbent-based membranes. For example, in an adsorbent-based gas separation membrane, the target molecule (e.g., mercury, sulfur compounds, carbon dioxide) is captured by selective binding sites, while the feed is simultaneously separated into retentate and permeate streams with permeate/retentate separation factors determined by the choice of membrane matrix material used.

While this approach can be used to capture any targeted anion or cation using high performance adsorbents selective for each given species, this was characterized using Hg²⁺, one of the most prevalent and toxic waterborne micropollutants, as a model target species. A Hg²⁺-selective porous aromatic framework functionalized with thiol groups (PAF-1-SH) was used as the model adsorbent and was dispersed in a sulfonated polysulfone (sPSF) cation conducting membrane matrix.

To assess the multifunctional IC-ED process for treating virtually any feed mixture, 20 wt % PAF-1-SH membranes were tested for the Hg²⁺-capture electrodialysis of 5 ppm Hg²⁺ spiked in synthetic groundwater, brackish water, and industrial wastewater. These feed sources were chosen for their diversity of salinity levels, ion types, and pH (Tables D and E). In these proof-of-concept experiments, a custom-made two-compartment cell was used, with the cation-capture membrane separating the feed from the “receiving” solution (10 mM HNO₃, to maintain conductivity and prevent metal precipitation). −4 V vs. Ag/AgCl were applied to drive feed cations through the membrane toward the receiving solution, and ion concentrations in both solutions were periodically measured. Remarkably, for each water source, Hg²⁺ was entirely captured by the adsorptive membranes, as Hg²⁺ was selectively reduced to concentrations below detection in the feed without permeating into the receiving solution. Meanwhile, all competing cations (Na+, K+, Mg²⁺, Ca²⁺, Ba²⁺, Mn²⁺, Fe³⁺, Ni²⁺, Cu²⁺, Zn²⁺, Cd²⁺, Pb²⁺) successfully transported into the receiving solution to achieve over 97-99% desalination of the feed. Desalination percentages were calculated based on the changes in the sum of the cation feed concentrations. No Hg²⁺ was captured when using conventional neat sPSF cation exchange membranes. These findings are summarized in Table G and highlight the unique and exceptionally selective multifunctional separation capabilities of an IC-ED method utilizing adsorptive membranes.

Breakthrough experiments were also conducted to reveal what percentage of embedded adsorption sites can be utilized in a multifunctional adsorbent-based membrane separation process. In these tests, a feed containing a high Hg²⁺ concentration (˜100 ppm) in a 0.1 M NaNO₃ supporting electrolyte was used along with a 1 mM HNO₃ receiving solution. Hg²⁺ concentrations were periodically tracked to identify the “breakthrough time” at which Hg²⁺ was first detected in the receiving solution instead of captured in the membrane. As expected, Hg²⁺ immediately permeated through a neat sPSF membrane without the adsorbent. Conversely, the breakthrough time by a 20 wt % PAF-1-SH membrane was approximately twice of that by a 10 wt % membrane, indicating high ion-capture efficiency. To quantitatively evaluate the percentage of membrane-embedded adsorbents that are utilized before breakthrough is reached in an IC-ED setup, the receiving Hg²⁺ concentrations were plotted against the amount of Hg²⁺ captured by PAFs in the membrane. Astonishingly, both the 10 wt % and 20 wt % PAF membranes experienced breakthrough after nearly all (97%) of the embedded adsorption sites were utilized, based on the Hg²⁺ equilibrium adsorption capacity attained by accessible PAF-1-SH powder at approximately equivalent testing conditions. These findings prove that high performance adsorption sites embedded in a membrane can be applied for the highly efficient and selective capture of target species when employed in a membrane separation process.

The disclosure is a generalizable and tunable approach applicable to virtually any target species. To validate this versatility, sPSF membranes were tuned to contain other high-performance adsorbents highly selective for other common waterborne contaminants (PAF-1-SMe10 for Cu2+ and PAF-1-ET11 for Fe3+). Membranes composed 20 wt % of PAF-1-SMe or PAF-1-ET were then tested in the IC-ED setup. Feed solutions of 6 ppm Cu2+ or 2.3 ppm Fe3+, respectively, in 0.1 M HEPES buffer (to supply competing ions and prevent precipitation upon OH— generation) were used. Excitingly, similar to in the Hg2+-capture electrodialysis tests, both membranes selectively captured their respective target ions entirely while achieving at least 96% desalination of the feeds to simultaneously produce reusable water. This ion capture behavior is absent when neat sPSF membranes without the adsorbents is used, highlighting the unique and highly selective transport properties of an adsorbent embedded membrane process.

To show that this can also be applied generally to other membrane processes, membranes were fabricated containing the B(OH)3-selective adsorbent PAF-1-NMDG. Membranes composed 20 wt % of PAF-1-NMDG in a sPSF matrix were placed in a diffusion dialysis setup without an applied electric field. Synthetic groundwater spiked with 4.5 ppm B(OH)3 was inserted into the feed half-cell, while the receiving half-cell was charged with deionized water. In these tests, a concentration gradient, rather than primarily an electric potential gradient, drove solute transport across the membrane. 20 wt % PAF-1-NMDG membranes completely captured B(OH)3 as it transported through the membrane, as B(OH)3 was reduced to concentrations below detection in the feed without any measured permeation into the receiving solution. No appreciable B(OH)3 was captured when a neat sPSF membrane was used. Membranes containing the Hg²⁺-selective PAF-1-SH also exhibited selective target species capture when employed in a solute-capture diffusion dialysis setup. Hence, the selective capture of various target species can be achieved by membranes containing selective adsorption sites regardless of the type of membrane separation process used, as different species transport driving forces can be applied.

For a general multifunctional adsorption-based membrane separation process to be effective, the following performance standards are suggested: (1) Binding groups must remain accessible within the membrane matrix. (2) Adsorbate binding rates must be faster than adsorbate transport rates through the membrane. (3) The adsorbent-based membrane must be regenerable such that adsorption sites are reusable and target adsorbates are recoverable. (4) The adsorbent-based membrane must possess sufficiently high selectivity toward the target adsorbates such that only the target adsorbates are captured. Competing species are not captured by the membrane and are instead rejected by or permeated through the membrane for purification of the inlet stream.

Through batch adsorption studies, each of these performance standards were indeed achieved by model adsorbent-based membranes consisting of PAF-1-SH embedded in sPSF. These studies can be used to predict the efficiency of adsorption sites incorporated in any membrane before use in a multifunctional membrane separation process.

To evaluate the first standard, membrane equilibrium adsorption isotherms were collected and compared to expected mass-averaged values based on the individual saturation adsorption capacities for the bare membrane matrix and adsorbent particles. In these experiments, neat sPSF membranes, PAF-1-SH bulk powder, and 20 wt % PAF-1-SH membranes were stirred until reaching equilibrium (at least 12 h for bulk PAF-1-SH or 48 h for membranes) in aqueous solutions containing varied initial Hg²⁺ concentrations. The initial and final concentrations were measured to extract equilibrium adsorption capacities. Based on the measured Hg²⁺ saturation capacity for the 20 wt % PAF-1-SH membranes compared to the theoretical maximum capacity, the percentage of PAF-1-SH adsorbent sites that remain accessible within the membrane matrix was determined to be as high as 93%.

To assess the second and third standards, adsorption kinetics and adsorption regeneration measurements were collected. In the kinetics studies, bulk PAF-1-SH was stirred in an aqueous solution containing 100 ppm Hg²⁺, and adsorption capacities were measured over time. These measurements indicate that the Hg²⁺ binding kinetics of the adsorbents are nearly instantaneous: over 81% of the Hg²⁺ saturation capacity is reached within the first 10 s of adsorption. In the regeneration studies, 20 wt % PAF-1-SH membranes were stirred for at least 48 h in an aqueous solution containing 100 ppm Hg²⁺. The membranes were then immersed in concentrated HCl followed by 2 M NaNO₃ to desorb and recover the captured Hg²⁺ while regenerating thiol adsorption groups in the membranes. After repeating these adsorption and desorption experiments over 10 cycles, only an 8% loss in Hg²⁺ capacity was observed, and the adsorption capacity remained approximately constant after the third cycle.

To investigate the fourth standard, equilibrium adsorption selectivity tests were performed. Bulk PAF-1-SH powder was stirred until reaching adsorption equilibrium in aqueous solutions of 100 ppm Hg²⁺ spiked in various prevalent water supply sources (groundwater, brackish water, industrial wastewater, or seawater; see Tables A and B). The initial and final concentrations of each solution were measured to obtain adsorption capacities. No loss in Hg²⁺ capacity was observed upon the presence of various abundant competing ions in each solution, indicating that the model PAF-1-SH adsorbents possess near-perfect multicomponent selectivity for Hg²⁺. These experiments were also repeated using membranes consisting of neat sPSF for comparison and 20 wt % PAF-1-SH. Ultra-high Hg²⁺ selectivity was preserved in the PAF-1-SH adsorption sites upon incorporation into a membrane polymer matrix, as the 20 wt % PAF-1-SH membranes achieved adsorption capacities matching those expected. Expected capacities were determined as the mass-averaged capacity based on the individual PAF-1-SH and sPSF adsorption uptakes. Results from these four performance standards indicate that the performance characteristics of adsorbents can be retained upon incorporation into membrane matrices, enabling their use in multifunctional adsorbent-based membrane separations as described in this disclosure.

The disclosure provides compositions and methods for selective capture of targeted components in any existing industrial process that uses membranes, provided that traditional membranes used in these processes are instead replaced with adsorbent-based composite membranes as described by the disclosure. Tunable multifunctional membrane of the disclosure can also obviate the need for additional industrial adsorption units, such as pressure swing adsorption or temperature swing adsorption technologies. Examples of potential applications and variations of the described disclosure include, but are not limited to, the following: (1) Selective recovery of targeted ions (e.g., organic ions, charged dyes, heavy metals, lithium, charged water pollutants) in liquid mixtures via charge-based separations. As provided herein, these separations can be achieved via ion-permeable membranes modified with adsorption sites or embedded with adsorbents that are selective for the targeted ions. Examples of traditional charge-based membrane separations in which adsorbent-embedded membranes can be implemented include electrodialysis, membrane capacitive deionization, and electrofiltration. In these cases, an electric potential gradient drives ion transport across the membrane, where target ions can then be captured. Water desalination can also be simultaneously achieved with selective ion recovery. (2) Using principles described herein, selective adsorbents can additionally be mixed directly into porous electrodes to capture target ions that transport into the electrodes. This approach could especially be effective in capacitive deionization separations to enable highly selective target ion recovery. In general, selective adsorption sites can be embedded into or onto various matrices (polymers, films, electrodes, etc.) through which the target component is permeable or to which the target ion contacts exposed adsorption sites on the surface of the matrix, to selectively capture the target component. (3) Selective recovery of charged or uncharged solutes using a solute-capture diffusion dialysis or solute-capture Donnan Dialysis approach implemented with adsorptive membranes. In this case, concentration gradients drive solute transport across the adsorptive membranes, where the target solute is selectively captured by adsorption sites incorporated in the membranes. (4) Selective capture of contaminants in fuel cell operations. For instance, these contaminants may be species like carbon monoxide or sulfur compounds that traditionally transport undesirably across the fuel cell membrane and subsequently poison the fuel cell catalyst. In accordance with the disclosure, membranes that contain adsorption sites selective for these contaminants (e.g., Nafion™ membranes embedded with selective adsorbents) may replace traditional contaminant-permeable membranes used in existing fuel cell operations (e.g., neat Nafion™ membranes). Using such adsorbent-based membranes, normal fuel cell operations can be performed while the contaminants are selectively captured concurrently. (5) Selective removal of contaminants in gas mixtures. For example, these contaminants may be species like mercury in coal flue gas mixtures or trace oxygen in inert gas mixtures. In accordance with disclosure, membranes that contain adsorption sites selective for these contaminants (e.g., membranes embedded with mercury-selective PAF-1-SH adsorbents) may act as a filter through which these gas mixtures transport to selectively capture the contaminants and permeate competing components. Such adsorbent-based membranes can also be applied in a multifunctional gas separation approach to replace traditional membranes used in gas separations. In this multifunctional approach, contaminants can be selectively captured as the feed gas mixture simultaneously separates into retentate and permeate streams with different compositions. In this case, contaminant selectivity in these composite membranes is dictated by the choice of embedded adsorption sites, while separation factors and permeabilities of the feed gas mixture are dictated by the choice of membrane polymer matrix. (6) Selective capture of CO₂ from the atmosphere. In accordance with this disclosure, membranes modified with strong CO₂-selective binding sites (e.g., amine- or polyamine appended, adsorbents) can act as a filter for direct air capture through which air is transported. During the transport of air through the adsorbent-based membrane, CO₂ in the air (present at a trace concentration of ˜410 ppm13) can be captured to yield a permeate stream with a reduced CO₂ concentration. CO₂ can then be recovered from the embedded adsorbents (e.g., via a temperature swing) for subsequent CO₂ utilization or sequestration. Similar strategies can be employed for the selective capture of other air pollutants (e.g., aldehydes) using adsorptive membranes selective for these pollutants. (7) Selective capture of dissolved CO₂ or CO₂-derived compounds (e.g., HCO₃⁻) from water. In accordance with this disclosure, membranes modified with strong CO₂-selective binding sites can be implemented for the capture of dissolved CO₂ or CO₂-derived compounds, which often undesirably alter solution pH and lead to ocean acidification. These CO₂-adsorbing membranes can be implemented into existing water treatment membrane processes (e.g., electrodialysis, reverse osmosis) or can be used as a filter through which aqueous solutions pass to exclusively capture the CO₂ compounds. When implemented into existing desalination technologies, the simultaneous desalination of water and capture of CO₂ or CO₂-derived compounds can be achieved within the same unit. (8) Selective capture and recovery of target compounds (e.g., contaminants or high-value compounds) in liquid mixtures using adsorbent-modified microfiltration, ultrafiltration, nanofiltration, or reverse osmosis membranes. In accordance with this disclosure, adsorbents or adsorption sites selective for these target compounds can be blended into any part of the membrane matrices, embedded into the membrane porous support layers, and/or grafted onto the top layer of the membrane (i.e., side of membrane active layer that faces the feed influent stream). As an example, adsorbents selective for boric acid, a common seawater pollutant that desalination membranes cannot efficiently reject, can be incorporated into reverse osmosis membranes for the simultaneous desalination of water and removal of boron in the same unit. Other examples of target compounds that adsorbent-based filtration membranes, unlike traditional filtration membranes, can be used for include pharmaceuticals, viruses, neutral organic micropollutants, small molecules in liquid fuel or organic solvent streams, and undesirable isomers in isomeric mixtures. Drug purification processes used in the pharmaceutical industry can also utilize adsorbent-based membranes innovated in this invention to obviate the need for other column purification units. (9) Selective removal of toxins from blood. In accordance with this disclosure, adsorbents or adsorption sites selective for these toxins can be blended into hemodialysis (i.e., blood dialysis) membranes, embedded into the membrane porous support layers, and/or grafted onto the top layer of the membranes. In this design, blood can be purified without the typical release of toxins into the dialysate solution, potentially allowing the dialysate to be recycled rather than disposed. Similar adsorbent-based membranes can also be applied as a filter through which contaminated blood solutions (e.g., from individuals with blood poisoning) transport to selectively remove the toxins from blood. (10) Selective capture of target compounds in organic liquid mixtures using adsorbent modified pervaporation or membrane distillation membranes. In accordance with this disclosure, adsorbents or adsorption sites selective for these target compounds can be blended into any part of the membrane matrices, embedded into the membrane porous support layers, and/or grafted onto the top layer of the membrane. Unlike in traditional pervaporation or membrane distillation processes, multifunctional separations utilizing adsorbent-based membranes can be achieved in which target compounds are captured while the feed mixture, following conventional pervaporation and membrane distillation principles, is separated into retentate and permeate mixtures with different desirable compositions. (11) As a variation to the materials and processes innovated by this disclosure, membranes with tunable catalytic sites, rather than tunable adsorption sites, can be developed using principles created in this invention. In this case, catalytic particles or reactive sites can be embedded into or appended onto a membrane matrix to create reactive membranes. In accordance with this disclosure, such reactive membranes can be used for the simultaneous separation of a feed mixture and conversion of a target component into a more desirable product. This desirable product can either be isolated following desorption from the membrane or can permeate through the membrane directly after conversion. Reactive membranes can also be applied for general catalytic applications. (12) The compositions and methods of the disclosure can also be used as a pretreatment or post-treatment step in various industrial processes, to partially or completely reduce the concentration of targeted components from mixtures. For example, this invention can be used to selectively recover nutrients from streams in a wastewater treatment plant or high-value components from brine effluent streams in a reverse osmosis plant. (13) This disclosure can additionally be applied as a replacement unit to existing fixed-bed adsorption columns for improved separations. While fixed-bed adsorption processes are a mature and developed technology, membrane separations are often more energy efficient and may possess fewer mass transfer limitations for improved separation selectivities. (14) As an analogous variation to the adsorbent-based membranes described in this disclosure, multiple different types of selective adsorbents or adsorption sites can be incorporated into the same membrane. Accordingly, these membranes can be used to capture multiple different target components within the same membrane. Similarly, multiple adsorbent based membranes selective for different target components can be placed sequentially in a multi-stage process, such as placed side-by-side in the same electrodialysis stack, to capture each target component in a stepwise fashion.

The composite membranes disclosed herein can be used as cation- or anion-exchange membranes or bipolar membranes used for water purification or water desalination. In this context, electrodialysis, Donnan Dialysis, and membrane capacitive deionization are three example technologies in which charged membranes incorporated with MOFs, COFs, ZIFs and/or PAFscan be used to achieve improved separation performances compared to those by conventional membranes. The composite membranes of the disclosure may also be used for other applications of these technologies, such as in the food processing industry.

The composite membranes disclosed herein can be used as fuel cell membranes (e.g., proton- or hydroxide-exchange membranes) with improved performance and stability compared to conventional neat membranes. The composite membranes as described herein may be used in place of traditional fuel cell membranes, to increase chemical stability (e.g., in organic solvents), pH stability, thermal stability, dimensional stability (i.e., swelling resistance), ion conductivity, and ion-exchange capacities.

The composite membranes disclosed herein can be used as reverse electrodialysis membranes for blue energy harvesting. In this technology, charged membranes are placed between a high-salinity aqueous solution (e.g., seawater) and a low-salinity aqueous solution (e.g., river water). These salinity gradients across the membranes generate an electrochemical potential difference that can be harvested as energy (“blue energy”). Previously described improvements achieved by the composite membranes disclosed herein compared to conventional membranes, such as decreased ionic resistance, may be exploited for this application.

The composite membranes disclosed herein can be used as charged membranes used for other general electrochemical applications that utilize a membrane, such as flow batteries. Previously described improvements achieved by the composite membranes disclosed herein compared to conventional membranes may be exploited for various electrochemical applications.

The composite membranes disclosed herein can be used as charged membranes used for selective ion separations. For example, PAFs can be incorporated into monovalent-selective polymer matrices to achieve improved separation performances for monovalent ions (e.g., Li⁺) over other ions. Additionally, the composite membranes of the disclosure can be tuned to create targeted pore sizes that enable molecular sieving can be incorporated into charged membranes to enhance molecular selectivity.

The composite membranes disclosed herein can be used as adsorptive membranes selective for targeted molecules, such as contaminants or high-value ions in water. PAFs selective for various waterborne species, as previously discussed, can be loaded into membranes to increase the capacity and selectivity for these species in the composite membranes of the disclosure. The selectivity of the composite membranes of the disclosure can be tuned according to the functional group and pore environment of the chosen MOFs, COFs, ZIFs and/or PAFs. Such adsorptive membranes can be used in place of adsorption columns, membrane adsorbers, or other adsorption technologies.

The composite membranes can be used for selective recovery of targeted ions (e.g., organic ions, charged dyes, heavy metals, lithium, charged water pollutants) in liquid mixtures via charge-based separations. As provided herein, these separations can be achieved via ion-permeable membranes modified with PAFs that are selective for the targeted ions. Examples of traditional charge-based membrane separations in which PAF-embedded membranes can be implemented include electrodialysis, membrane capacitive deionization, and electrofiltration. In these cases, an electric potential gradient drives ion transport across the membrane, where target ions can then be captured. Water desalination can also be simultaneously achieved with selective ion recovery.

Using the techniques described herein, selective MOFs, COFs, ZIFs and/and/or PAFs can additionally be mixed directly into porous electrodes to capture target ions that transport into the electrodes. This approach could especially be effective in capacitive deionization separations to enable highly selective target ion recovery. In general, selective MOFs, COFs, ZIFs, and/or PAFs can be embedded into or onto various matrices (polymers, films, electrodes, etc.) through which the target component is permeable or to which the target ion contacts exposed adsorption sites on the surface of the matrix, to selectively capture the target component.

The composite membranes disclosed herein can be used for selective capture of contaminants in fuel cell operations. For instance, these contaminants may be species like carbon monoxide or sulfur compounds that traditionally transport undesirably across the fuel cell membrane and subsequently poison the fuel cell catalyst. In accordance with this disclosure, composite membranes that contain MOFS, COFs, ZIFs and/or PAFs selective for these contaminants (e.g., Nafion™ membranes embedded with selective MOFS, COFs, ZIFs and/or PAFs) may replace traditional contaminant-permeable membranes used in existing fuel cell operations (e.g., neat Nafion™ membranes). Using such composite membranes, normal fuel cell operations can be performed while the contaminants are selectively captured concurrently.

The composite membranes disclosed herein can be used for selective removal of contaminants in gas mixtures. For example, these contaminants may be species like mercury in coal flue gas mixtures or trace oxygen in inert gas mixtures. In accordance with this disclosure, composite membranes that contain MOFS, COFs, ZIFs and/or PAFs selective for these contaminants (e.g., membranes embedded with mercury-selective MOFS, COFs, ZIFs and/or PAFs adsorbents) may act as a filter through which these gas mixtures transport to selectively capture the contaminants and permeate competing components. Such composite membranes can also be applied in a multifunctional gas separation approach to replace traditional membranes used in gas separations. In this multifunctional approach, contaminants can be selectively captured as the feed gas mixture simultaneously separates into retentate and permeate streams with different compositions. In this case, contaminant selectivity in these composite membranes is dictated by the choice of embedded MOFS, COFs, ZIFs and/or PAFs, while separation factors and permeabilities of the feed gas mixture are dictated by the choice of membrane polymer matrix.

The composite membranes disclosed herein can be used for selective capture of CO₂ from the atmosphere. In accordance with this disclosure, membranes modified with strong CO₂-selective MOFs, COFs, ZIFs and/or PAFs (e.g., amine- or polyamine functionalized frameworks) can act as a filter for direct air capture through which air is transported. During the transport of air through the composite membrane, CO₂ in the air (present at a trace concentration of ˜410 ppm) can be captured to yield a permeate stream with a reduced CO₂ concentration. CO₂ can then be recovered from the embedded MOFs, COFs, ZIFs and/or PAFs (e.g., via a temperature swing) for subsequent CO₂ utilization or sequestration. Similar strategies can be employed for the selective capture of other air pollutants (e.g., aldehydes) using composite membranes selective for these pollutants.

The composite membranes disclosed herein can be used for the selective capture of dissolved CO₂ or CO₂-derived compounds (e.g., HCO₃⁻) from water. In accordance with this disclosure, composite membranes comprising MOFs, COFs, ZIFs and/or PAFs that have strong CO₂-selective binding sites can be implemented for the capture of dissolved CO₂ or CO₂-derived compounds, which often undesirably alter solution pH and lead to ocean acidification. These composite membranes can be implemented into existing water treatment membrane processes (e.g., electrodialysis, reverse osmosis) or can be used as a filter through which aqueous solutions pass to exclusively capture the CO₂ compounds. When implemented into existing desalination technologies, the simultaneous desalination of water and capture of CO₂ or CO₂-derived compounds can be achieved within the same unit.

The composite membranes disclosed herein can be selective capture and recovery of target compounds (e.g., contaminants or high-value compounds) in liquid mixtures using a composite membrane as microfiltration, ultrafiltration, nanofiltration, or reverse osmosis membranes. In accordance with this disclosure, MOFs, COFs, ZIFs and/or PAFs selective for these target compounds can be blended into any part of the membrane matrices, embedded into the membrane porous support layers, and/or grafted onto the top layer of the membrane (i.e., side of membrane active layer that faces the feed influent stream). As an example, MOFs, COFs, ZIFs and/or PAFs selective for boric acid, a common seawater pollutant that desalination membranes cannot efficiently reject, can be incorporated into reverse osmosis membranes for the simultaneous desalination of water and removal of boron in the same unit. Other examples of target compounds that adsorbent-based filtration membranes, unlike traditional filtration membranes, can be used for include pharmaceuticals, viruses, neutral organic micropollutants, small molecules in liquid fuel or organic solvent streams, and undesirable isomers in isomeric mixtures. Drug purification processes used in the pharmaceutical industry can also utilize composite membranes described herein to obviate the need for other column purification units.

The composite membranes disclosed herein can be selective removal of toxins from blood. In accordance with this disclosure, composite membranes comprising MOFs, COFs, ZIFs and/or PAFs selective for these toxins can be used as hemodialysis (i.e., blood dialysis) membranes, embedded into the membrane porous support layers, and/or grafted onto the top layer of the membranes. In this design, blood can be purified without the typical release of toxins into the dialysate solution, potentially allowing the dialysate to be recycled rather than disposed. Similar composite membranes can also be applied as a filter through which contaminated blood solutions (e.g., from individuals with blood poisoning) transport to selectively remove the toxins from blood.

The composite membranes disclosed herein can be selective capture of target compounds in organic liquid mixtures using MOF, COF, ZIF and/or PAF modified pervaporation or membrane distillation membranes. In accordance with this disclosure, MOFs, COFs, ZIFs and/or PAFs selective for these target compounds can be blended into any part of the membrane matrices, embedded into the membrane porous support layers, and/or grafted onto the top layer of the membrane. Unlike in traditional pervaporation or membrane distillation processes, multifunctional separations utilizing composite membranes can be achieved in which target compounds are captured while the feed mixture, following conventional pervaporation and membrane distillation principles, is separated into retentate and permeate mixtures with different desirable compositions.

As a variation to the materials and processes innovated by this disclosure, membranes with tunable catalytic sites, rather than tunable adsorption sites, can be developed using principles described herein. In this case, catalytic MOFs, COFs, ZIFs and/or PAFs can be embedded into or appended onto a membrane matrix to create catalytically active composite membranes. In accordance with this disclosure, such composite membranes can be used for the simultaneous separation of a feed mixture and conversion of a target component into a more desirable product. This desirable product can either be isolated following desorption from the membrane or can permeate through the membrane directly after conversion. Composite membranes can also be applied for general catalytic applications.

The composite membranes described herein can be used as a pretreatment or post-treatment step in various industrial processes, to partially or completely reduce the concentration of targeted components from mixtures. For example, the composite membranes can be used to selectively recover nutrients from streams in a wastewater treatment plant or high-value components from brine effluent streams in a reverse osmosis plant.

The composite membranes of the disclosure can be applied as a replacement unit to existing fixed-bed adsorption columns for improved separations. While fixed-bed adsorption processes are a mature and developed technology, membrane separations are often more energy efficient and may possess fewer mass transfer limitations for improved separation selectivities.

The composite membranes of the disclosure can incorporate various types of MOFs, COFs, ZIFs and/or PAFs, in addition to the PAFs exemplified herein. In this case, the PAFs may be synthesized through an irreversible coupling reaction using other organic nodes, aromatic linkers, or functionalized chemical appendages. Other examples of PAFs that can be used with the composite membranes of the disclosure, include but are not limited to, Scholl-coupled PAFs that are relatively inexpensive, PAFs or COFs with anionic borate nodes, or catalytic MOFs, COFs, ZIFs or PAFs. Charged frameworks (e.g., MOFs, COFs, ZIFs and/or PAFs with anionic borate nodes or appended with charged groups) can also be embedded into neutral membranes to create charged composite membranes as discussed in this disclosure.

The composite membranes of the disclosure may comprise different polymer matrices, in addition to the sulfonated polysulfone polymer matrix exemplified herein. Other examples, of polymer matrices that can be used with MOFs, COFs, ZIFs and/or PAFs disclosed herein include perfluorinated sulfonic-acid (PFSA) ionomers and sulfonated polystyrene. The composite membranes of the disclosure may also comprise polymer matrices composed of multiple different charged polymers (e.g., bipolar membranes or copolymers) with MOFs, COFs, ZIFs and/or PAFs to yield improved composite membrane properties.

The disclosure provides for composite membranes that can be applied generally to various technologies that use ion-exchange membranes, or to adsorption processes where composite membranes detailed herein can be applied as membrane adsorbents. The composite membranes described herein can be applied for the selective capture of targeted components in any existing industrial process that uses membranes, provided that traditional membranes used in these processes are instead replaced with the composite membranes described herein. The composite membrane of the disclosure can also obviate the need for additional industrial adsorption units, such as pressure swing adsorption or temperature swing adsorption technologies.

As described herein, any number of MOFs, COFs, ZIFs and/or PAFs can be used in the composite membranes and methods of the disclosure. Dimensions of the gas passages, and hence the pressure drop through the membrane adsorbent bed, can be set by the characteristic dimension of the MOFs, COFs, ZIFs and/or PAFs, the density of MOF, COF, ZIF and/or PAF packing, and the dispersity of the adsorbent sizes in addition to the membrane composition. The MOFs, COFs, ZIFs and/or PAFs can be a relatively uniform density.

The MOFs, COFs, ZIFs and/or PAFs can be selected according to the service needs, particularly the composition of the incoming fluid stream, the contaminants or agents which are to be removed and the desired service conditions, e.g., incoming gas pressure and temperature, desired product composition and pressure. Non-limiting examples of framework materials that can be incorporated into the composite membranes disclosed herein can include, but are not limited to, microporous materials such as zeolites, metal organic frameworks (MOFs), COFs, ZIFs, (ZIF based molecular sieves, such as ZIF-7, ZIF-8, ZIF-22, etc.), AlPOs, SAPOs; as well as mesoporous materials such as amine-functionalized MCM materials, and combinations thereof.

These possibilities hold promise for the development of a wide range of potential PAFs to be incorporated into charged membranes to tune adsorptive, transport, and physical properties of the composite membranes for numerous desired applications (see FIG. 51).

### Examples

Synthesis and membrane fabrication. Carbon, hydrogen, nitrogen, and sulfur elemental analyses were obtained from the Microanalytical Facility at the University of California, Berkeley using a PerkinElmer 2400 Series II combustion analyzer. All porous aromatic framework (PAF) syntheses were performed using Schlenk techniques under an argon atmosphere. Ultrapure deionized (DI) water (18.2 MΩ cm electrical resistivity and less than 5.4 ppb total organic carbon) from a Millipore RiOs system was used as the water source for all syntheses and experiments. All starting materials and reagents were purchased from Sigma-Aldrich, Alfa Aesar, or Acros Organics and used as received unless otherwise stated.

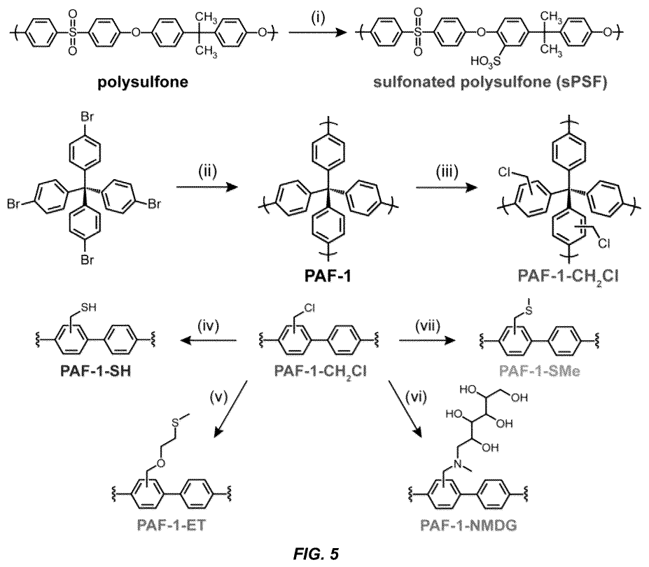

Synthesis of sulfonated polysulfone (sPSF) membrane matrix polymer. Sulfonated polysulfone (sPSF) was chosen as the cation exchange polymer matrix due to its extensive use in water purification applications. The reaction scheme for the sulfonation of polysulfone (PSF) is shown in FIG. 5. PSF resin (M_{W}=60,000) was first completely dried in a vacuum oven (24 h, 120° C.). In a 250-mL round-bottom flask, the dried resin (6 g) was completely dissolved in CHCl₃ (120 g, 80 mL). The mixture was capped with a rubber septum and lightly purged with desiccated N₂ for 10 min while stirring to remove moisture from the headspace. While vigorously stirring at room temperature, chlorosulfonic acid (750 μL) was slowly added dropwise using a glass syringe to immediately afford a deep pink precipitate. The capped mixture was vigorously stirred for 2.5 h and then poured into a 600-mL ice bath. After washing several times with DI water, the precipitate was collected and dried on a hot plate for min each at the following temperatures in succession: 60, 75, 90, 110° C. After each 30 min heating step, the solids were mechanically broken into small pieces for ease of handling. Finally, the sPSF was dried overnight in a vacuum oven at 80° C. to obtain ˜6.6 g of faint pink solids. The degree of sulfonation, defined here as sulfonate groups per PSF repeat unit, was found to be 60%. Using the above protocol, reactions were also carried out using different molar ratios of chlorosulfonic acid and dried PSF to verify that this procedure can be used to reproducibly control the degree of sulfonation.

Synthesis of PAF-1. The reaction scheme for the synthesis and post-synthetic functionalization of PAF-1 is displayed in FIG. 5. The monomer tetrakis(4-bromophenyl)methane was synthesized as a dark orange powder starting from triphenylmethyl chloride. Before use, column chromatography using SiO₂ (ROCC, 60 Å, 40-63 μm) and hexanes as the eluent was used to purify the monomer as a fluffy white powder before drying under vacuum overnight at 80° C.

A 500-mL two-neck Schlenk flask was charged with dried 2,2′-bipyridyl (1.1 g, 7.3 mmol), 1,5-cyclooctadiene (0.90 mL, 7.3 mmol), and anhydrous N,N-dimethylformamide (DMF, 110 mL) under an argon atmosphere. The sealed Schlenk flask was transferred to an Ar-purged glove tent, where bis(1,5-cyclooctadiene)nickel(0) (2.0 g, 7.3 mmol) was quickly added before a custom-made, air-free solid transfer adapter containing dried tetrakis(4-bromophenyl)methane (0.93 g, 1.5 mmol) was connected to the flask. The flask was resealed in the glove tent, and the solution was heated to 80° C. and stirred for 1.5 h to obtain a deep purple solution. The tetrakis(4-bromophenyl) methane was then slowly added to the solution under argon. The mixture was stirred at 80° C. for 16 h, after which the solution turned black. After slowly cooling to room temperature, the flask was opened to the air, and hydrochloric acid (6 M, 50 mL) was added dropwise. White solids slowly appeared in the solution toward the end of this addition. The solution was then stirred for 3 h under air and uncovered at room temperature, during which time the solution slowly changed into a turquoise color after ˜1 h. Failed synthesis attempts, possibly resulting from accidental air exposure before the final addition of acid, exhibited a darker, forest green color rather than a turquoise color. The turquoise solution was filtered, and the collected solids were washed with 250 mL each DMF, methanol, chloroform, dichloromethane, and tetrahydrofuran (THF) before dried overnight under vacuum at 180° C. to obtain ˜450 mg of PAF-1 as an off-white powder. PAF-1 ((C₂₅H₁₆)ₙ) elemental analysis: % calc. C94.9, H5.1; % found C94.4, H5.5.

Synthesis of PAF-1-CH₂Cl. Selective binding groups were appended post-synthetically onto PAF-1 via facile two-step reactions each starting with the chloromethylation of PAF-1 to PAF-1-CH₂Cl, which was performed as follows. PAF-1 (300 mg), paraformaldehyde (1.5 g), glacial acetic acid (9.0 mL), phosphoric acid (4.5 mL), and concentrated hydrochloric acid (12 M, 30 mL) were added to a 150-mL pressure vessel. The mixture was stirred for 3 d at 90° C. This mixture initially possesses a royal blue color that turns brown after ˜1 d of stirring. The solution was then filtered, and the solids were washed with methanol (1.0 L) and then dried overnight under vacuum at 110° C. to obtain ˜380 mg of PAF-1-CH₂Cl as a tan powder. PAF-1-CH₂Cl ((C₂₇H₂₀Cl₂)ₙ) elemental analysis: % calc. C78.1, H4.8, Cl17.1; % found C75.0, H4.7, Cl unmeasured. Degree of functionalization calculations for all functionalized PAFs based on elemental analyses are given in Table A.

TABLE A
Binding group loadings on the functionalized PAFs calculated
from elemental analysis results. Raw elemental analysis
results are provided in the Materials and Methods.

| | # | |
|---|---|---|
| | of functional | Functional |
| | groups per | group loading |
| Functionalized PAF | biphenyl linker | (mmol g⁻¹) |
| PAF-1-CH₂Cl ^{a} | 1.18 | 5.70 |
| PAF-1-SH ^{b} | 0.87 | 4.24 |
| PAF-1-SMe ^{b} | 1.00 | 4.58 |
| PAF-1-ET ^{b} | 0.45 | 1.72 |
| PAF-1-NMDG ^{c} | 0.95 | 2.60 |

^{a} Loadings for PAF-1-CH₂Cl were calculated using carbon elemental analysis results.
^{b} Loadings for PAF-1-SH, PAF-1-SMe, and PAF-1-ET were calculated using sulfur elemental analysis results. The relatively lower functional group loading in PAF-1-ET was also reported previously and is likely attributed to side products formed as a result of the reactivity of sodium hydride used in this functionalization reaction.
^{c} Loadings for PAF-1-NMDG were calculated using nitrogen elemental analysis results.

Synthesis of PAF-1-SH. The Hg²⁺-selective PAF-1-SH was synthesized as follows. Under argon, PAF-1-CH₂Cl (300 mg), sodium hydrosulfide (1.2 g), and ethanol (100 mL) were added to a 250-mL Schlenk flask and stirred under reflux for 3 d. The resulting solids were collected and washed with 250 mL each water and methanol and then dried overnight under vacuum at 110° C. to obtain ˜280 mg PAF-1-SH as a pale yellow powder. PAF-1-SH ((C₂₇H₂₂S₂)ₙ) elemental analysis: % calc. C79.0, H5.4, S15.6; % found C78.9, H5.6, S13.6.

Synthesis of PAF-1-SMe. The Cu²⁺-selective PAF-1-SMe was synthesized as follows. Under argon, PAF-1-CH₂Cl (300 mg), sodium thiomethoxide (1.2 g), and ethanol (100 mL) were added to a 250-mL Schlenk flask and stirred at 70° C. for 3 d. The resulting solids were then collected and washed with 100 mL each water, ethanol, chloroform, and THF and then dried overnight under vacuum at 120° C. to obtain ˜315 mg PAF-1-SMe as a light tan powder. PAF-1-SMe ((C₂₉H₂₆S₂)ₙ) elemental analysis: % calc. C79.4, H6.0, S14.6; % found C77.0, H6.0, S14.7.

Synthesis of PAF-1-ET. The Fe³⁺-selective PAF-1-ET was synthesized as follows. The PAF-1 precursor for PAF-1-ET was synthesized using tetrakis(4-bromophenyl)methane monomer purchased from TCI America. This monomer was dried overnight under vacuum at ° C. and otherwise used without further purification. Under argon, 2-(methylthio)ethanol (1.83 mL), NaH (60% dispersion in mineral oil, 1.5 g total), and anhydrous, degassed toluene (100 mL) were combined in a 250-mL Schlenk flask. After mixing for 5 min, PAF-1-CH₂Cl (260 mg) was added. The light brown mixture was stirred for 3 d at 90° C. The solution was then filtered, and the solids were washed with 100 mL each water, ethanol, chloroform, and THF and then dried overnight under vacuum at 150° C. PAF-1-ET ((C₃₃H₃₄O₂S₂)ₙ) elemental analysis: % calc. C 75.2, H 6.5, O 6.1, S 12.2; % found C 74.9, H 5.1, O unmeasured, S 5.5. The considerable discrepancy between the expected and observed sulfur elemental analysis, and thus functional group loading, was previously observed and is likely attributed to side reactions formed from the use of NaH.

Synthesis of PAF-1-NMDG. The B(OH)₃-selective PAF-1-NMDG was synthesized as follows. PAF-1 (300 mg), N-methyl-D-glucamine (NMDG, 12 g), and DMF (40.0 g, 42.4 mL) were added to a 150-mL pressure vessel. The light brown mixture was stirred for 3 d at 90° C. The solution was then filtered, and the solids were washed with methanol (1.5 L) and then dried overnight under vacuum at 120° C. to obtain ˜450 mg of PAF-1-NMDG as a light tan powder. PAF-1-NMDG ((C₄₁H₅₂N₂O₁₀)ₙ) elemental analysis: % calc. C67.2, H7.2, N3.8, O21.8; % found C65.3, H7.0, N3.6, O unmeasured.

Fabrication of composite membranes. Membranes were fabricated via a solvent evaporation approach. Separate solutions of 1 wt % PAF-1-R (R═SH, SMe, ET, or NMDG) in DMF and 10 wt % sPSF (60% sulfonation) in DMF were stirred overnight at ˜450 rpm. The PAF-1-R solution was then fully dispersed via sonication for 1 h before ˜20% of the sPSF solution was added dropwise to the PAF solution while stirring. This “priming” step is believed to promote interactions between the filler and polymer in composite materials by covering the filler with a thin polymer layer. The composite solution was mixed for 1 h at ˜600 rpm and then sonicated for 1 h before the remaining sPSF solution was added dropwise while stirring. The resulting solution was then mixed for 1 h at ˜600 rpm and then sonicated for 1 h. No individual PAF agglomerations could be visibly observed in the solution following these mixing and sonication steps. The dispersed solution was then casted into a homemade borosilicate glass dish before covered with a folded Kimwipe. DMF was slowly evaporated from the casted solution in a vacuum oven at ˜26 in Hg vacuum pressure (i.e., ˜4 in Hg absolute pressure), 60° C. for 16 h, and then 80° C. for 4 h to yield dense membrane films with ˜80±25 μm thickness as measured using a digital micrometer. The freestanding films were stored in DI water replaced at least twice daily for at least one week before use to remove residual solvent. Complete removal of DMF was confirmed via infrared spectroscopy and nitrogen elemental analysis. Accurate PAF loadings were confirmed via thermogravimetric analysis (TGA) decompositions of the fabricated membranes.

Neat sPSF membranes were fabricated using the same method but without the priming and PAF addition steps. PAF-1-NMDG composite membranes and sPSF membranes used in diffusion dialysis were prepared via the same protocol but using half the amounts of PAF and sPSF, such that these membranes were measured to have ˜40±10 μm thicknesses.

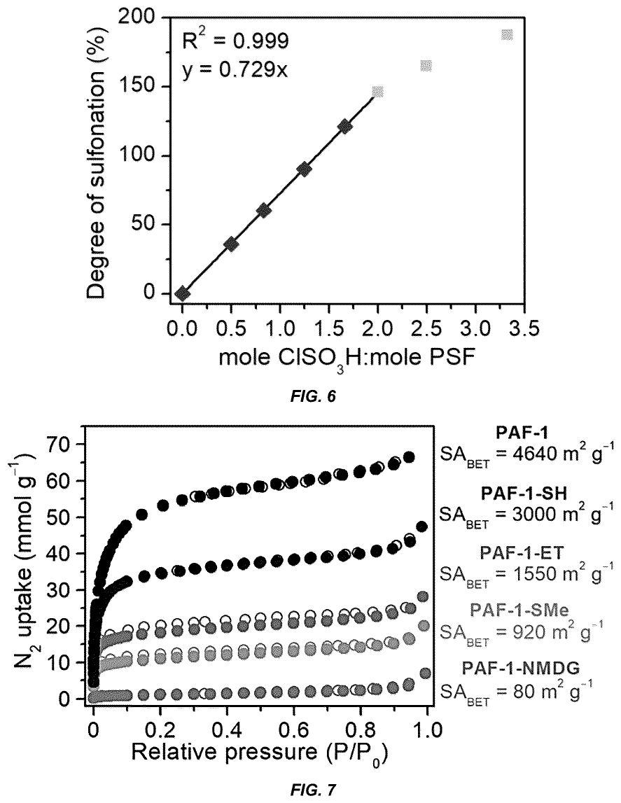

Degree of sulfonation calculations based on ¹H NMR. The degree of sulfonation, defined here as sulfonate groups per PSF repeat unit, was determined from ¹H NMR spectra and confirmed by acid-base titration. ¹H NMR spectra were collected on a 300 MHz Bruker Avance spectrometer and internally referenced to the residual solvent signals. Samples were prepared using PSF or sPSF resin dissolved completely in CDCl₃ or DMSO-d₆ (Cambridge Isotope Laboratories), respectively. The degree of sulfonation (DS) was calculated using Kopf's formula, given by:

$$DS = \frac{12 - 4 r}{2 + r} \qquad ( S 1 )$$

where r is the ratio of A_{abc}/A_{de}, A_{abc} is the combined integration of ¹H NMR peaks due to protons a, b, and c, and A de is the combined integration peaks due to protons d and e. The DS of the sPSF used in membrane samples was found to be ˜60%. The degrees of sulfonation calculated for sPSF samples synthesized using different ratios of chlorosulfonic acid to PSF are presented in FIG. 6 to demonstrate the precise control of DS by the synthetic protocol used.

To confirm the accessibility of sulfonate groups in the sPSF to ions, standard acid-base titration using phenolphthalein indicator was also performed on a sPSF membrane with DS=60%. The ion exchange capacity was found to be ˜1.1 mmol g⁻¹.

Material characterizations of porous aromatic framework (PAF) particles. Degree of functionalization calculations for all functionalized PAFs based on elemental analyses are given in Table A.

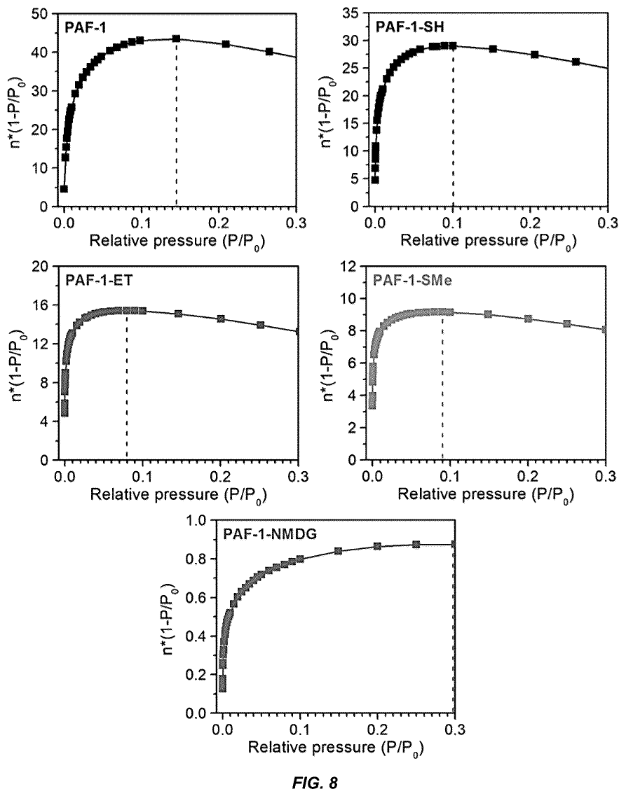

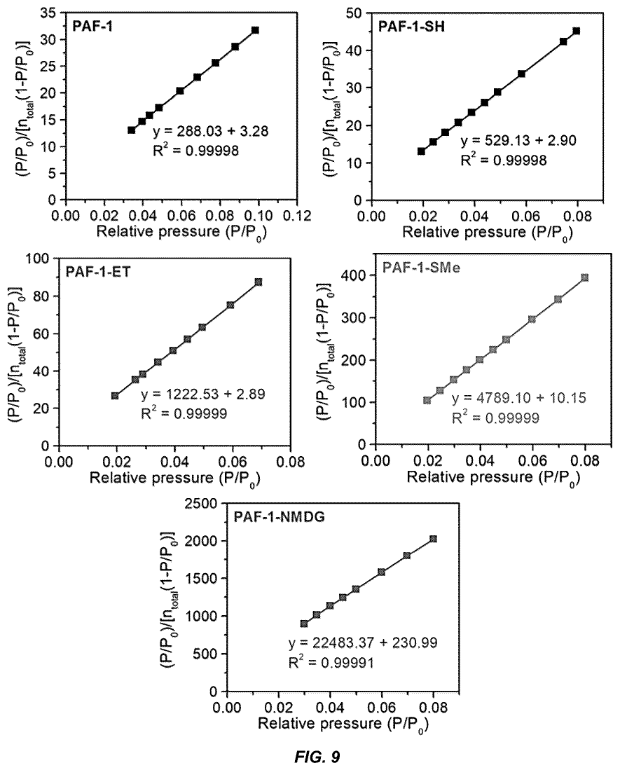

Surface area and pore size measurements. PAF surface areas were determined from N₂ adsorption isotherms obtained at 77 K using a Micromeritics ASAP 2420 instrument. Activated samples (˜70 mg) were transferred to a pre-weighed glass analysis tube capped with a TranSeal. Before gas adsorption analysis, the samples were evacuated ˜24 h on the ASAP 2420 instrument at the respective drying temperature of each PAF sample. Samples were considered fully activated once the outgas rate was less than 2 μbar min⁻¹, which occurred within this 24 h timeframe. Nitrogen adsorption isotherms (FIGS. 7-9) were obtained using ultra-high purity grade (99.999%) nitrogen and a 77 K liquid-N₂ bath, and a molecular cross-sectional area of 16.2 Å² was assumed for N₂.

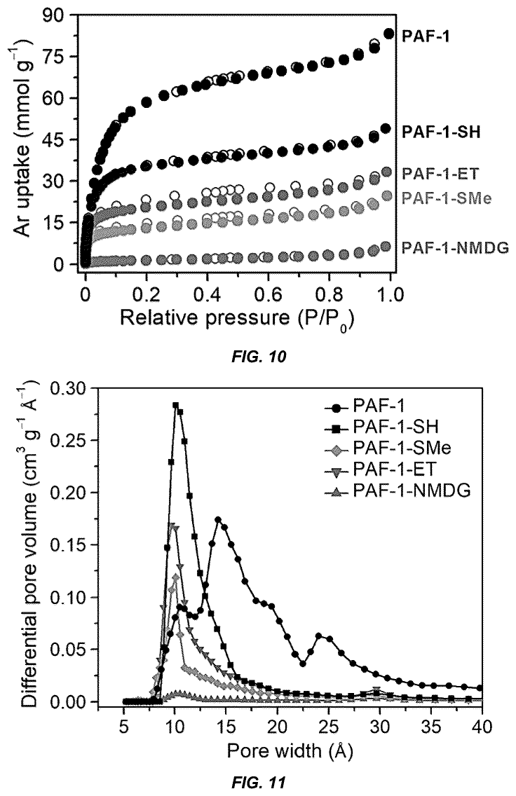

PAF pore size distributions were measured via argon adsorption isotherms (FIG. 10) at 87 K using otherwise identical methods to the nitrogen adsorption isotherm measurements. Ultra-high purity grade (99.999%) argon and an 87 K liquid-Ar bath was used, and a molecular cross-sectional area of 14.2 Å² was assumed for Ar. Pore size distributions (FIG. 11) were calculated from the adsorption branch of the 87 K Ar isotherms by the quenched solid density functional theory (QSDFT) method using a carbon-based material with a slit-pore model (Quantachrome QuadraWin Ver. 6.0). This model provided the best fits (<1% fitting error for each material) but may not most accurately reflect the actual pore geometries in the materials.

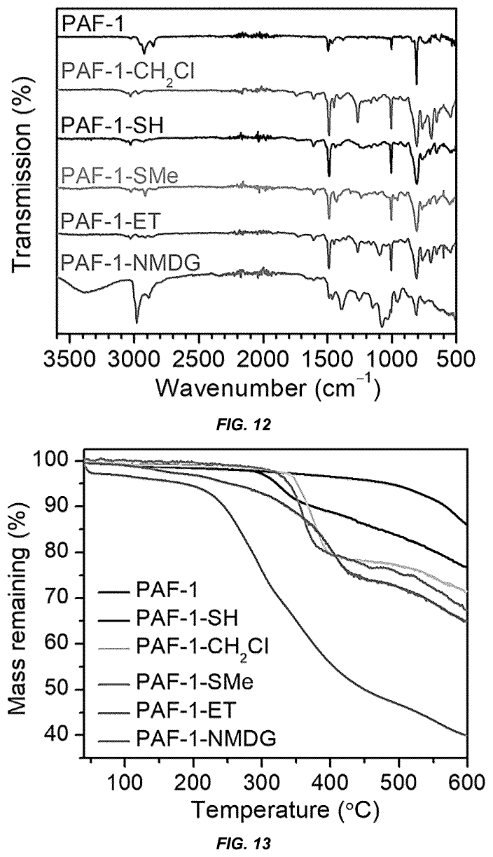

Fourier-transform infrared spectroscopy (FTIR). FTIR spectra (FIG. 12) were collected at ambient conditions on a PerkinElmer Spectrum 100 Optica FTIR spectrometer furnished with an attenuated total reflectance accessory.

Thermogravimetric analysis (TGA) decomposition. TGA data (FIG. 13) were recorded using a TA Instruments TGA Q5000 under flowing N₂ gas at a ramp rate of 5° C. min′.

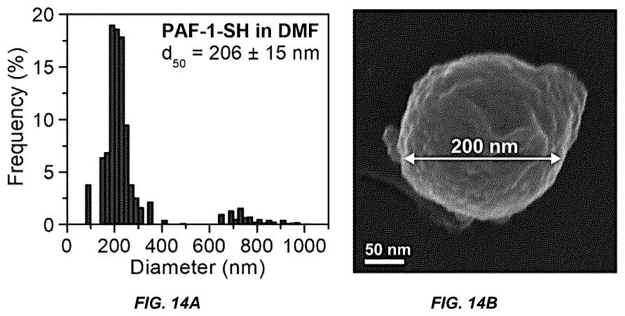

Particle size measurements using dynamic light scattering (DLS). Number-averaged PAF-1-SH particle size distributions (FIG. 14A) were measured using a Brookhaven BI-200SM DLS system at a 90° scattering angle. Samples were prepared by first stirring PAF-1-SH (˜0.25 mg) in DMF (˜4 mL) overnight at ˜450 rpm. The solution was then completely dispersed via sonication for 1 h before quickly performing DLS measurements at room temperature. A refractive index of 1.6 was assumed for the particles, and each data trial was collected over 60 s using a laser beam wavelength of 637 μm. The reported DLS data is compiled from 10 separate measurements.

Imaging PAFs via field emission scanning electron microscopy (FESEM). FESEM images (FIG. 14) were taken using a Hitachi S-5000 SEM at the Electron Microscope Laboratory at the University of California, Berkeley. PAF particle samples were prepared by dispersing the materials in methanol using otherwise similar protocols as used for DLS sample preparation. Dispersed PAF solutions were then drop casted onto silicon chips. Single particle images were collected using PAF solutions that were further diluted. To dissipate charge, the samples were sputter-coated with gold using a Tousimis sputter coater prior to imaging.

Material Characterizations of Fabricated Membranes

Confirming PAF loading via TGA decomposition. The loadings of PAF-1-SH in sPSF membranes were confirmed by a thermogravimetric analysis method (Table B), based on the higher thermal stability of PAF-1-SH than that of sPSF at high temperatures. Membrane samples immersed in DI water were dried overnight in a vacuum oven (80° C.) before being quickly transferred to a TA Instruments TGA Q5000 instrument. Under flowing N₂, the samples were then heated to 600° C. at ramp rates of 5° C. min⁻¹. PAF-1-SH loadings (x, wt %) were calculated based on the mass remaining of each composite membrane sample at 600° C. (MR_{composite}, %), which was compared to the individual masses remaining after TGA decomposition of PAF-1-SH powder (MR_{PAF}, %) and neat sPSF membrane (MR_{sPSF}, %) at 600° C., as shown in Eq. S2:

$$x = 100 \% \times ( \frac{{MR}_{composite} - {MR}_{sPSF}}{{MR}_{PAF} - {MR}_{sPSF}} ) \qquad ( S 2 )$$

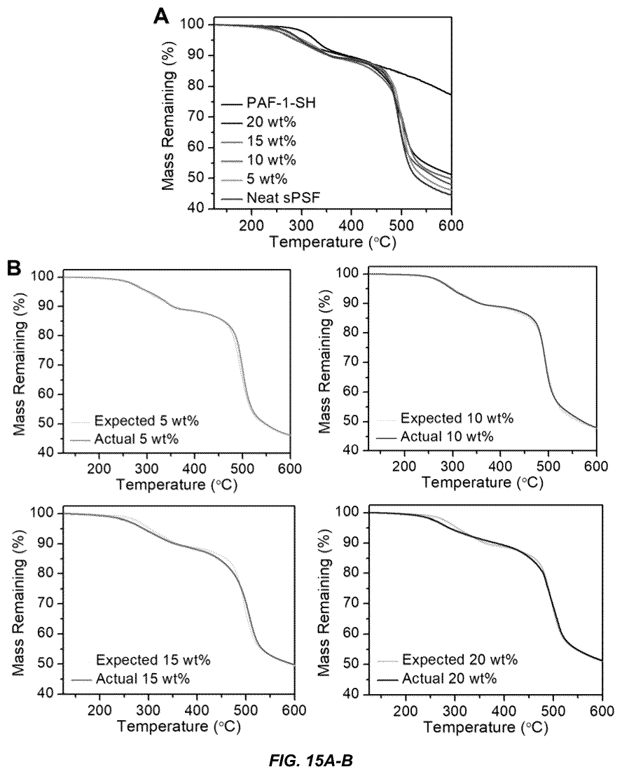

To account for any solvent (water) loss effects, the mass remaining at 125° C. was taken as 100%. TGA decomposition profiles and their comparisons to expected profiles are given in FIG. 15.

TABLE B
Comparison of theoretical PAF-1-SH loadings to observed
PAF-1-SH loadings in the fabricated composite membranes.

| Theoretical loading | Observed loading | Observed loading |
|---|---|---|
| (wt %) ^{a} | (wt %) ^{b} | (vol %) ^{c} |
| 5.0 | 4.9 | 14.1 |
| 10.0 | 10.4 | 27.0 |
| 15.0 | 15.9 | 37.6 |
| 20.0 | 20.0 | 44.3 |

^{a} Theoretical PAF-1-SH loadings are based on the relative masses of PAF-1-SH used compared to sPSF during membrane fabrication.
^{b} Observed PAF-1-SH wt % loadings were calculated from TGA decomposition results, based on the mass remaining in each membrane sample at 600° C.
^{c} Observed PAF-1-SH vol % loadings were calculated from helium pycnometry, the amount of N₂ gas adsorption uptake at P/P₀ = 0.98, and the observed wt % loadings.

Imaging PAF dispersibility through cross-sectional FESEM. FESEM images of membrane cross-sections were collected using a Hitachi S-5000 SEM at the Electron Microscope Laboratory at the University of California, Berkeley. Film cross-sections were exposed by fracturing in liquid nitrogen before sputter-coating with gold to dissipate charge. Cross-sectional images are shown in FIG. 1.

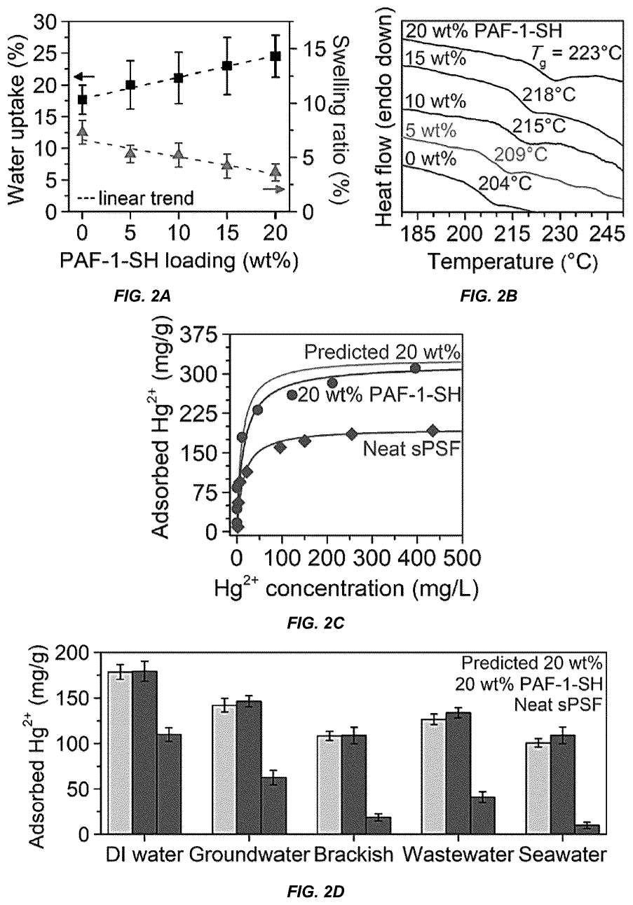

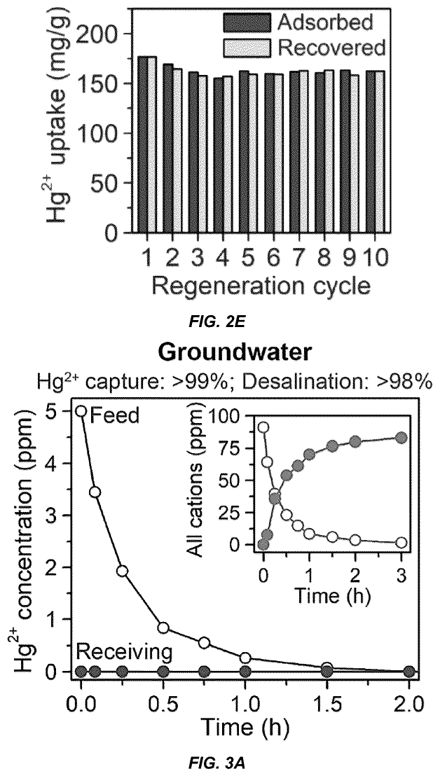

Determination of glass transition temperature (T_{g}). The glass transition temperature (T_{g}) for membranes fabricated using various functionalized PAFs (Table C) or with different PAF-1-SH loadings (FIG. 2B) was determined via differential scanning calorimetry using a TA Instruments Q200 instrument. A scan rate of 10° C. min⁻¹ was applied, and the second heating scan was taken for the T_{g}.

TABLE C
The glass transition temperature (T_{g}) for composite membranes
consisting of various functionalized PAFs incorporated in
sPSF, suggesting favorable interactions between the PAFs
and sPSF matrix regardless of PAF functional group.

| Membrane material | T_{g} (° C.) |
|---|---|
| Neat sPSF | 204 |
| 20 wt % PAF-1-SH | 223 |
| 20 wt % PAF-1-SMe | 218 |
| 20 wt % PAF-1-ET | 224 |
| 20 wt % PAF-1-NMDG | 220 |

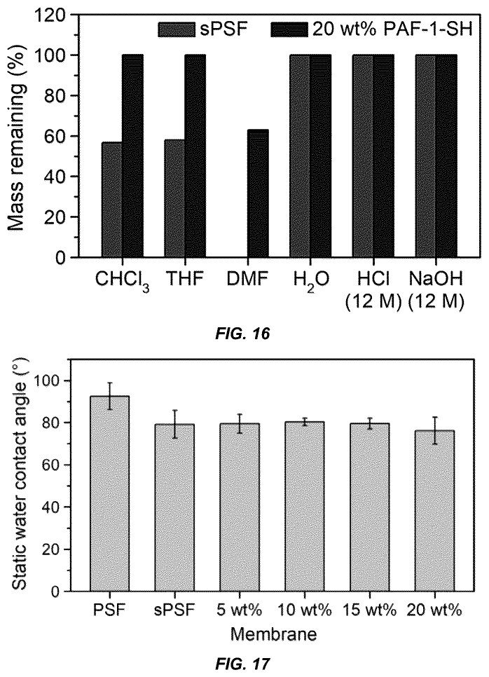

Membrane dissolution studies. Membrane dissolution studies were conducted to probe the abundance and strength of interfacial interactions between the PAFs and polymer matrix. Membrane samples (˜6 mg) consisting of neat sPSF or 20 wt % PAF-1-SH in sPSF were first transferred to 4-mL glass vials and dried for 48 h in a vacuum oven (100° C.) before they were quickly weighed on a microbalance. At room temperature, ˜4 mL of water, concentrated HCl (12 M), NaOH (12 M), or a solvent used frequently for membrane casting (CHCl₃, THF, DMF) were added to the vials. The solutions were occasionally shaken lightly. Each membrane sample was fully submerged in the solvents rather than resting on top of the solvent for the entirety of the tests. After 24 h of solvent immersion, the resulting solutions were removed from the vials and discarded along with any small pieces broken off of the remaining membranes. The vials were then dried for 48 h in a vacuum oven (100° C.) before quickly weighed on a microbalance. The masses remaining of the membrane samples in the vials after the solvent submersion are reported in FIG. 16.

Water uptake, swelling, and contact angle. Water uptake and swelling are regarded as two of the most important properties that affect ion transport in ion exchange membranes. Composite membranes with different PAF-1-SH loadings (0, 5, 10, 15, or 20 wt %) in sPSF were first converted to the H′ counterion form (i.e., sulfonate groups were ion exchanged with H⁺) for consistency and reproducibility purposes. Fabricated membranes were first submerged in a 1 M HCl solution for at least 24 h. This solution was replaced at least twice during the submersion period. The membranes were then submerged in DI water for at least 48 h to remove bulk HCl from the membranes. The DI water was replaced at least five times during the submersion period. After carefully blotting the membranes with a Kimwipe to remove excess water, the wet mass (m_{wet}) and wet length (l_{wet}) of each membrane were measured. The membranes were then dried in a vacuum oven for 48 h at 80° C. before the dry mass (m_{dry}) and dry length (l_{dry}) of each membrane were quickly measured. The water uptake (WU, %) and swelling ratio (SR, %) were calculated according to Eqs. S3 and S4, respectively:

$$WU = 100 \% \times ( \frac{{m}_{wet} - {m}_{dry}}{{m}_{dry}} ) \qquad ( S3 )$$

$$SR = 100 \% \times ( \frac{{l}_{wet} - {l}_{dry}}{{l}_{dry}} ) \qquad ( S4 )$$

Water uptake and swelling ratio values for the composite PAF-1-SH membranes are plotted in FIG. 2. Reported values and error bars represent the mean and standard deviation, respectively, obtained from measurements on at least five separately fabricated membranes at each PAF-1-SH loading.

The static water contact angle of each composite membrane (FIG. 17) was also measured to study the impact of the PAFs on membrane surface hydrophilicity. A contact angle goniometer (VCA Optima, AST Products, Inc.) was operated at ambient conditions. The contact angle was recorded ˜0.5 s after DI water (2 μL) was dropped onto the membrane surface for each measurement. Reported contact angle values and error bars represent the mean and standard deviation, respectively, obtained from measurements on five randomly selected locations on each sample.

Determination of PAF vol % loadings via pycnometry. The skeletal densities of sPSF and PAF-1-SH were measured using a helium pycnometer (Micromeritics AccuPyc II 1340) situated in a N₂-purged glove bag to prevent moisture effects. Prior to the measurements, sPSF and PAF-1-SH were ground into fine powders and dried overnight under vacuum at 60 and 110° C., respectively. In a N₂-purged glove tent, ˜1 g of the dried sPSF or ˜175 mg of the dried PAF-1-SH was transferred to a 3.5-mL pycnometer sample container and weighed. For each pycnometer measurement, 20 cycles were collected. Measurements collected in the final five cycles were used for density determination. The recorded skeletal densities of 1.337 g mL⁻¹ for sPSF and 1.368 g mL⁻¹ for PAF-1-SH represent the average data from four separate pycnometer measurements for each material.

To account for porosity, the bulk density of PAF-1-SH (0.420 g mL⁻¹) was determined based on the skeletal density and the amount of N₂ gas uptake at P/P₀=0.98 (47.4 mmol g⁻¹; see FIG. 10).

The measured PAF-1-SH wt % loadings in the composite membranes (determined by TGA decompositions; see Table B) were then converted to PAF-1-SH vol % loadings (Table B) using the measured bulk densities of sPSF and PAF-1-SH.

To confirm the accuracy of the pycnometer measurements, the density of polysulfone resin (M_{W}=60,000, Acros Organics) was also measured. A 3.5-mL sample container was loaded with 1.0 g of the dried resin. The measured polysulfone density (1.245 g mL⁻¹) closely aligns with the density reported by the manufacturer (1.240 g mL⁻¹).

Preparation of solutions simulating realistic water sources. To assess the performance of these materials and the versatility of an ion-capture electrodialysis (IC-ED) system in a variety of practical applications, four synthetic water solutions were prepared to mimic diverse water sources: low-salinity groundwater, brackish water, industrial wastewater, and seawater. The targeted and measured ion contents of these solutions are listed in Tables D and E. Groundwater (pH=7.1) was prepared according to ERMCA616 Groundwater certified reference material standards. Brackish water (pH=7.4) was prepared to mimic reported brackish water in the arid region of Phoenix, AZ. Industrial wastewater (pH=4) was prepared to contain the most common trace metal cations found in wastewater along with other common cations. Seawater (pH=8.2) was prepared according to ASTM D1411 Synthetic Seawater certified reference material standards. These solutions vary in pH, total competing ion content, and ion types, demonstrating a wide range of potential water solutions. To simplify adsorption and electrodialysis experimental conditions and analyses, all solutions were prepared using DI water (Milli-Q RiOs), as well as nitrate as the counterion to prevent formation of insoluble compounds in the presence of other anions (e.g., HgF₂, PbCl₂) or complex mercury anions (e.g., HgCl₄²⁻ at high Cl⁻ concentrations).

TABLE D
Ion contents of prepared solutions representing diverse practical water sources. Solutions
were prepared using metal nitrate salts. Expected concentrations are based on certified
reference material standards or other targeted concentrations, while measured concentrations
were quantified via ICP-OES. All quantities are reported in ppm.

| | Groundwater ^{a} | | Brackish water ^{b} | | Industrial | | Seawater ^{d} | |
|---|---|---|---|---|---|---|---|---|
| | Expected | Measured | Expected | Measured | Expected | Measured | Expected | Measured |
| Na⁺ | 27.9 | 27.5 | 849 | 837 | 100 | 99 | 11,031 | 11,007 |
| K⁺ | 5.8 | 5.7 | — | — | — | — | 398 | 395 |
| Mg²⁺ | 10.1 | 10.5 | 514 | 509 | 100 | 100 | 1,327 | 1306 |
| Ca²⁺ | 42.6 | 42.3 | 1,330 | 1,302 | 500 | 520 | 419 | 421 |
| Sr²⁺ | — | — | — | — | — | — | 13.8 | 13.4 |
| Ba²⁺ | — | — | 2.0 | 2.5 | — | — | — | — |
| Fe³⁺ | — | — | 2.3 | 2.2 | — | — | — | — |
| Other ^{c} | — | — | — | — | 35 | 34 | — | — |
| NO₃^{-e} | 268 | — | 9,037 | — | 2,393 | — | 38,469 | — |
| All | 86.4 | 86.0 | 2,697 | 2,653 | 735 | 753 | 13,189 | 13,142 |
| cations | | | | | | | | |
| Total | 354 | — | 11,734 | — | 3,128 | — | 51,658 | — |
| dissolved | | | | | | | | |
| solids ^{f} | | | | | | | | |

^{a} Groundwater (measured pH ≈ 7.0) was prepared to match cation concentrations in ERMCA616 Groundwater certified reference material standards.
^{b} Brackish water (measured pH ≈ 7.5) was prepared to match cation concentrations in reported brackish water sources in Phoenix, AZ, U.S (40).
^{c} Industrial wastewater (measured pH ≈ 4.0) was prepared to contain common cations (100 ppm each Na⁺ and Mg²⁺; 500 ppm Ca²⁺) and competing heavy metals (5 ppm each Mn²⁺, Fe³⁺, Ni²⁺, Cu²⁺, Zn²⁺, Cd²⁺, Pb²⁺) most common in wastewater sources (41). Other: see table S5.
^{d} Seawater (measured pH ≈ 8.0) was prepared to match cation concentrations in ASTM D1411 Synthetic Seawater certified reference material standards.
^{e} NO₃⁻ expected concentrations were calculated by assuming NO₃⁻ as the only anion present.
^{f} Theoretical total dissolved solids were calculated as the sum of the total cations and anions in each solution.

TABLE E
Concentrations of heavy metals in the synthetic industrial wastewater
solution. Solutions were prepared using metal nitrate salts.
Expected concentrations are based on targeted concentrations,
while measured concentrations were quantified via ICP-OES.

| Ion | Expected (ppm) | Measured (ppm) |
|---|---|---|
| Mn²⁺ | 5.0 | 4.9 |
| Fe³⁺ | 5.0 | 4.2 |
| Ni²⁺ | 5.0 | 4.5 |
| Cu²⁺ | 5.0 | 4.9 |
| Zn²⁺ | 5.0 | 5.4 |
| Cd²⁺ | 5.0 | 5.1 |
| Pb²⁺ | 5.0 | 4.8 |

Batch ion adsorption studies. All Hg²⁺ adsorption experiments and measurements were conducted in a dark environment to avoid the possible photoreduction of mercury. Ion concentrations were quantified via inductively coupled plasma optical emission spectrometry (ICP-OES, Optima 7000 DV Spectrometer). Samples containing mercury were prepared for ICP-OES measurements by diluting into a matrix of 5% HCl (TraceMetal Grade, Fisher Chemical) containing 5 ppm Au ions (Inorganic Ventures, Christiansburg, VA) in DI water. This matrix is known to prevent mercury memory effects that cause inaccurate ICP readings (42). A matrix of 5% HNO₃ (TraceMetal Grade, Fisher Chemical) in DI water was used for measuring all other ions. Samples were measured against calibration curves with known metal concentrations prepared from certified standards (Inorganic Ventures and Sigma-Aldrich), and extended wash times were applied to further prevent memory effects.

Ion adsorption capacities (Qₑ, mg g⁻¹ or mmol g⁻¹) were calculated using the equation:

$${Q}_{e} = \frac{( {C}_{0} - {C}_{e} ) V}{m} \qquad ( S5 )$$

where C₀ and Cₑ are the initial and equilibrium ion concentrations (mg L⁻¹), respectively, V is the solution volume (L), and m is the dry adsorbent mass (g).

For reproducibility purposes, membranes fabricated from bare sPSF or 20 wt % PAF-1-SH in sPSF were first converted to the Na⁺ counterion form prior to adsorption tests. Membranes were first submerged in a 1 M NaNO₃ solution for at least 24 h. This solution was replaced at least twice during the submersion period. The membranes were then submerged in DI water for at least 48 h to remove bulk NaNO₃ from the membranes. The DI water was replaced at least five times during this submersion period.

Control experiments were also performed to measure any He losses in solution caused by Hg²⁺ sticking to plastic. Each Hg²⁺ solution was shaken for 16 h in a plastic 4-mL or 20-mL vial (no PAF-1-SH or membrane sample was added) and filtered through a 0.45-μm polyethersulfone syringe filter (Nalgene). No measurable Hg²⁺ losses were identified in any of the solutions using these testing conditions.

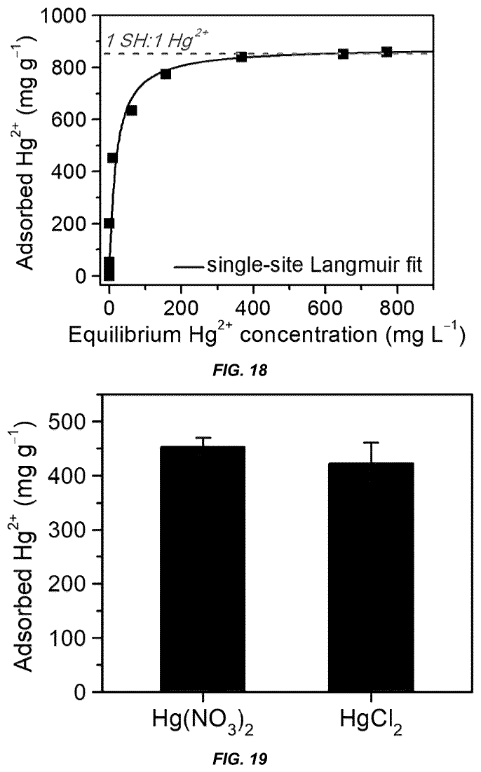

Equilibrium Hg²⁺ adsorption isotherm of PAF-1-SH powder. After drying, PAF-1-SH (0.8 mg) was quickly weighed in a plastic 4-mL vial using a microbalance rated and calibrated to 1 μg accuracy (Mettler MX5 Microbalance, Mettler Toledo). An aqueous Hg(NO₃)₂ solution (4 mL) in DI water within a range of Hg²⁺ concentrations (10 to 1,000 ppm) was then added to the vial, which was then sonicated until the PAF-1-SH was completely dispersed without agglomerations (˜1-5 min). The mixture was then shaken for 12 h at 300 rpm and 25° C. before filtered through a 0.45-μm polyethersulfone syringe filter (Nalgene) to remove the particles. The Hg²⁺ concentration of the filtered solution was measured via ICP-OES, and the amount of Hg²⁺ adsorbed in the material was calculated using Eq. S5. The experiment was repeated for various Hg²⁺ initial concentrations (FIG. 18). An analogous procedure using an aqueous HgCl₂ solution (100 ppm) was performed to confirm the high adsorption affinity of Hg²⁺ by PAF-1-SH in the presence of Cl⁻ counterions (FIG. 19).

Equilibrium Hg²⁺ adsorption isotherms of membranes. Membranes were converted to the Na⁺ counterion form prior to testing. After drying, sPSF (10 mg) and 20 wt % PAF-1-SH (10 mg) membrane pieces were quickly weighed and transferred to separate plastic 20-mL vials each containing a magnetic stir bar. An aqueous Hg(NO₃)₂ solution (20 mL) in DI water within a range of Hg²⁺ concentrations (10 to 550 ppm) was then added to each vial. The added solutions were stirred for 48 h at ˜500 rpm before the Hg²⁺ concentration in each solution was measured via ICP-OES. The amounts of Hg²⁺ adsorbed in each membrane was calculated using Eq. S5. The experiment was repeated for various Hg²⁺ initial concentrations (FIG. 2C). Expected 20 wt % Hg²⁺ uptake values reported in FIG. 2C correspond to the weighted average of the uptake determined from a Langmuir fit of the Hg²⁺ adsorption curves for the PAF-1-SH powder (FIG. 18, 20% contribution) and sPSF membrane (FIG. 2C, 80% contribution).

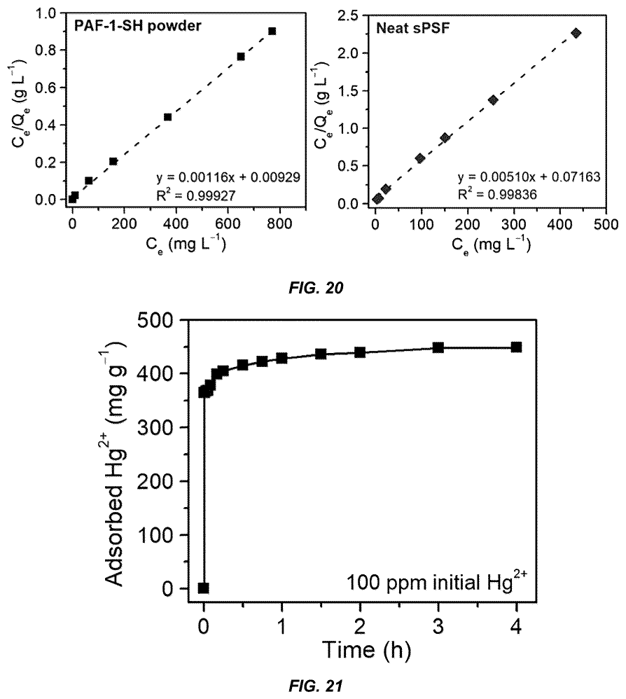

Modeling equilibrium Hg²⁺ uptake. The experimental Hg²⁺ equilibrium adsorption capacity values for the PAF-1-SH powder and sPSF membrane were fitted using the linearized form of the single-site Langmuir model, given by:

$$\frac{{C}_{e}}{{Q}_{e}} = \frac{{C}_{e}}{{Q}_{m}} + \frac{1}{{Q}_{m} {K}_{L}} \qquad ( S6 )$$

where Cₑ is the equilibrium Hg²⁺ concentration in the external solution (mg L⁻¹), Qₑ is the equilibrium Hg²⁺ adsorption capacity (mg g⁻¹) calculated from Eq. S5, Q m is the saturation Hg²⁺ adsorption capacity (mg g⁻¹), and K_{L} is the Langmuir constant (L mg⁻¹). Cₑ and Qₑ experimental values were plotted (FIG. 20) to extract Qₘ and K_{L} based on the slope and y-intercept values of these plots.

Since the composite membranes feature two chemically distinct binding modes (binding to PAF-1-SH, and ion exchange to the sPSF matrix), the dual-site Langmuir model was used to fit Hg²⁺ equilibrium adsorption capacity values for the 20 wt % PAF-1-SH in sPSF membranes. The dual-site Langmuir model is given by:

$${Q}_{e} = \frac{{C}_{e} {Q}_{m , 1} {K}_{L , 1}}{1 + {C}_{e} {K}_{L , 1}} + \frac{{C}_{e} {Q}_{m , 2} {K}_{L , 2}}{1 + {C}_{e} {K}_{L , 2}} \qquad ( S7 )$$

where Qₑ is the equilibrium Hg²⁺ adsorption capacity (mg g⁻¹) calculated from Eq. S5, Cₑ is the equilibrium Hg²⁺ concentration in the external solution (mg L⁻¹), Q_{m,1} and Q_{m,2} are the saturation Hg²⁺ adsorption capacities (mg g⁻¹) of the PAF-1-SH and sPSF adsorption sites, respectively, and K_{L,1} and K_{L,2} are the Langmuir constants (L mg⁻¹) of the PAF-1-SH and sPSF sites, respectively. Nonlinear regression was used to fit the dual-site Langmuir model.

Fitted Langmuir model parameters, along with additional details for determining the percentage of PAF-1-SH binding sites that remain accessible within the membrane matrix, are provided in Table F.

TABLE F
Langmuir model fitting parameters for the collected Hg²⁺
equilibrium adsorption isotherms (see FIG. 18 and Fig. 2C). Q_{m, 1} and
Q_{m, 2} are the saturation Hg²⁺ adsorption capacities of two distinct
adsorption sites, and K_{L, 1} and K_{L, 2} are the Langmuir constants
of the two adsorption sites.

| | Q_{m, 1} | K_{L, 1} | Q_{m, 2} | K_{L, 2} |
|---|---|---|---|---|
| Material | (mg g⁻¹) | (L mg⁻¹) | (mg g⁻¹) | (L mg⁻¹) |
| PAF-1-SH ^{a} | 862 | 0.125 | — | — |
| Neat sPSF ^{a} | 196 | 0.071 | — | — |
| 20 wt % PAF-1-SH ^{b} | 161 | 0.114 | 157 | 0.039 |

^{a} A single-site Langmuir model was used to fit the Hg²⁺ adsorption isotherms of the PAF-1-SH powder and neat sPSF membrane. Here, Q_{m, 1} and K_{L, 1} are equivalent to Qₘ and K_{L}, respectively, in Eq. S5. The adsorption site for sPSF results from simple ion exchange, which exhibits relatively low ion selectivity (FIG. 2D) and does not lead to appreciable ion capture in an IC-ED process (table S7). Nonetheless, sPSF adsorption was included for accuracy in modeling PAF-1-SH adsorption accessibility in the composite membranes.
^{b} A dual-site Langmuir model was used to fit the Hg²⁺ adsorption isotherm of the 20 wt % PAF-1-SH in sPSF membrane. Q_{m, 1} and K_{L, 1} values correspond to the PAF-1-SH adsorption site, while Q_{m, 2} and K_{L, 2} values correspond to the sPSF adsorption site. Nonlinear regression was used to fit the data. The Q_{m, 2} value was set to 80% of the Qₘ value for neat sPSF (157 mg g⁻¹; i.e., all sPSF sites were assumed to remain accessible in the 20 wt % PAF-1-SH membrane). Q_{m, 1} was constrained to have a maximum value corresponding to 20% of the Q_{m, 1} value for PAF-1-SH powder (172.4 mg g⁻¹). K_{L, 1} and K_{L, 2} were constrained to have maximum values corresponding to the K_{L, 1} value for PAF-1-SH powder and neat sPSF, respectively. Based on the Q_{m, 1} experimental value (161 mg g⁻¹) compared to the theoretical maximum value (172.4 mg g⁻¹, or 20% of the Q_{m, 1} value for PAF-1-SH powder), the percentage of PAF-1-SH adsorbent sites that remain accessible within the membrane matrix was determined to be 93%.

Hg²⁺ adsorption kinetics of PAF-1-SH powder. After drying, PAF-1-SH (4 mg) was quickly weighed using a microbalance and transferred to a plastic 20-mL vial containing a magnetic stir bar. DI water (18.67 mL) was then added to the vial, and the mixture was sonicated until the PAF-1-SH was completely dispersed without agglomerations (˜10 min). While stirring at ˜1,000 rpm at ambient conditions, an aqueous Hg(NO₃)₂ solution (1.33 mL, 1,500 ppm Hg²⁺ in DI water) was then added to the vial to reach the final desired Hg²⁺ concentration (100 ppm). The solution was continuously stirred at ˜1,000 rpm while 750-μL aliquots of the solution were collected at fixed time intervals. These aliquots were immediately filtered through a 0.45-μm polyethersulfone syringe filter, and the Hg²⁺ concentrations in the filtered solutions were measured via ICP-OES. The amount of Hg²⁺ adsorbed in the material at each time interval (FIG. 21) was calculated using Eq. S5.

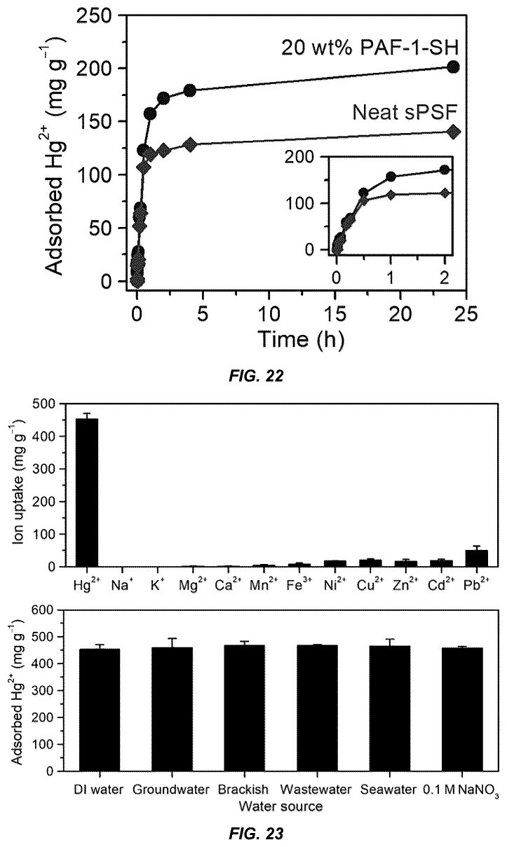

Hg²⁺ adsorption kinetics of membranes. Membranes were converted to the Na⁺ counterion form prior to testing. After drying, sPSF (10 mg) and 20 wt % PAF-1-SH in sPSF (10 mg) membranes were quickly cut into several small pieces and weighed before transferred to separate plastic 20-mL vials each containing a magnetic stir bar. DI water (18 mL) was then added to each vial, and the solution was lightly stirred overnight (˜200 rpm) to ensure water uptake and swelling of the membranes approximately reached equilibrium states prior to testing. While stirring at ˜1,000 rpm at ambient conditions, an aqueous Hg(NO₃)₂ solution (2 mL, 1,500 ppm Hg²⁺ in DI water) was then added to each vial to reach the final desired Hg²⁺ concentration (150 ppm). The solutions were kept stirring at ˜900 rpm while 200-μL aliquots of the solutions were collected at fixed time intervals. The Hg²⁺ concentrations in these aliquots were measured via ICP-OES. The amounts of Hg²⁺ adsorbed in the membrane materials at each time interval (FIG. 22) were calculated using Eq. S5.

Ion adsorption selectivity of PAF-1-SH powder. To investigate the binding affinity of PAF-1-SH powder for Hg²⁺ over other common competing ions in water, single ion adsorption experiments were performed. After drying, PAF-1-SH (0.8 mg) was quickly weighed in a plastic 4-mL vial using a microbalance. An aqueous solution (4 mL) containing 0.5 mM of one type of ion (Na⁺, K⁺, Mg²⁺, Mn²⁺, Fe²⁺, Ni²⁺, Cu²⁺, Zn²⁺, Cd²⁺, or Hg²⁺) with NO₃⁻ as the counterion in DI water was then added to the vial. The mixture was then sonicated until the PAF-1-SH was completely dispersed without visible agglomerations (˜1-5 min). The mixture was then shaken for 16 h at 300 rpm and 25° C. before it was filtered through a 0.45-μm polyethersulfone syringe filter to remove the particles. The ion concentration of the filtered solution was measured via ICP-OES, and the amount of the ion adsorbed in the material (FIG. 23) was calculated using Eq. S5. The experiment was repeated for each type of ion listed. In the Fe³⁺ solution, citric acid (1 equiv) was also added to lower the pH to ˜3 to prevent Fe(OH)₃ precipitation. Reported values and error bars represent the mean and standard deviation, respectively, obtained from measurements on at least three different samples.

Hg²⁺ adsorption selectivity in realistic water sources. To probe the multicomponent binding selectivity of PAF-1-SH powder for Hg²⁺, adsorption experiments were conducted using Hg²⁺ spiked in a wide variety of practical, complex aqueous solutions (synthetic groundwater, synthetic brackish water, synthetic industrial wastewater, and synthetic seawater). After drying, PAF-1-SH (0.8 mg) was quickly weighed in a plastic 4-mL vial using a microbalance. An aqueous solution (4 mL) containing Hg²⁺ (100 ppm, or ˜0.5 mM) in one of the realistic water sources was then added to the vial. Afterward, the mixture was sonicated until the PAF-1-SH was completely dispersed without visible agglomerations (˜1 to 5 min). The mixture was then shaken for 16 h at 300 rpm and 25° C. before being filtered through a 0.45-μm polyethersulfone syringe filter (Nalgene) to remove the particles. The Hg²⁺ concentration of the filtered solution was measured via ICP-OES, and the amount of Hg²⁺ adsorbed in the material (FIG. 23) was calculated using Eq. S5. The experiment was repeated for each aqueous solution listed. Reported values and error bars represent the mean and standard deviation, respectively, obtained from measurements on at least three different samples.

Analogous adsorption experiments were conducted using neat sPSF or membranes consisting of 20 wt % PAF-1-SH in sPSF to examine whether the PAF particles maintain high ion selectivity within a composite matrix. Membranes were converted to the Na⁺ counterion form prior to testing. After drying, sPSF (10 mg) and 20 wt % PAF-1-SH (10 mg) membrane pieces were quickly weighed and transferred to separate plastic 20-mL vials each containing a magnetic stir bar. An aqueous solution (20 mL) containing Hg²⁺ (100 ppm, or ˜0.5 mM) in one of the realistic water sources was then added to each vial. The added solutions were stirred for 48 h at ˜500 rpm before the Hg²⁺ concentrations in the solutions were measured via ICP-OES. The amounts of Hg²⁺ adsorbed in the membrane materials (FIG. 2D) were calculated using Eq. S5. The experiment was repeated for each type of practical aqueous solution listed. Reported values and error bars represent the mean and standard deviation, respectively, obtained from measurements on at least three different samples. Expected 20 wt % Hg²⁺ uptake values reported in FIG. 2D correspond to the weighted average of the Hg²⁺ capacities measured for the PAF-1-SH powder (FIG. 23) and sPSF membrane (FIG. 2D).

Recovery of adsorbed target ion and reusability of composite membranes. To recover the adsorbed Hg²⁺ and determine the reusability of the membranes for selective ion capture, adsorption-desorption experiments were performed over ten cycles. For adsorption, a piece of a dried 20 wt % PAF-1-SH in sPSF membrane (10 mg) was quickly weighed and transferred to a plastic 20-mL vial containing a magnetic stir bar. An aqueous Hg(NO₃)₂ solution in DI water (20 mL, 100 ppm Hg²⁺) was then added to the vial. The added solution was stirred for 48 h at ˜500 rpm before the Hg²⁺ concentration in the solution was measured via ICP-OES. The amount of Hg²⁺ adsorbed in the membrane (FIG. 2E) was calculated using Eq. S5.

For desorption, the membrane was then regenerated using a series of HCl and NaNO₃ washes. Concentrated HCl is known to effectively regenerate the thiol in porous adsorbents while forming a stable mercury anionic species predominant at chloride concentrations above 1 M:

RS—Hg⁺+4HCl↔RS—H+HgCl₄²⁻+3H⁺ (S8)

Here, R is the PAF backbone to which the thiol is appended. The membrane was sonicated in concentrated HCl (20 mL, 12.1 M) for 1.5 h before then being sonicated for 1.5 h in a solution of NaNO₃ in DI water (2 M, 20 mL). The NaNO₃ solution was used to replace Hg²⁺ ion exchanged with the sPSF matrix upon desorption from PAF-1-SH. This HCl and NaNO₃ washing procedure was repeated three times. The Hg²⁺ concentration in each washing solution was measured via ICP-OES to confirm the successful recovery of the targeted Hg²⁺ ion. The total desorbed Hg²⁺ amount is reported in FIG. 2E as the combined mg of Hg²⁺ recovered in these washing solutions per dry g of the membrane. Before performing the next adsorption cycle, the membrane was submerged in DI water for at least 48 h to remove bulk NaNO₃ from the membrane. This DI water bath was replaced at least five times during the submersion period. These adsorption and desorption experiments were repeated nine times for a total of ten cycles.

Preliminary optimization of regeneration conditions. With the goal of reducing the resource intensity needed to achieve target ion recovery and membrane regeneration, we carried out additional adsorption-desorption experiments using only HCl for regeneration. For adsorption, a piece of a dried 20 wt % PAF-1-SH in sPSF membrane (10 mg) was quickly weighed and transferred to a plastic 20-mL vial containing a magnetic stir bar. A solution of Hg(NO₃)₂ in DI water (20 mL, 100 ppm Hg²⁺) was then added to the vial. The added solution was stirred for 72 h at ˜500 rpm before the Hg²⁺ concentration was measured via ICP-OES. The amount of Hg²⁺ adsorbed in the membrane (˜180 mg g⁻¹) was calculated using Eq. S5. The adsorption experiment was repeated four times using new membrane samples to obtain five separate He-adsorbed samples.

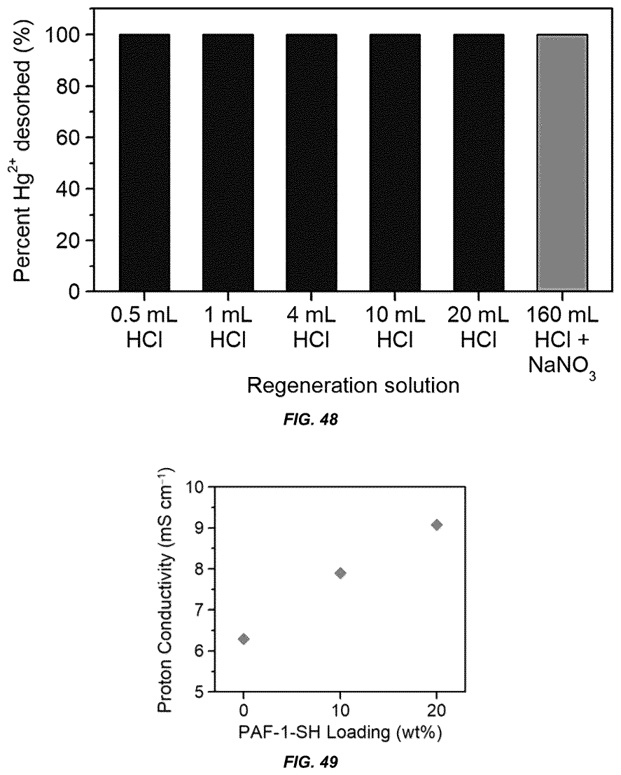

Each membrane sample was then regenerated using one of five volumes of concentrated (12.1 M) HCl: 0.5, 1, 4, 10, or 20 mL. Each membrane sample was retrieved from the adsorption solution, wiped, and cut into several small pieces before being transferred into a 0.65-mL or 1.5-mL plastic microcentrifuge tube (for the 0.5 or 1-mL HCl samples, respectively), a 4-mL glass vial (for the 4-mL HCl sample), or a 20-mL glass vial (for the 10 and 20-mL HCl samples). Each container was equipped with a small magnetic stir bar. The aforementioned volumes of concentrated HCl were then added to each corresponding sample. The added solutions were stirred for 72 h at ˜500 rpm before the Hg²⁺ concentration in each solution was measured via ICP-OES. The mg of desorbed Hg²⁺ per g of dry membrane was calculated using Eq. S5. The percentage of Hg²⁺ desorbed by each solution volume (FIG. 48) was calculated as the ratio of the desorbed Hg²⁺ amount to the adsorbed Hg²⁺ amount.

Equilibrium adsorption of target solutes by other selective PAFs. The adsorption capacities of other reported PAFs for their respective target solutes were measured and compared to capacity values reported in literature. The copper-selective PAF-1-SMe (0.8 mg) was dried and then quickly weighed in a plastic 4-mL vial using a microbalance. An aqueous Cu(NO₃)₂ solution (4 mL, ˜2 mM Cu²⁺) in 0.1 M HEPES buffer (Fisher Scientific, pH=6.5) was then added to the vial, which was then sonicated until the PAF was completely dispersed without agglomerations (˜5 min). The HEPES buffer was used to prevent copper precipitation and to match conditions reported in literature for proper comparison. The mixture was then shaken for ˜16 h at 300 rpm and 25° C. before being filtered through a 0.45-μm polyethersulfone syringe filter to remove the particles. The Cu²⁺ concentration of the filtered solution was measured via ICP-OES, and the amount of Cu²⁺ adsorbed in the material was calculated using Eq. S5. This procedure was repeated for the iron-selective PAF-1-ET (0.8 mg) using an aqueous Fe(NO₃)₃ solution (4 mL, ˜2 mM Fe³⁺, pH≈3 adjusted using ˜2 mM citric acid) in 0.1 M HEPES buffer, as well as for the boric acid-selective PAF-1-NMDG (0.8 mg) using an aqueous B(OH)₃ solution (4 mL, ˜2 mM boric acid) in DI water.

Design of electrochemical cells. Glass electrodialysis cells were custom-made at the College of Chemistry Glass Shop at the University of California, Berkeley. Three distinct sets of cells were constructed with different half-cell volumes of 45, 7.5, and 1.7 mL. The cells consisted of an NW16 glass flange connected to one of the following: a small glass tube (5 mm inner diameter) for the 1.7-mL half-cells, a GL-18 glass screw thread for the 7.5-mL half-cells, or a GL-45 glass screw thread for the 45-mL half-cells. GL-14 glass screw threads were also attached to the 7.5-mL and 45-mL half-cells; electrodes were inserted into these threads and kept in place using O-rings and Parafilm wrap. Borosilicate glass was used for all cell fabrication. Membranes were sandwiched between the flanges of two separate half-cells, which were fastened together using an 0-ring and knuckle clamp set.

A three-compartment cell was also custom-made to test the effectiveness of ion-capture electrodialysis in a working electrodialysis stack device. The 7.5-mL feed (middle) compartment consisted of a small glass tube (8 mm inner diameter) connected to two NW16 glass flanges. The 7.5-mL cell compartments used in the two-compartment electrodialysis experiments were used in the stack device as the cation receiving and anion receiving (side) compartments.

Ion-capture electrodialysis proof-of-concept experiments.

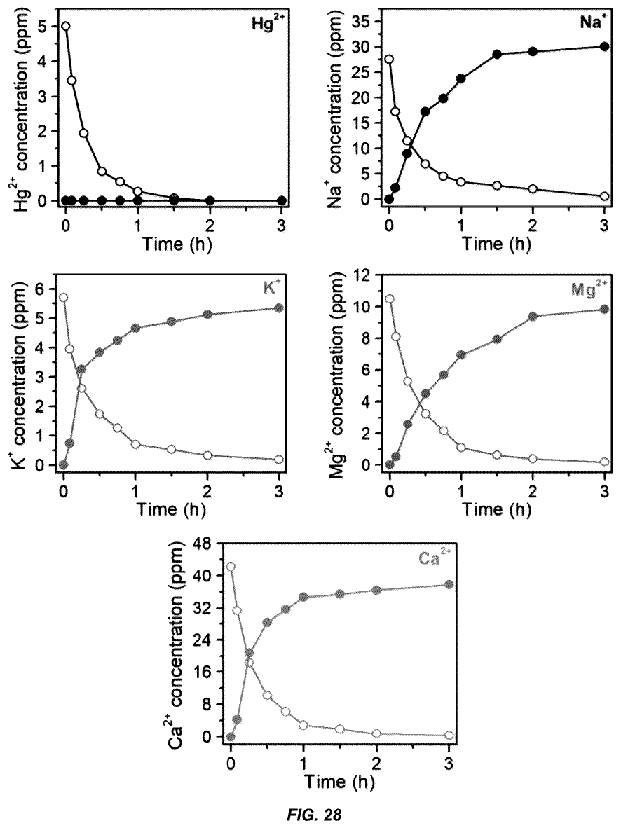

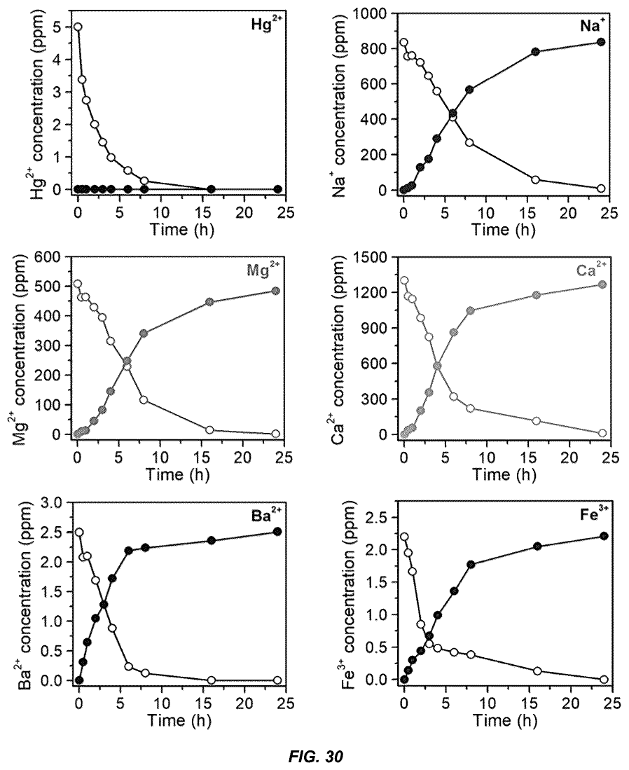

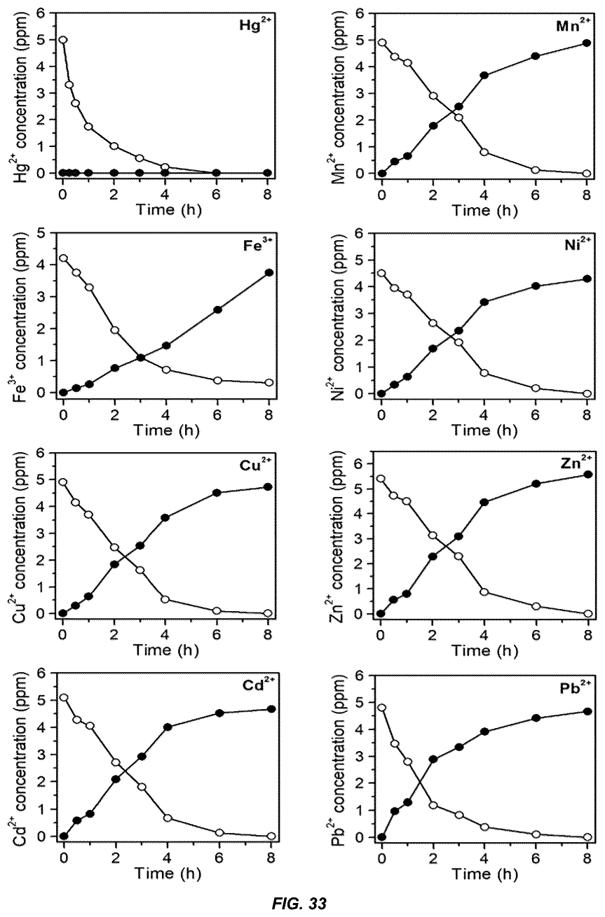

General experimental setup. All electrodialysis experiments and measurements were conducted in a dark environment to avoid the possible photoreduction of heavy metals. Prior to testing, all membranes were converted to the Li′ counterion form. Lithium ion was chosen as the initialized counterion because it is not present in any of the water source solutions treated in this study (Tables D and E), and thus any possible Li′ ion release into the feed or receiving half-cells during electrodialysis would not interfere with the reported cation concentrations (FIGS. 28, 30, 32, and 33). For this reason, Li′ ions potentially exchanged out of the membranes into the solutions during testing were also not measured or included in the reported ion concentration measurements. Membranes were first submerged in a 1 M LiNO₃ solution for at least 24 h. This solution was replaced at least twice during the submersion period. The membranes were then submerged in DI water for at least 48 h to remove bulk LiNO₃ from the membranes. The DI water was replaced at least five times during this submersion period.

In each test, a hydrated membrane (2.0 cm² active area) was clamped between two 7.5-mL half-cells. At room temperature, the solutions in both half-cells were constantly stirred at ˜1,000 rpm to diminish concentration polarization effects and homogenize the solutions for sampling. A platinum counter electrode (anode, Bioanalytical Systems, Inc., West Lafayette, IN, USA) was placed in the feed half-cell, and a glassy carbon working electrode (cathode, Bioanalytical Systems, Inc.) was inserted into the receiving half-cell. The “feed” half-cell (also known as the diluate) refers to the compartment initially containing the target ion, while the “receiving” half-cell (also known as the concentrate) refers to the other compartment. The electrodes were placed directly next to the membrane as close as possible to each other without touching the membrane. A Ag/AgCl reference electrode (3 M NaCl internal electrolyte, Bioanalytical Systems, Inc.) was inserted into the receiving half-cell as close as possible to the working electrode without touching the latter. The reference electrode was otherwise stored in a 3 M NaCl solution when not in use. To enable ion migration from the feed half-cell to the receiving half-cell, voltages were applied using a BioLogic SP-200 or SP-300 potentiostat and EC-Lab software. To account for any electrodeposited metals, the cathode was sonicated in concentrated HNO₃ (TraceMetal Grade) for ˜30 s each time an aliquot was collected from the receiving solution, and dissolved metals in this HNO₃ rinsing solution were measured. No deposited precipitates were visibly observed on the cathode after each HNO₃ wash, suggesting that all electrodeposited metals were sufficiently collected. The cathode was then quickly rinsed with DI water and wiped to remove residual moisture before reinserted into the receiving half-cell. Reported receiving half-cell concentrations represent the combined concentrations of this rinsing solution and the aliquot sample. All reported ion concentrations were measured using ICP-OES. In every experiment, both half-cells were capped loosely with a rubber septum and vented to ambient air to remove H₂ and O₂ formed at the cathode and anode, respectively. No solution leakages in the cells were detected in any of the reported experiments for the entirety of the tests.

The percentage of the target species captured from the feed solution was calculated using Eq. S9:

$$Target species captured ( \% ) = 100 \% \times ( \frac{{C}_{f , feed} + {C}_{f , receiving}}{{C}_{0 , feed} + {C}_{0 , receiving}} ) \qquad ( S9 )$$

where C_{f,feed} and C_{f,receiving} are the concentrations of the target species in the feed and receiving solutions, respectively, at the final time interval, and C_{0,feed} and C_{0,receiving} are the initial concentrations of the target species in the feed and receiving solutions, respectively, at time zero. No target species was added to or measured in any of the initial receiving solutions, but C_{0,receiving} is included in Eq. S9 for completeness. In the cases where no target species was measured in the final feed or receiving solutions, C_{f,feed} or C_{f,receiving} were taken to be the concentration detection limits of the used ICP-OES instrument when calculating the percentage of target species captured.

The percent feed desalination (i.e., deionization, or the percentage of all ions removed from the feed) was calculated using Eq. S10:

$$Desalination ( \% ) = 100 \% \times ( \frac{{C}_{f , feed , total}}{{C}_{0 , feed , total}} ) \qquad ( S10 )$$

where C_{f,feed,total} and C_{0,feed,total} are the sum of all measured cation concentrations in the feed solution at the final and initial time points, respectively. Anion concentrations were not measured and that desalination calculations were only based on cation concentrations, as proof-of-concept studies focused on selective cation transport. Analogous calculations can be performed for evaluating the separation performance of anion-capture electrodialysis membranes. Nonetheless, in a typical electrodialysis process, the amount of cationic charges that transport from the feed across the cation exchange membrane is expected to be approximately equal to the amount of anionic charges that transport from the feed across the analogous anion exchange membrane, to maintain electroneutrality. Thus, desalination calculations based on only cation concentrations are assumed to approximately reflect desalination calculations based on both cation and anion concentrations in an electrodialysis stack.

The IC-ED performances of all materials studied in this work, including their target species capture and desalination percentages, are compiled in Table G.

TABLE G
Summary of the proof-of-concept two-compartment ion-capture electrodialysis
and solute-capture diffusion dialysis performances by the various materials
in this work. NM = not measured; ND = not detected by ICP-OES.

| | | | Final feed | Final receiving | Target |
|---|---|---|---|---|---|
| | | | target species | target species | species |
| Membrane | Water source, | Desalination | concentration | concentration | captured |
| material | target species | (%) | (ppm) | (ppm) | (%) ^{a} |
| Neat sPSF | Groundwater, | NM | ND | 5.02 | 0 |
| | Hg²⁺ | | | | |
| 20 wt % | Groundwater, | 98.5 | ND | ND | >99 |
| PAF-1-SH | Hg²⁺ | | | | |
| Neat sPSF | Brackish water, | NM | 0.12 | 4.70 | 3.7 |
| | Hg²⁺ | | | | |
| 20 wt % | Brackish water, | 99.1 | ND | ND | >99 |
| PAF-1-SH | Hg²⁺ | | | | |
| Neat sPSF | Industrial | NM | 0.55 | 3.96 | 3.0 |
| | wastewater, | | | | |
| | Hg²⁺ | | | | |
| 20 wt % | Industrial | 97.5 | ND | ND | >99 |
| PAF-1-SH | wastewater, | | | | |
| | Hg²⁺ | | | | |
| Neat sPSF | 0.1M HEPES, | NM | 0.01 | 4.67 | 8.1 |
| | Cu²⁺ | | | | |
| 20 wt % | 0.1M HEPES, | 99.2 | ND | ND | >99.9 |
| PAF-1-SMe | Cu²⁺ | | | | |
| Neat sPSF | 0.1M HEPES, | NM | 0.25 | 1.89 | 6.6 |
| | Fe²⁺ | | | | |
| 20 wt % | 0.1M HEPES, | 96.0 | ND | ND | >99.5 |
| PAF-1-ET | Fe²⁺ | | | | |
| Neat sPSF | Groundwater, | NM | 2.27 | 2.19 | 0.9 |
| | B(OH)₃ | | | | |
| 20 wt % | Groundwater, | NM | ND | ND | >99.5 |
| PAF-1-NMDG | B(OH)₃ | | | | |

^{a} In cases where the target species were not detected in the final feed or receiving half-cell solutions, the final target species concentration was taken as the ICP-OES detection limit when
calculating the percentage of the target species captured.

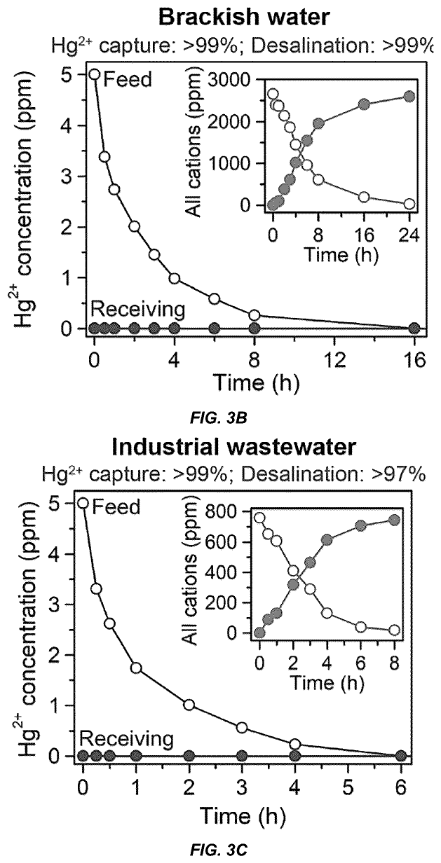

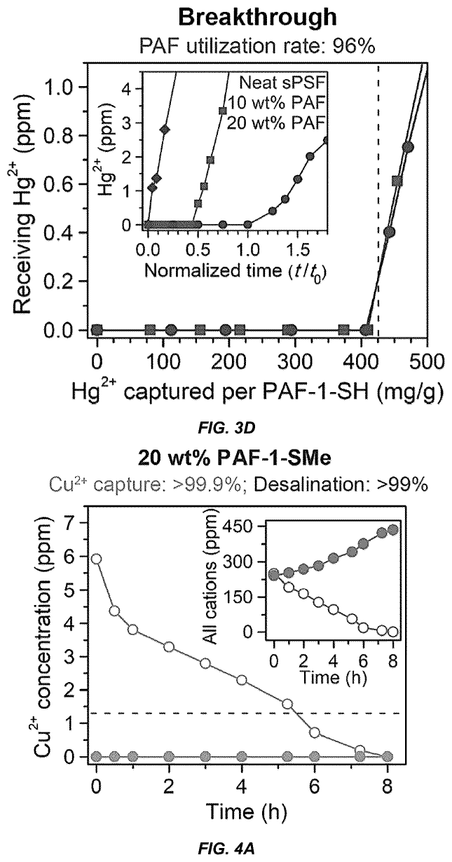

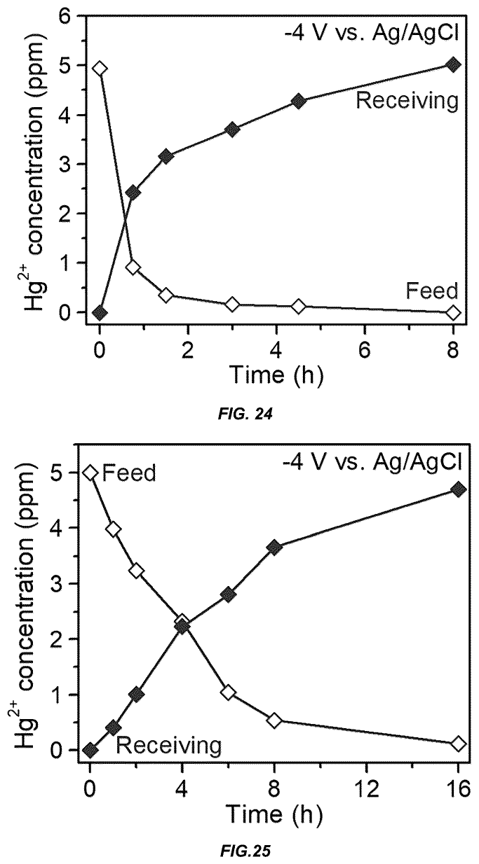

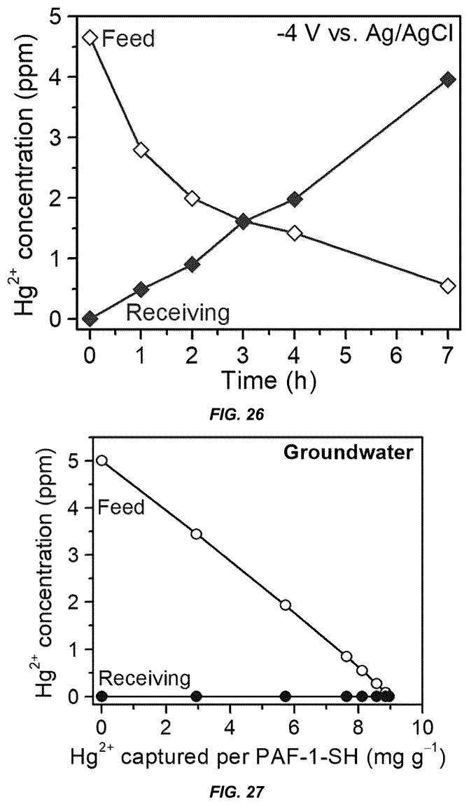

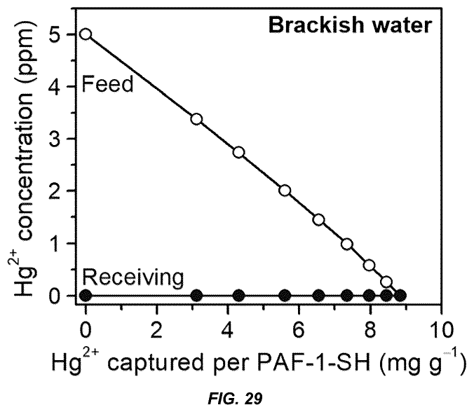

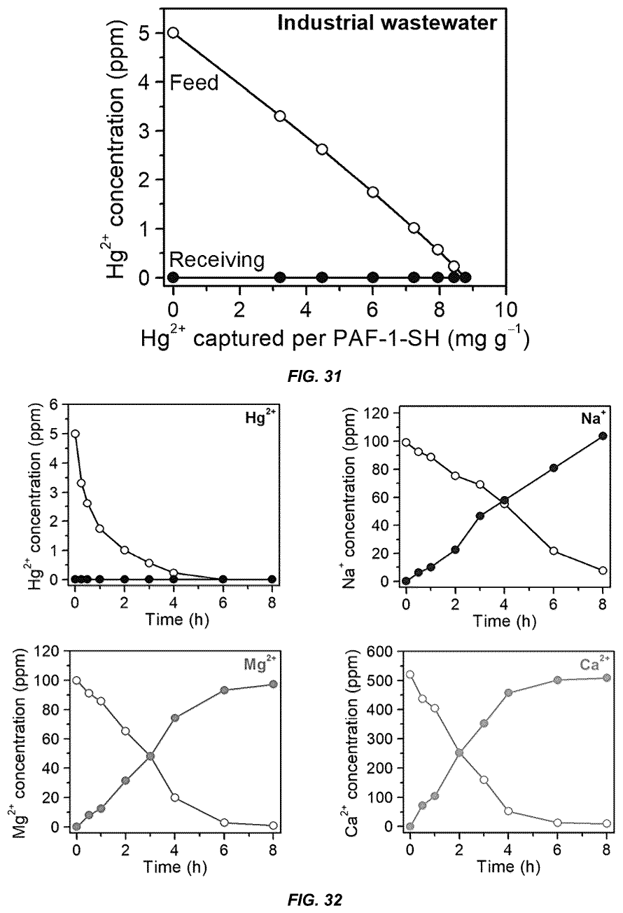

Hg²⁺-capture electrodialysis of various realistic water sources. 20 wt % PAF-1-SH in sPSF membranes were tested for He-capture electrodialysis using aqueous matrices mimicking three practical water sources (groundwater, brackish water, and industrial wastewater). The results of these tests are given in FIG. 3A-C. While stirring, 7.5 mL DI water containing 10 mM TraceMetal Grade HNO₃ (to maintain electrical conductivity and neutralize hydroxide formed at the cathode) was added to the receiving half-cell. An aqueous solution (7.5 mL) containing Hg(NO₃)₂ (5 ppm Hg²⁺) spiked in one of the practical water solutions was then added to the feed half-cell. The solutions were stirred for ˜10 s before aliquots were removed from each half-cell; the concentrations in these samples corresponded to t=0. A voltage of −4 V vs. Ag/AgCl was then immediately applied. For tests on groundwater or brackish water, 0.3-mL aliquots of the solutions in each half-cell were collected at fixed time intervals throughout the duration of the tests. For tests on industrial wastewater, a 0.15-mL aliquot for Hg²⁺ analysis and a separate 0.2-mL aliquot for the analysis of all other competing cations were removed from each half-cell at each time interval. In each test, solutions of HNO₃ (3 M) or LiOH (1 M) in DI water were periodically added to the receiving and feed half-cells, respectively, to maintain a pH between 2-8 in both half-cells as water splitting occurred. Changes to the concentration of each measured ion resulting from these dilutions were corrected in reported values according to the volumes of the added HNO₃ or LiOH solutions. Individual concentration profiles of Hg²⁺ and all relevant competing cations in each test are provided in FIGS. 28, 30, 32, and 33. Hg²⁺ concentration profiles plotted versus Hg²⁺-capture capacity are provided in FIGS. 27, 29, and 31 for context. For comparison, each experiment was repeated using a neat sPSF membrane (FIGS. 24 to 26).

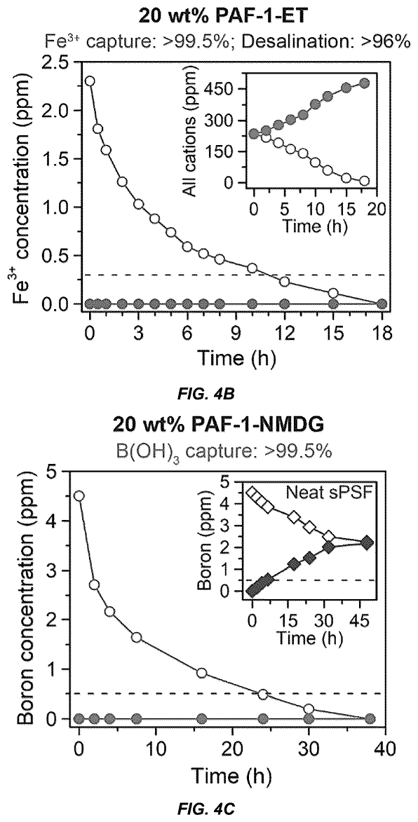

Cu²⁺-capture electrodialysis using copper-selective membranes. 20 wt % PAF-1-SMe in sPSF membranes were tested for C²⁺-capture electrodialysis (FIG. 4A). HEPES buffer (0.1 M, pH=6.5) was chosen as the aqueous matrix in both half-cells to supply relevant competing cations (measured as ˜240 ppm Na⁺) and prevent the precipitation of Cu(OH)₂ that occurs under alkaline conditions. While stirring, 3.75 mL HEPES buffer (0.2 M, pH=6.5), 0.075 mL HNO₃ (0.1 M, to reach the desired half-cell concentration of 1 mM), and 3.675 mL DI water were added to the receiving half-cell. 3.75 mL HEPES buffer (0.2 M, pH=6.5), 3.729 mL DI water, and 0.0206 mL of a Cu(NO₃)₂ solution (˜2,000 ppm in DI water) were added to the feed half-cell to reach the desired initial Cu²⁺ concentration of ˜6 ppm. A voltage of −2 V vs. Ag/AgCl was then applied. Aliquots of the solutions (0.225 mL) in each half-cell were collected and analyzed at fixed time intervals. Reported values and error bars represent the mean and range, respectively, obtained from measurements on two different samples. The pH in each half-cell was measured as 6.5 throughout the entirety of the experiments. For comparison, the experiments were repeated using a neat sPSF membrane (FIG. 37).

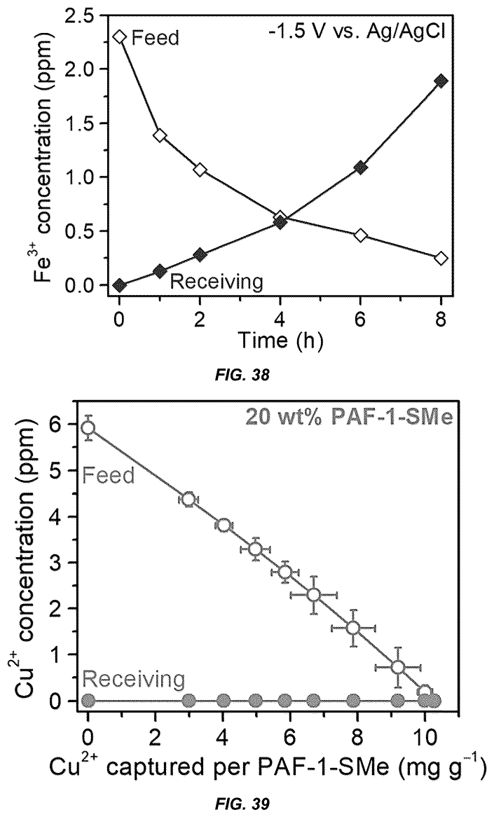

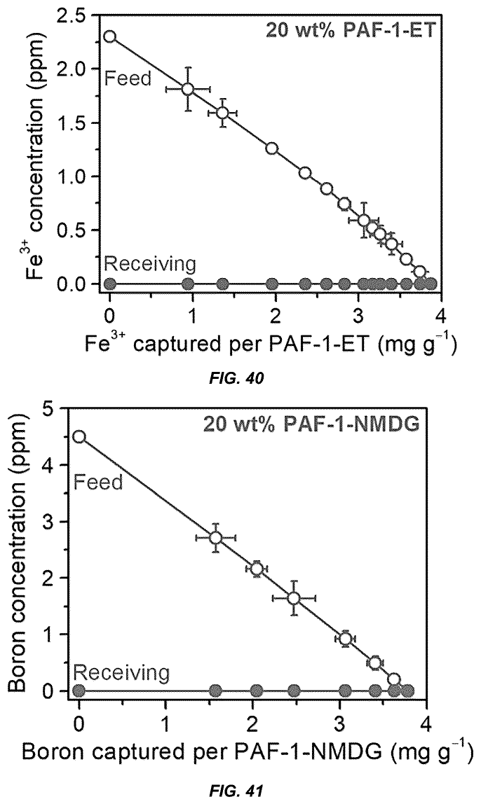

Fe³⁺-capture electrodialysis using iron-selective membranes. 20 wt % PAF-1-ET in sPSF membranes were tested for Fe³⁺-capture electrodialysis (FIG. 4B). HEPES buffer (0.1 M), which features a pKₐ₁≈3 buffer site, was chosen as the aqueous matrix in both compartments to prevent the precipitation of Fe(OH)₃ at higher pH values. While stirring, 3.75 mL HEPES buffer (0.2 M, pH=6.5) and 3.75 mL HNO₃ (0.1 M, to reach the desired half-cell concentration of 50 mM) were added to the receiving half-cell. 3.75 mL HEPES buffer (0.2 M, pH=6.5), 3.664 mL HNO₃ (0.1 M), and 0.0863 mL of an Fe(NO₃)₃ solution (˜200 ppm in DI water with pH=3 adjusted using 1 equiv citric acid) were added to the feed half-cell to reach the desired initial Fe³⁺ concentration of ˜2.3 ppm (mimicking typical iron concentrations in brackish water in Maricopa County, AZ). A voltage of −1.5 V vs. Ag/AgCl was then applied. Aliquots of the solutions (0.225 mL) in each half-cell were collected and analyzed at fixed time intervals. Reported values and error bars represent the mean and range, respectively, obtained from measurements on two different samples. The pH in each half-cell was measured to be between 2 to 4 throughout the entirety of the experiments. For comparison, the experiments were repeated using a neat sPSF membrane (FIG. 38).

Stack device utilizing ion-capture electrodialysis. Electrodialysis experiments using a home-built stack electrodialysis device were conducted. A three-compartment cell consisting of feed, cation receiving, and anion receiving compartments was employed. A hydrated cation exchange membrane consisting of neat sPSF or 20 wt % PAF-1-SH in sPSF was placed between the feed and cation receiving compartments. A hydrated Fumasep FAS-50 anion exchange membrane (Fuel Cell Store) was placed between the feed and anion receiving compartments. Prior to testing, the cation and anion exchange membranes were converted to the Li⁺ and NO₃⁻ counterion forms, respectively, using 1 M LiNO₃ and DI water submersion procedures. A platinum anode (Bioanalytical Systems, Inc.) was placed in the anion receiving compartment, and a glassy carbon cathode (Bioanalytical Systems, Inc.) was inserted into the cation receiving compartment. The electrodes were placed next to the membranes in their respective compartments but did not come into contact with the membranes.

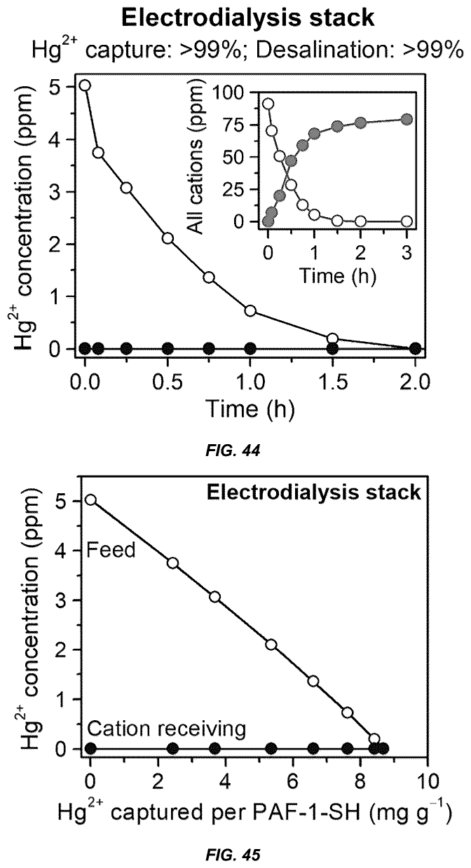

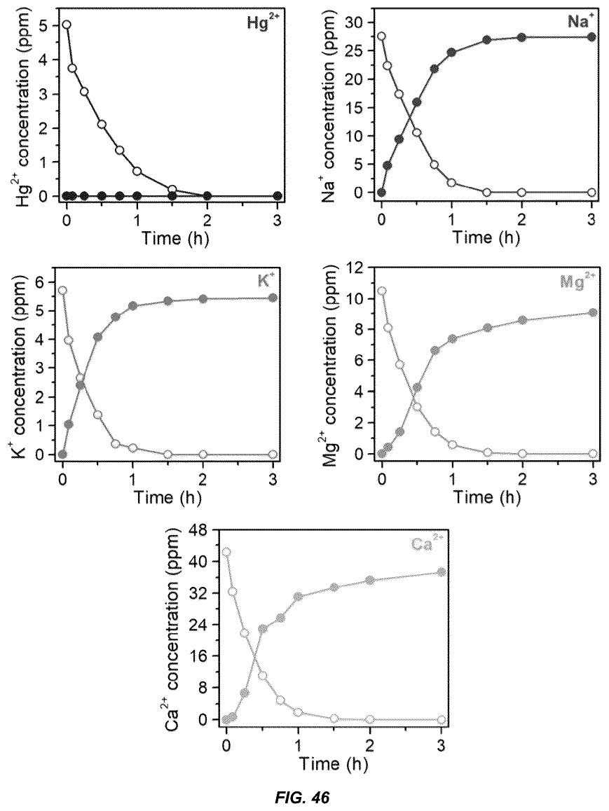

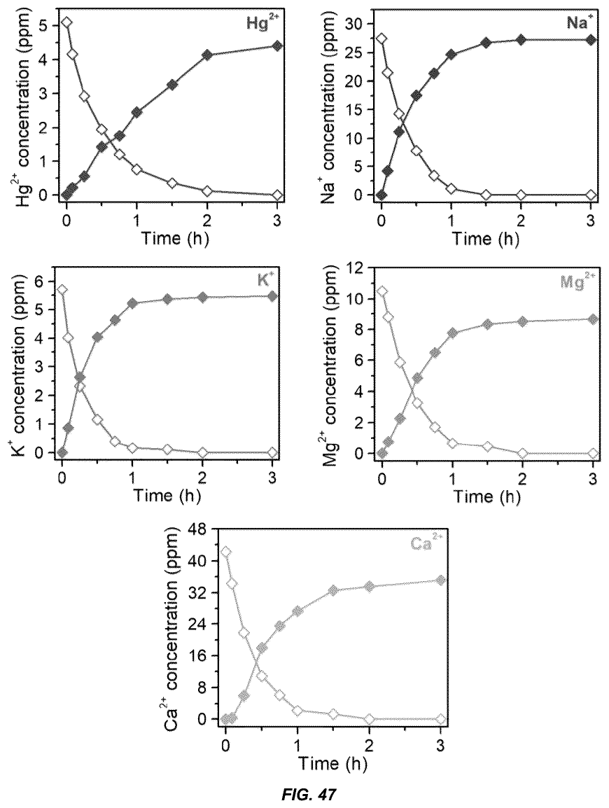

While stirring, 7.5 mL DI water containing 10 mM TraceMetal Grade HNO₃ was added to the cation receiving compartment, and 7.5 mL DI water containing 10 mM LiOH was added to the anion receiving compartment. These solutions were added to maintain electrical conductivity and neutralize hydroxide and protons formed at the cathode and anode, respectively. An aqueous solution (7.5 mL) containing Hg(NO₃)₂ (5 ppm Hg²⁺) spiked in synthetic groundwater was then added to the feed compartment. All the solutions were stirred for ˜10 s before aliquots were removed from each compartment; the concentrations in these samples corresponded to t=0. A constant voltage of 10 V was then immediately applied across the cell using a DC power supply (Nice-Power). Aliquots of the solutions (0.3 mL) in each compartment were collected and analyzed at fixed time intervals. The time-dependent cation concentration profiles in each compartment and ion-capture electrodialysis performance when using a 20 wt % PAF-1-SH in sPSF membrane are shown in FIGS. 44-46. Time-dependent cation concentration profiles in each compartment when using a neat sPSF membrane are shown in FIG. 47.

The percent of Hg²⁺ captured by the 20 wt % PAF-1-SH membranes from the feed solution was calculated using Eq. S9. The percent feed desalination (i.e., deionization, or the percentage of all ions removed from the feed) in the stack electrodialysis experiments was calculated using Eq. S11 to account for the removal of both cations and anions in the feed:

$$Stack desalination ( \% ) = 100 \% \times ( 1 - \frac{{δ}_{f , feed}}{{δ}_{0 , feed}} ) \qquad ( S11 )$$

where δ_{f, feed} and δ_{0, feed} are the conductivities of the feed solution at the final and initial (t=0) time points, respectively. These solution conductivities were measured using a conductivity meter (Thermo Scientific Orion Versa Star Pro pH/Conductivity Multiparameter Benchtop Meter). The measured conductivity of the initial feed solution was 532 pS cm⁻¹. The measured conductivity of the final feed solution was equal to the measured conductivity of the air-equilibrated DI water used (2.0 μS cm⁻¹). This conductivity was used as δ_{f, feed} when calculating the stack desalination percentage. Notably, the stack desalination rate calculated using Eq. S11 (>99.6%) approximately matched the desalination rate calculated using Eq. S9 (>99.7%), which was used in two-compartment electrodialysis experiments and was only based on measured cation concentrations.

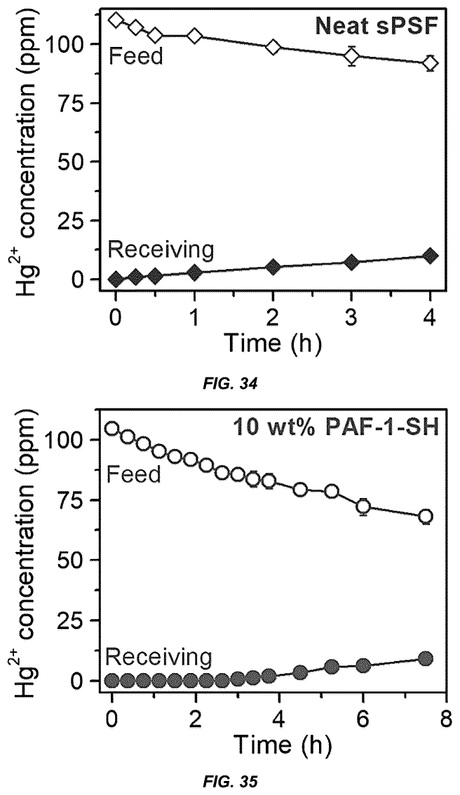

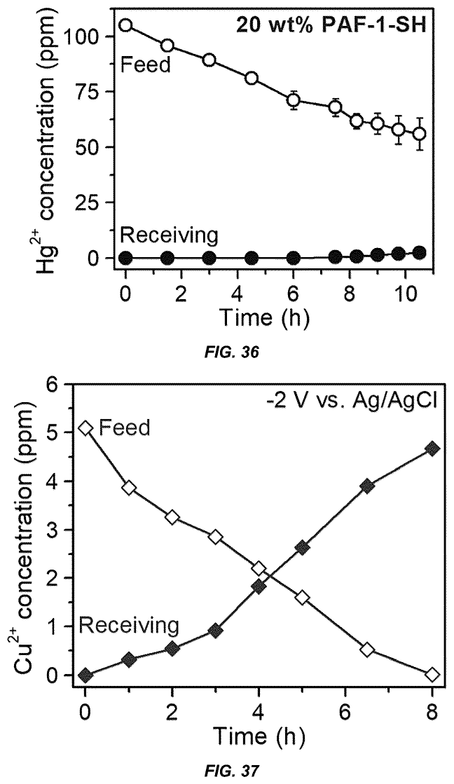

Ion-capture electrodialysis breakthrough. A hydrated membrane (neat sPSF, 10 wt % PAF-1-SH in sPSF, or 20 wt % PAF-1-SH in sPSF; 2.0 cm² active area) as the Na⁺ counterion form was clamped between two 45-mL half-cells. At room temperature, the solutions in both half-cells were constantly stirred at ˜1,100 rpm. A platinum counter electrode was placed in the feed half-cell, while a glassy carbon working electrode and a Ag/AgCl reference electrode (3 M NaCl internal electrolyte) was inserted in the receiving half-cell. While stirring, 45 mL DI water containing 1 mM HNO₃ (to maintain electrical conductivity and neutralize hydroxide formed at the cathode) were added to the receiving half-cell. An aqueous solution containing Hg(NO₃)₂ (45 mL, 100 ppm Hg²⁺ and a supporting electrolyte of NaNO₃ (0.1 M) were added to the feed half-cell. A voltage of −2 V vs. Ag/AgCl was applied using a BioLogic SP-200 potentiostat and EC-Lab software. Aliquots of the solutions (0.3 mL) in each half-cell were collected and analyzed at fixed time intervals. To collect any electrodeposited metals, the cathode was sonicated in concentrated HNO₃ (TraceMetal Grade) for ˜30 s each time an aliquot was collected from the receiving solution. Reported receiving half-cell concentrations represent the combined concentrations of this rinsing solution and the aliquot sample. No electrodeposited metals were observed on the anode. Hg²⁺ concentrations were measured via ICP-OES. Both half-cells were capped loosely with a rubber septum and vented to ambient air to remove H₂ and O₂ formed at the cathode and anode, respectively. No solution leakages in the cells were detected in any of the reported experiments for the entirety of the tests. The pH in each half-cell was measured to be between 6 and 8 throughout the entirety of the experiments. Reported values and error bars represent the mean and range, respectively, obtained from measurements on two different samples. The raw breakthrough data are presented in FIGS. 34 to 36.

Membrane breakthrough capacities (milligrams of Hg²⁺ captured per gram of dry PAF-1-SH in the membrane, FIG. 3D) were calculated using Eq. S5, based on the changes in Hg²⁺ concentration in the feed half-cell. Volume changes due to 0.3-mL aliquot sample removal were accounted for when calculating the amount of Hg²⁺ captured in the membranes. The theoretical breakthrough capacity (426 mg g⁻¹, FIG. 3D) was calculated as the percentage of accessible PAF-1-SH adsorption sites within the membrane matrix (93%, see Table F and FIG. 2C) multiplied by the Hg²⁺ capacity of PAF-1-SH powder at approximately equivalent testing conditions (458 mg g⁻¹, FIG. 23). Like conditions in the breakthrough tests, these adsorption testing conditions also consisted of an initial solution of 100 ppm Hg²⁺ in 0.1 M NaNO₃. The percentage of PAF ion-capture sites utilized in an IC-ED setup (96%, FIG. 3D) was then calculated as the experimentally measured Hg²⁺ breakthrough capacity (409 mg g⁻¹, FIG. 3D) divided by the theoretical Hg²⁺ breakthrough capacity.

Solute-capture diffusion dialysis. A similar setup as described for IC-ED experiments was used but without the insertion of electrodes or application of voltage across the half-cells.

B(OH)₃-capture dialysis of groundwater using boron-selective membranes. Membranes consisting of 20 wt % PAF-1-NMDG in sPSF were tested for B(OH)₃-capture dialysis. The hydrated membrane (2.0 cm² active area) in the Li⁺ counterion form was clamped between two 1.7-mL half-cells. The receiving half-cell was charged with 1.7 mL DI water. The feed half-cell was filled with a 1.7 mL aqueous solution of synthetic groundwater (containing B(OH)₃ (4.5 ppm boron, representing a typical concentration in seawater and within the typical concentration range in groundwater). At room temperature, the solutions in both half-cells were constantly stirred at ˜600 rpm. The half-cells were capped with multiple strips of Parafilm wrap to diminish evaporation. Aliquots of each half-cell solution (40 μL) were collected and analyzed at fixed time intervals. Boron concentrations were measured via ICP-OES. Samples were prepared for ICP-OES measurements by diluting in DI water, and calibration solutions were prepared using a boron ICP standard solution (VeriSpec, Ricca Chemical Company, Arlington, TX). Reported values and error bars represent the mean and range, respectively, obtained from measurements on two different samples. For comparison, the experiments were repeated using a neat sPSF membrane (FIG. 4C, inset). No solution leakages in the cells were detected in any of the reported experiments.

To test for possible boron leaching from the borosilicate glassware, we also carried out a control experiment using the same protocol as above, in the absence of a membrane and with the entire cell filled with 3 mL of the groundwater solution containing 4.5 ppm boron. No measurable changes in the solution boron concentration were observed over a one-week period.

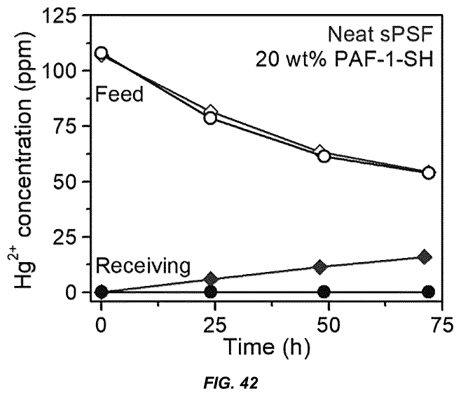

Hg²⁺-capture dialysis using mercury-selective membranes. 20 wt % PAF-1-SH in sPSF membranes were tested for Hg²⁺-capture diffusion dialysis. The hydrated membrane (2.0 cm² active area) in the Na⁺ counterion form was clamped between two 45-mL half-cells. The receiving half-cell was charged with 45 mL DI water. The feed half-cell was filled with a 45 mL aqueous solution containing Hg(NO₃)₂ (100 ppm Hg²⁺) and NaNO₃ (0.1 M) in DI water. At room temperature, the solutions in both half-cells were constantly stirred at ˜1,100 rpm. Aliquots (0.4 mL) of each half-cell solution were collected and analyzed at fixed time intervals. Electrode and sampling ports were otherwise closed off with screw caps. Hg²⁺ concentrations were measured via ICP-OES. For comparison, the experiments were repeated using a neat sPSF membrane. No solution leakages in the cells were detected in any of the reported experiments for the entirety of the tests. The Hg²⁺-capture diffusion dialysis results from both membrane types are shown in FIG. 42.

Water-stable PAF membranes with high charge density. Sulfonated polysulfone synthesized with a degree of sulfonation of 146% or higher swells dramatically upon immersion in water. Membranes fabricated using these hydrophilic, high-charge-density sPSF materials dissolve in water after casting and thus cannot be used in practical applications (figs. S7 and S21). However, crosslinking interfacial interactions between the PAFs and polymer backbone allow freestanding films to be fabricated after incorporating PAFs into these high-charge-density sPSF matrices.

The PAFs do not leach from the composite membranes upon submersion in water. In conjunction with the membrane dissolution tests, fabricated 20 wt % PAF-1-SH membranes were submerged in DI water for 24 h. No change in mass was measured following this submersion, indicating that no loss of PAF had occurred (FIG. 16). All membrane samples were stored in DI water when not in use. No apparent changes in the membrane appearance or water transparency were observed over the course of storage, which lasted over two years in some cases.

Electrodialysis time is an artifact of cell design. The relatively long durations of the IC-ED experiments (e.g., 24 h for Hg²⁺-capture electrodialysis of brackish water) are largely an artifact of the chosen experimental setup. For instance, the time required for the feed target ion concentration to completely diminish is expected to be much faster in a typical industrial electrodialysis setup. As a simplified analysis, this assertion is explained here by comparing the relative ratio of the feed solution volume to membrane active area in our setup to that in a typical industrial setup. This ratio was chosen as a comparison because these two parameters dictate the rate of feed ion concentration decrease, since a larger membrane active area increases the quantity of ions transported through the membrane, while a smaller feed solution volume increases the rate of concentration changes. A smaller ratio of the feed solution volume to membrane area is thus expected to lead to a shorter duration for an IC-ED process.

The custom-made electrodialysis setup has a feed volume of 7.5 cm³ and a membrane active area of 2.0 cm², yielding a feed volume to membrane area ratio of 3.75 cm. A typical industrial electrodialysis setup consists of a rectangular prismatic shape in which ion exchange membranes are placed parallel to each other in a stack and are separated by spacer gaskets with 0.3 to 2 mm thickness. Assuming a 2 mm spacer thickness and a 1 m² (i.e., 10,000 cm²) membrane area, a maximum feed solution volume of 2,000 cm³ is expected. Accordingly, a feed volume to membrane area ratio of 0.2 cm or lower is expected in a typical industrial electrodialysis setup, over an order of magnitude lower than the ratio of 3.75 cm in our setup. Therefore, assuming that ion transport driving forces are held constant (e.g., same applied potential and ion concentration gradients), the duration of an IC-ED experiment when using our setup is expected to be over an order of magnitude longer than when using typical industrial setups.

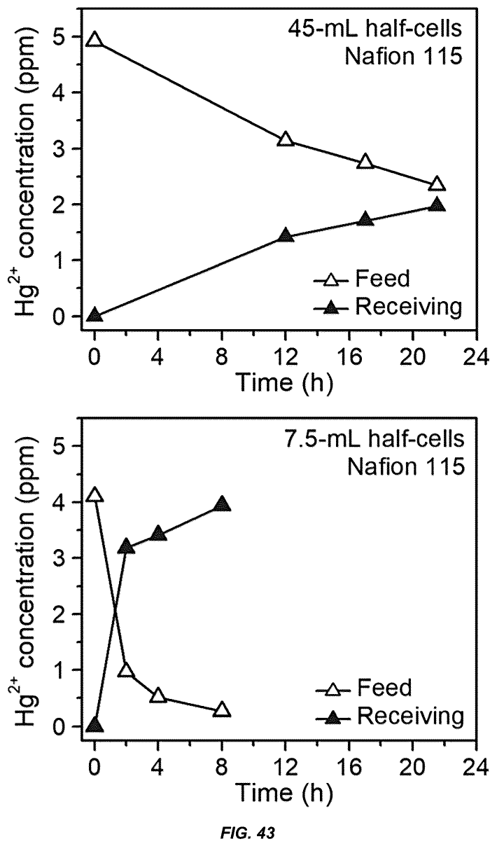

To validate the impact of the solution volume to membrane area ratio on electrodialysis experimental durations, the same electrodialysis experiment was conducted on two different sets of custom-made two-compartment cells, which each had a 2.0 cm² active area but different solution volumes. The first experiment featured 45 cm³ half-cells and a ratio of 22.5 cm for the feed volume to membrane area, while the second experiment featured 7.5 cm³ half-cells and a ratio (3.75 cm) six times smaller. Feed solutions of ˜4.5 ppm Hg²⁺ spiked in synthetic groundwater were used, and a 1 mM HNO₃ solution in DI water was added to the receiving half-cell. Nafion-115 membranes (Chemours, 127 μm thickness) converted to the Na⁺ counterion form were used to ensure consistency between the two experiments. A voltage of −2 V vs. Ag/AgCl was applied across the cells. As shown in FIG. 43, the feed Hg²⁺ concentration reduced by 2.5 ppm after 22 h when the larger half-cell volumes were used. However, the duration of the same experiment was over an order of magnitude shorter when the smaller half-cell volumes were used, as the feed Hg concentration reduced by 3.1 ppm after as little as 2 h when using the smaller half-cells (FIG. 43). This time reduction was even larger than expected based on the difference between the feed solution volume to membrane area ratios for each setup. These results corroborate the assertion that the relatively long durations of the IC-ED experiments in this report, compared to expected durations if a typical industrial setup was used, are largely an artifact of the cell design used.

To estimate the amount of water that can be treated in an ion-capture electrodialysis process before regeneration is required, the use of 20 wt % PAF-1-SH in sPSF membranes was used as representative adsorptive membranes in treating water samples containing Hg²⁺ as the target contaminant at concentrations of 5, 1, and 0.1 ppm. Volumes of water treated were calculated assuming that PAF-1-SH embedded in the membranes reaches full Hg²⁺ saturation as shown in FIG. 18, and that complete removal of Hg²⁺ from the feed water is achieved. Calculated volumes of water treated are provided in Table H, with values normalized by the amount of membrane used. The relative volumes of water treated compared to the desorption volumes required are additionally provided in Table H.

TABLE H
Calculated estimates of the amount of water that can be treated
by ion-capture electrodialysis before membrane regeneration
is required. Calculations were based on the use of a 20 wt % PAF-
1-SH in sPSF membrane to treat feed water contaminated with the
indicated concentrations of Hg²⁺.

| | | Water | Water |
|---|---|---|---|
| | | volume | volume |
| Initial | Water volume | treated per | treated per |
| feed Hg²⁺ | treated per | membrane | regeneration |
| concentration | membrane mass | volume | volume |
| (ppm) | (L kg⁻¹) | (L L⁻¹) ^{a} | (L L⁻¹) ^{b} |
| 5 | 34,500 | 32,100 | 690 |
| 1 | 172,500 | 160,500 | 3,450 |
| 0.1 | 1,725,000 | 1,605,000 | 34,500 |

^{a} Values were converted from water treated per membrane mass to volume treated per membrane volume by assuming the 20 wt % PAF-1-SH membrane has a density of 0.931 kg L⁻¹. This density was determined as the volume-averaged density of bulk PAF-1-SH and sPSF (0.420 kg L⁻¹ and 1.337 kg L⁻¹, respectively), using the 44.3 vol % PAF-1-SH value determined for a 20 wt % PAF-1-SH membrane (table S2).
^{b} Required regeneration volumes to enable 100% Hg²⁺ desorption were taken as 50 L per kg membrane, based on regeneration studies presented in FIG. 48. As this ratio is based on preliminary regeneration studies, in principle it may be further optimized to decrease the required regeneration volumes.

Estimates were also made of the amount of water that can potentially be treated in an ion-capture electrodialysis plant per regeneration cycle, based on a typical industrial electrodialysis design. Here, the same performance assumptions were made as described above and assume 20 wt % PAF-1-SH in sPSF membranes are implemented as the cation exchange membranes. While electrodialysis designs and sizes vary by plant, the following design parameters were assumed based on typical setups reported:
300 membrane stack pairs (i.e., 300 cation exchange membranes consisting of 20 wt % PAF-1-SH in sPSF)
1-m² active area per membrane
300-μm thickness for each membrane

Based on this design, a total 20 wt % PAF-1-SH membrane volume of 90 L, and thus a total PAF-1-SH mass of 16.8 kg, is expected for such a plant. The PAF-1-SH mass was determined by assuming that the 20 wt % PAF-1-SH membranes have a density of 0.931 kg L⁻¹. This density was determined as the volume-averaged density between bulk PAF-1-SH and sPSF (0.420 kg L⁻¹ and 1.337 kg L⁻¹, respectively), using the 44.3 vol % PAF-1-SH value determined for a 20 wt % PAF-1-SH membrane (table B). With the PAF-1-SH performance assumptions previously discussed, we estimate that the following volumes of water can be treated in an ion-capture electrodialysis plant before regeneration is required:
˜3,000,000 L of water treated for a feed source containing 5 ppm Hg²⁺
15,000,000 L of water treated for a feed source containing 1 ppm Hg²⁺
˜150,000,000 L of water treated for a feed source containing 0.1 ppm Hg²⁺

Ion-capture electrodialysis operating considerations. Operating conditions and setups for ion-capture electrodialysis processes are expected to mimic those used in traditional electrodialysis processes, with the key difference being that the membranes are replaced with selective adsorptive membranes that will need to be occasionally regenerated. The ion-capture electrodialysis process was designed to be compatible with traditional electrodialysis operating conditions to simplify its implementation into existing industrial setups. Similarly, solute-capture diffusion dialysis (and other multifunctional separation modalities based on the fundamentals uncovered in this report) are expected to operate under conditions similar to those used in traditional membrane processes (e.g., diffusion dialysis).

It will be understood that various modifications may be made without departing from the spirit and scope of this disclosure. Accordingly, other embodiments are within the scope of the following claims.

What is claimed is:

1. A composite membrane comprising a polymer/membrane matrix that contains or is embedded with particles comprising one or more types of porous aromatic frameworks (PAFs), wherein the one or more types of PAFs comprise functional groups that bind with a high specificity to a targeted ion, organic molecule, or contaminant.

2. The composite membrane of claim 1, wherein the polymer/membrane matrix comprises an ion exchange polymer/membrane matrix material.

3. The composite membrane of claim 2, wherein the ion exchange polymer/membrane matrix material is a cation exchange polymer/membrane matrix material.

4. The composite membrane of claim 2, wherein the ion exchange polymer/membrane matrix material is a anion exchange polymer/membrane matrix material.

5. The composite membrane of claim 2, wherein the ion exchange polymer/membrane matrix materials is made from dimethyl-2-hydroxy benzyl amine, phenol and formaldehyde; C₆H₄ (OH)₂ or 1,2,3-C₆H₃(OH)₃, NH₂C₆H₄COOH, and formaldehyde; benzidine-formaldehyde and acrylonitrile-vinyl chloride copolymer; phenolsulfonic acid and formaldehyde; m-phenylene diamine or aliphatic diamine compounds and formaldehyde; tetrafluoroethylene and vinyl-ether; sulfonation and amination of styrene and divinylbenzene polymers; and sulfonated polysulfone.

6. The composite membrane of claim 5, wherein the ion exchange polymer/membrane matrix material is made from sulfonated polysulfone.

7. The composite membrane of claim 1, wherein the particles are from 50 nm to 300 nm in diameter.

8. The composite membrane of claim 1, wherein the particles are evenly dispersed in the polymer/membrane matrix material.

9. The composite membrane of claim 1, wherein the composite membrane contains from 5 wt % to 40 wt % of the one or more types of PAFs.

10. The composite membrane of claim 9, wherein the composite membrane contains from 10 wt % to 25 wt % of the one or more types of PAFs.

11. The composite membrane of claim 1, wherein the one or more types of PAFs comprise a series of nodes linked together by linking ligands,
wherein the series of nodes have a formula selected from Formula I or Formula II: 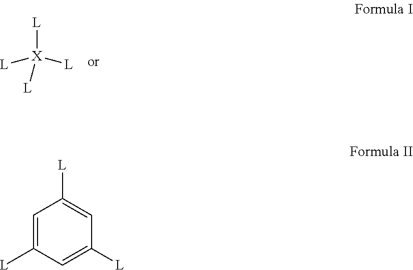
wherein, X is selected from C, B⁻ and P⁺; and L is a linking ligand; and
wherein the linking ligand has a structure of Formula III: 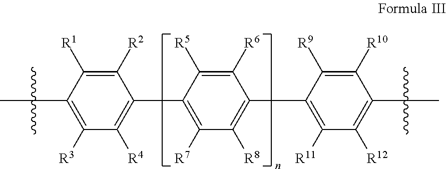
wherein, R¹-R¹² are independently selected from H, an optionally substituted (C₁-C₆)alkyl, an optionally substituted (C₁-C₆)alkenyl, an optionally substituted (C1-C5)-O-(C₁-C₆)alkyl, halo, —OH, —CH₂R¹³, —CO₂H, —COR¹⁴, —CO₂R¹⁴, —SH, —SMe, —SO₂H, —SO₃H, —NR¹⁵R¹⁶, —N⁺(H)₃, —N⁺(CH₃)₃, cyano, amide, azide, —PO₃H, —B(ORR¹⁴)₂, 2-(methylthio)ethan-1-ol, N-methyl-D-glucamine, and heterocycle;
R¹³ is selected from H, —OH, halo, —NH₂, —NR¹⁵R¹⁶, —N═C (CH₃)₂, phthalimide, —C(NH₂)═N—OH, —SH, —SMe, —SO₂H, —SO₃H, —N⁺(H)₃, —N⁺(CH₃)₃, —PO₃H, —O-(C₁-C₆)alkyl, cyano, amide, azide, —B(OR¹⁴)₂, - and heterocycle;
R¹⁴ to R¹⁶ are independently selected from H or an optionally substituted (C₁-C₆)alkyl; and
n is an integer selected from 0, 1, 2, 3, 4, or 5.

12. The composite membrane of claim 1, wherein the one or more types of PAFs are selected from PAF-1, PAF-1-CH₃, PAF-1-CH₂OH, PAF-1-CH₂-phthalimide, PAF-1-CH₂N═CMe₂, PAF-1-CH₂Cl, PAF-1-SH, PAF-1-ET (wherein ET is 2-(methylthio)ethan-1-ol), PAF-1-NMDG (wherein NMDG is N-methyl-D-glucamine), PAF-1-SMe, PAF-1-CH₂NH₂, and PAF-1-CH₂AO (wherein AO is an amidoxime group).

13. The composite membrane of claim 12, wherein the one or more types of PAFs are selected from PAF-1-SH, PAF-1-ET, PAF-1-NMDG, PAF-1-SMe, PAF-1-CH₂NH₂, and PAF-1-CH₂AO.

14. The composite membrane of claim 1, wherein the one or more types of PAFs bind with a high specificity to a targeted ion selected from Hg²⁺, Nd³⁺, Cu²⁺, UO₂²⁺, B(OH)₃, Fe³⁺, and AuCl₄⁻.

15. A method for removing a targeted ion, organic molecule, or contaminant from a fluid feedstock:
contacting a fluid feedstock with a composite membrane of claim 1, wherein the fluid feedstock comprises the targeted ion, organic molecule, or contaminant,
wherein the targeted ion, organic molecule, or contaminant is absorbed or captured by the PAFs present in the composite membrane.

16. The method of claim 15, wherein the fluid feedstock is used in an application or process selected from: electrodialysis, membrane capacitive deionization, electrofiltration, fuel cells, fuel gas streams, gas purification, and CO₂ capture.

17. The method of claim 15, wherein non-targeted ions, organic molecules, or contaminants flow through the membrane and are not absorbed or captured by the PAFs present in the composite membrane.

18. The method of claim 15, wherein the fluid feedstock comprises a targeted ion selected from Hg²⁺, Nd³⁺, Cu²⁺, Pb²⁺, UO₂²⁺, B(OH)₃, Fe³⁺, and AuCl₄⁻.

19. The method of claim 15, wherein the fluid feedstock comprises a targeted contaminant selected from carbon dioxide, hydrogen sulfide, nitrogen, water, and sulfur oxide.
